SEQUENCE LISTING (4 sequences)

SEQ ID NO: 1 - protein, 79 aa, Artificial sequence
  Synthetic
MGSSHHHHHHSSGLVPRGSHMAEAKYAKMRNAYWEIALLPNLTNQQKRAFIRKLYDDPSQSSELLSEAKKLNDSQAPKC

SEQ ID NO: 2 - protein, 53 aa, Homo sapiens
NSDSECPLSHDGYCLHDGVCMYIEALDKYACNCVVGYIGERCQYRDLKWWELR

SEQ ID NO: 3 - protein, 7 aa, Artificial Sequence
  Synthetic
  MOD_RES (1)..(1): X is 8-aminooctanoic acid.
XFGWWGC

SEQ ID NO: 4 - protein, 6 aa, Artificial Sequence
  Synthetic
  MOD_RES (1)..(1): X is 8-aminooctanoic acid
  MOD_RES (4)..(4): X is diaminopropionic acid
XFFXDC

(19) United States
(12) Patent Application Publication
(10) Pub. No.: US 2022/0175814 A1
(43) Pub. Date: Jun. 9, 2022

(54) IMMUNOTHERAPY FOR THE TREATMENT OF CANCER

(71) Applicant: TARGIMMUNE THERAPEUTICS AG, Basel (CH)

(72) Inventors: Derrick BROKA, Luzern (CH); David COLECCHIA, Basel (CH); Lucia D'AMICO, Basel (CH); Eric KITAS, Aesch (CH); Alexander LEVITZKI, Jerusalem (IL); Esteban POMBO-VILLAR, Binningen (CH); Alexei SHIR, Jerusalem (IL); Maya ZIGLER, Basel (CH); Davor BAJIC, Basel (CH); Anita JARZEBINSKA, Basel (CH)

(21) Appl. No.: 17/594,054

(22) PCT Filed: Apr. 3, 2020
(86) PCT No.: PCT/EP2020/059689

§ 371 Date: Sep. 30, 2021

(30) Foreign Application Priority Data
Apr. 3, 2019 (EP) 19167141.1

(51) Int. Cl. A61K 31/713; A61K 47/60; A61K 47/64; A61P 35/00; C07K 16/28; C07K 16/32; C07K 17/06

(52) U.S. Cl. CPC A61K 31/713; A61K 47/60; A61K 47/642; A61K 47/646; C07K 17/06; C07K 16/2818; C07K 16/2878; C07K 16/32; A61P 35/00

(57) ABSTRACT

The present invention relates to a kit-of-parts comprising and a composition comprising a polyplex comprising a double stranded RNA (dsRNA) and a polymeric conjugate comprising a polyethyleneimine (PEI), one or more polyethylene glycol (PEG) moieties and one or more targeting moieties, and wherein each of said one or more targeting moieties is capable of binding to a cancer antigen; and at least one antibody, wherein said at least one antibody is capable of modulating an immune checkpoint protein. Further the invention relates to this composition or kit-of-parts for use in the treatment of cancer.

The present invention relates to the field of immunotherapeutic cancer treatment. Especially, the present invention relates to a kit-of-parts and a composition comprising a polyplex comprising a double stranded RNA (dsRNA) and a polymeric conjugate comprising a polyethyleneimine (PEI), one or more polyethylene glycol (PEG) moieties and one or more targeting moieties, and wherein each of said one or more targeting moieties is capable of binding to a cancer antigen; and at least one antibody, wherein said at least one antibody is capable of modulating an immune checkpoint protein. Further the invention relates to this composition or kit-of-parts for use in the treatment of cancer.

### RELATED ART

Antibodies that target tumor-associated antigens have become an important treatment modality for malignancies. Several monoclonal antibodies (mAbs) have already proved to be relatively well-tolerated and effective for the treatment of many different malignant diseases. Although these antibodies are commonly used in the clinic, their efficacy is often modest. mAbs must overcome substantial obstacles to reach antigens presented on target cells to be of therapeutic value (Christiansen et al., Mol Cancer Ther, 2004, 3(11), 1493-1501). Moreover, efficiency of antibodies that target tumor-associated antigens is lowered by insufficient activation of the anti-tumor response of the immune system and by inhibition of the immune reaction induced by the tumor itself.

Checkpoint blockade antibodies targeting cytotoxic T-lymphocyte antigen 4 (CTLA-4) and programed cell-death protein 1 (PD-1) have demonstrated acceptable toxicity, promising clinical responses, durable disease control, and improved survival in some patients with advanced melanoma, non-small cell lung cancer (NSCLC), and other tumor types. Engagement of PD-1 by its ligands, either PD-L1 or PD-L2, induces a negative control signal resulting in the inhibition of T cell proliferation, cytokine production, and cytotoxic activity. (Ma et al., Current status and perspectives in translational biomarker research for PD-1/PD-L1 immune checkpoint blockade therapy, Journal of Hematology & Oncology (2016) 9:47).

Cytokines related to tumor necrosis factor (TNF) provide a communication network essential for coordinating multiple cell types into an effective host defense system against pathogens and malignant cells. The tumor necrosis factor superfamily of ligands (TNFSF) and receptors (TNFRSF) provide key communication signals between various cell types during development. FTNF receptors (TNFRs) share a conserved ectodomain defined by a cysteine-rich signature. The TNFRs with a co-stimulatory reputation are encoded by genes residing within an immune-response locus in chromosomal region 1p36 and include GITR (glucocorticoid-induced tumor necrosis factor), OX40, 4-1BB, and CD30 (Ward-Kavanagh, et al., The TNF Receptor Superfamily in Co-stimulating and Co-inhibitory Responses, Immunity 44, May 17, 2016).

A different approach for the treatment of malignancies is a vaccine-based therapy. The molecular definition of tumor-associated antigens introduced the possibility of specific vaccines aiming to target the tumor cells. Recombinant vaccines, which are based on peptides or proteins from defined tumor-associated antigens (TAAs) are usually administered together with an adjuvant or an immune modulator. Although these vaccines were able to induce antigen-specific T cell responses, clinical outcomes have been disappointing (Guo et al., Adv Cancer Res, 2013, 119: 421-475).

A further approach in cancer immunotherapy refers to the combination of vaccines with antibodies that activate antitumor immunity by blocking or inhibiting immune checkpoints. Immune checkpoints refer to a plethora of inhibitory pathways hardwired into the immune system that are important for maintaining self-tolerance and modulating the duration and amplitude of physiological immune responses. Tumors co-opt certain immune-checkpoint pathways as a major mechanism of immune resistance. Because many of the immune checkpoints are initiated by ligand-receptor interactions, they can be readily blocked or inhibited by antibodies or modulated by recombinant forms of ligands or receptors. Cytotoxic T-lymphocyte-associated antigen 4 (CTLA-4) antibodies were the first of this class of immunotherapeutics to achieve US Food and Drug Administration (FDA) approval. However, the development of combinatorial approaches of vaccines with antibodies is still a challenge. Combinatorial strategies have to be intelligently designed and guided by mechanistic considerations and preclinical models (Pardoll, Nat Rev Cancer 2012, 12(4), 252-264).

Several novel combinations for immunotherapy in oncology have been suggested (Morrisey et al. Clin. Transl. Sci 2016, 9, 89-104) including non-antigen specific immunotherapy with naked polyIC and blocking antibodies targeting the programmed cell death-1 (PD-1) pathway which was able of inhibiting tumor in cancer mouse models of B16 melanoma, Lewis lung carcinoma, and MC38 colon carcinoma (Nagato et al., OncoImmunology 2014, 3: e28440).

Despite first encouraging results, the development of immunotherapeutics for cancer continues to be a major challenge for tumor immunologists. Thus, there is a high need for an effective and well-tolerated immunotherapy for the treatment of cancer.

### SUMMARY OF THE INVENTION

The present invention provides a novel combinatorial immunotherapeutic approach for the treatment of cancer.

In a first aspect, the present invention provides for a kit-of-parts comprising

a. a polyplex comprising a double stranded RNA (dsRNA) and a polymeric conjugate, wherein said polymeric conjugate comprises a polyethyleneimine (PEI), one or more polyethylene glycol (PEG) moieties and one or more targeting moieties, wherein said PEI is covalently bound to one or more PEG moieties, and each of said one or more PEG moieties is linked to one of said one or more targeting moieties, and wherein each of said one or more targeting moieties is capable of binding to a cancer antigen; and

b. at least one antibody, wherein said at least one antibody is capable of modulating an immune checkpoint protein.

In a further aspect, the present invention provides for a composition comprising

a. a polyplex comprising a double stranded RNA (dsRNA) and a polymeric conjugate, wherein said polymeric conjugate comprises a polyethyleneimine (PEI), one or more polyethylene glycol (PEG) moieties and one or more targeting moieties, wherein said PEI is covalently bound to one or more PEG moieties, and each of said one or more PEG moieties is linked to one of said one or more targeting moieties, and wherein each of said one or more targeting moieties is capable of binding to a cancer antigen; and

b. at least one antibody, wherein said at least one antibody is capable of modulating an immune checkpoint protein.

In a further aspect, the present invention provides for the composition or the kit-of-parts according to the invention for use in the treatment of cancer in a mammal.

The inventors surprisingly found that combination treatment with the polyplex comprising double stranded RNA (dsRNA) and the polymeric conjugate and one or more anti-checkpoint antibodies according to the invention enforces activity of the immune system and leads to potent antitumor activity. Tumor growth was inhibited more potently by the combination of the polyplex comprising dsRNA and the polymeric conjugate plus one or more anti-checkpoint antibodies as compared to the polyplex alone. In some individuals the combination of the polyplex and the anti-checkpoint antibody completely eradicated tumors and generated a sustained tumor defense and memory against the cancer cells.

Interferon secretion was increased by the combination of the polyplex of the invention plus an anti-checkpoint antibody as compared to the polyplex alone. Exposing immune cells, preferably peripheral blood mononuclear cells (PBMCs), to medium from EGFR overexpressing cells that had been treated with the polyplex of the invention, or culturing said immune cells in the presence of cells treated with the polyplex of the invention induced the PBMCs to secrete interferon. Adding an anti-checkpoint antibody thereto led to a further increase in interferon secretion (cf. FIGS. 3 and 4).

In immunocompetent mice bearing HER2 overexpressing tumors, mice showed complete regression of tumors after combination treatment with an anti-checkpoint antibody, preferably a monoclonal anti-PD-1 antibody, and the polyplex of the invention. Tumor re-challenge in cured mice did not induce tumor growth indicating that an immune response against the tumor has been generated.

Treatment of tumors with immunomodulatory antibodies alone is found to be not effective or showed only limited efficacy. Anti-PD-1 antibodies alone did not reduce tumor size in RENCA tumors (FIG. 5). However, the combination of the targeted and specifically delivered dsRNA and the immunomodulatory antibodies showed an increased anti-tumor effect. The treatment of RENCA HER2 tumors with a HER2 targeted polyplex of the invention, namely a triconjugate comprising PEI-PEG-HER2 affibody (PPHA) forming a complex with polyIC (pIC/PPHA)+anti-PD-1 surprisingly generated regression of tumor growth (FIG. 6). Complete regression of tumors was observed in combination arm with polyIC/PPHA+anti-PD-1. Cured mice showed complete protection from tumor re-challenge indicating that an immune response against the tumor has been generated.

Thus, the combination of the targeted polyplex and anti-checkpoint antibodies in accordance with the present invention is capable of broadening the efficacy of the antibodies to patients that are currently not showing any response. Utilizing targeted delivery of dsRNA, preferably polyIC in combination with anti-checkpoint antibodies shows significant efficacy due to the capabilities of the inventive compositions and kits-of-parts to reinstate the immune system against the tumor.

### DESCRIPTION OF FIGURES

FIG. 1: IP-10 secretion from cancer cells following treatment with PEI-PEG-EGF/polyIC. A431, U87 and MCF7 cells (40,000 cells per well) were treated for 5 hours with PEI-PEG-EGF/polyIC at various concentrations (0.125, 0.25, 0.5, 1 μg/ml). Human IP-10 (CXCL10) secretion was higher in A431 EGFR expressing cells compared to U87 and MCF7 cells expressing very low amount of EGFR.

FIG. 2: PD-L1 (interchangeable used herein and equivalent to “PDL1”) expression on A431 cells following treatment with PEI-PEG-EGF/polyIC. A431 cells were treated for 5 hours with PEI-PEG-EGF/polyIC at a concentration of 0.125 μg/ml. PD-L1 expression significantly increased (MFI=751, (C)) after PEI-PEG-EGF/polyIC treatment compared to untreated control cells (MFI=431, (B)). Isotype control used as negative control (MFI=13, (A)).

FIG. 3: Combination therapy of PEI-PEG-EGF/polyIC with Nivolumab significantly increased IFN-y production by PBMCs. Combining PEI-PEG-EGF/polyIC with Nivolumab significantly increased IFN-y production by PBMCs challenged with diluted media containing PEI-PEG-EGF/polyIC alone (0.125 μg/ml) or in combination with Nivolumab (20 μg/ml) for 48 hours.

FIG. 4: Combining PEI-PEG-EGF/polyIC with 4-1BB antibodies significantly increased IFN-y production by PBMCs. PBMCs were stimulated with CD3 (0.5 μg/ml) and co-cultured with A431 cells treated with PEI-PEG-EGF/polyIC alone (0.5 μg/ml) or in combination with antibodies against 4-1BB (10 μg/ml) for 16 hours.

FIG. 5: Antitumor activity of RMP1-14 in the treatment of s.c. RENCA Murine Kidney Cancer Model: No anti-tumor activity of anti-PD-1 (interchangeable used herein and equivalent to anti-PD1) antibodies (RMP1-14) in the treatment of subcutaneous tumors in the RENCA murine kidney cancer xenograft model (Syngeneic Models for Developing Cancer Therapeutics Targeting Immune System, Lan Zhang et al., EORTC-NCI-AACR International Conference on Molecular Targets and Cancer Therapeutics, Nov. 18-21, 2014, Barcelona, Spain, P013).

FIG. 6: Effect of a polyplex of polyIC and the chemical vector polyethylenimine-polyethyleneglycol (PP) conjugated to a HER2 affibody (HA), shortly called herein polyIC/PPHA, plus anti-PD-1 (polyIC/PPHA+PD-1) on s.c. RENCA HER2 tumor growth in immunocompetent mice (s.c. RENCA HER2 xenograft model). Mice bearing s.c. RENCA HER2 tumors were randomized into 4 groups, 7-8 animals/group with average tumor volume of 235 mm³. Mice were treated i.v. with polyIC/PPHA, 6.25 mg/mouse, N/P 8, every 24 h. Anti-PD-1 was injected i.p., 200 mg/mouse at indicated time points. Although there seems to be no significant difference between polyIC/PPHA alone and combination therapy of polyIC/PPHA+anti-PD-1, two mice in polyIC/PPHA+anti-PD-1 group are still tumor free 50 days after initiation of treatment.

FIG. 7: Individual tumor volume at day 21 in immunocompetent mice (s.c. RENCA HER2 xenograft model). Complete regression of tumor growth was observed in 2 mice out of 8 mice in polyIC/PPHA+anti-PD-1 treated animals. These mice remained tumor free after re-challenge.

FIG. 8: Increased expression of PD-L1 in RENCA HER2 cells following treatment with polyIC/PPHA. Calculated raw values of medians using X-axis channel(s): PE-A

FIG. 9: Effect of PEI-PEG-EGF/polyIC+antiPD-1 on Renca EGFR lung metastases in immunocompetent mice.

FIG. 10: Combination of PEI-PEG-EGF/polyIC polyplex and Nivolumab increases PBMC activation as demonstrated by IFN-γ (interchangeable used herein and equivalent to IFNγ) ELISA. A431 or medium alone were treated with PEI-PEG-EGF/polyIC polyplex at the indicated concentrations for 5 hours. PBMCs were stimulated with anti-CD3 or not stimulated, and treated with or without Nivolumab. Then, supernatant from PEI-PEG-EGF/polyIC polyplex-treated or untreated (UT) A431 cancer cells or PEI-PEG-EGF/polyIC polyplex in medium or untreated medium was transferred to the PBMCs. After overnight incubation, IFN-γ ELISA was performed to quantify PBMC activation.

FIG. 11: PEI-PEG-EGF/polyIC polyplex induces cytokine secretion. Three cell lines, high EGFR (Epidermal Growth Factor Receptor)-expressing cells (MDA-MB-468 and A431) and low EGFR-expressing cells (MCF7) were treated with PEI-PEG-EGF/polyIC polyplex, PEI-PEG-EGF triconjugate or pIC at the indicated concentrations for 5 h. Medium was then collected and analyzed utilizing ELISA assay for (A) IP10, (B) GROα and (C) CCL5 (RANTES).

FIG. 12: PEI-PEG-EGF/polyIC polyplex induces PBMC activation as measured by IFN-γ and TNFa ELISAs. Three cell lines MDA-MB-468 (high EGFR), A431 (high EGFR), and MCF7 (low EGFR), or medium alone were treated with PEI-PEG-EGF/polyIC, pIC and PEI-PEG-EGF/pLGA polyplexes, with the indicated concentrations of pIC or pLGA (poly-L-Glutamic-Acid) within the polyplexes for 5 hours. Supernatant and medium were collected and transferred to PBMCs for 16 hours. Supernatant from treated cancer cells without PBMCs was used as a control and incubated for 16 hours as well. IFN-γ and TNFa were quantified using ELISAs following treatment of MDA-MB-468 (A, D); A431 (B, E); and MCF7 cells (C, F) respectively.

FIG. 13: PEI-PEG-EGF/polyIC polyplex treatment induces cancer cell death in EGFR-overexpressing cells with high efficacy and selectivity as compared to control treatments. Cancer cells with differential EGFR expression levels were treated with the following reagents: PEI-PEG-EGF/polyIC, pIC alone (naked pIC), jetPEI-pIC, RNAiMax-pIC, and PEI-PEG-EGF/pLGA polyplexes, for 72 h. The concentrations shown reflect the concentrations of pIC or pLGA. Cell survival was analyzed using Celtiter-Glo. For each compound, percent survival was normalized to that of untreated cells. (A, B) High EGFR-expressing cell lines, BT20, MDA-MB-468, A431 and HCC70, (C) medium EGFR-expressing cell line, U87MG, (D) low EGFR-expressing cell lines U138 and MCF7 and (E) non-cancer cell lines WI-38 and MCF10A.

### DETAILED DESCRIPTION OF THE INVENTION

Unless defined otherwise, all technical and scientific terms used herein have the same meanings as commonly understood by one of ordinary skill in the art to which this invention belongs.

Throughout this specification and the claims which follow, unless the context requires otherwise, the word “comprise” or the word “include”, and variations such as “comprises/includes” and “comprising/including”, are to be understood to imply the inclusion of an element, stated integer, step or a group thereof but not the exclusion of any other element, stated integer, step or a group thereof.

As used in this specification and the appended claims, the singular forms “a”, “an”, and “the” include plural referents, unless the content clearly dictates otherwise.

The term “about” or “approximately” when used in connection with a numerical value is meant to encompass numerical values within a range having a lower limit that is 0-10% smaller than the indicated numerical value and having an upper limit that is 0-10% larger than the indicated numerical value. The term “about” or “approximately” means preferably ±10%, more preferably ±5%, again more preferably +3% or most preferably +0% (referring to the given numeric value, respectively). In each of the invention embodiments, “about” can be deleted. All ranges of values disclosed herein, should refer and include to any and all values falling within said range including the values defining the range.

In one aspect, the invention refers to a composition comprising

a. a polyplex comprising a double stranded RNA (dsRNA) and a polymeric conjugate,

wherein said polymeric conjugate comprises a polyethyleneimine (PEI), one or more polyethylene glycol (PEG) moieties and one or more targeting moieties, wherein said PEI is covalently bound to one or more PEG moieties, and each of said one or more PEG moieties is linked to one of said one or more targeting moieties, and wherein each of said one or more targeting moieties is capable of binding to a cancer antigen; and

b. at least one antibody, wherein said at least one antibody is capable of modulating an immune checkpoint protein.

In a further aspect, the invention refers to a kit-of-parts comprising

a. a composition, wherein said composition comprises a polyplex comprising a double stranded RNA (dsRNA) and a polymeric conjugate, wherein said polymeric conjugate comprises a polyethyleneimine (PEI), one or more polyethylene glycol (PEG) moieties and one or more targeting moieties, wherein said PEI is covalently bound to one or more PEG moieties, and each of said one or more PEG moieties is linked to one of said one or more targeting moieties, and wherein each of said one or more targeting moieties is capable of binding to a cancer antigen; and

b. at least one antibody, wherein said antibody is capable of modulating an immune checkpoint protein.

In a preferred embodiment, said composition of the invention comprises at least one pharmaceutically acceptable diluent, excipient or carrier. In a particularly preferred embodiment, the composition according to the invention is a fixed-dose composition that comprises the polyplex and one or more immunomodulatory antibodies in a single dosage form. In another preferred embodiment, said pharmaceutical composition includes one or more adjuvants.

The term “kit-of-parts” as used herein preferably refers to a combination of at least two separate parts, namely said polyplex and said one or more immunomodulatory antibodies. In a preferred embodiment, said composition is a pharmaceutical composition. The arrangement and construction of such kit-of-parts is conventionally known to one skilled in the art. In a particularly preferred embodiment, said kit-of-parts of the invention or said parts of the kit-of-parts of the invention, i.e. the polyplex and/or the one or more immunomodulatory antibodies comprise independently of each other at least one pharmaceutically acceptable diluent, excipient or carrier. In another preferred embodiment, said kit-of-parts of the invention or said parts of the kit-of-parts of the invention, i.e. the polyplex and/or the one or more immunomodulatory antibodies comprise independently of each other one or more adjuvants.

In certain embodiments, the composition and kit-of-parts according to the invention is formulated for administration by any known method. The composition and kit-of-parts according to the invention, i.e. the polyplex and/or the one or more immunomodulatory antibodies, and the pharmaceutical composition may be formulated for any suitable route of administration including but not limited to intravenous, intra-brain (intracerebral), oral, intramuscular, subcutaneous, transdermal, intradermal, transmucosal, intranasal, sublingual, intraperitoneal or intraocular administration.

In another more preferred embodiment, said composition or kit-of-parts according to the invention is formulated for systemic administration. Again more preferably, the composition and kit-of-parts according to the invention, i.e. the polyplex and/or the one or more immunomodulatory antibodies, are formulated for intravenous, intraperitoneal, or subcutaneous administration. More preferably, the composition and kit-of-parts according to the invention, i.e. the polyplex and/or the one or more immunomodulatory antibodies, are formulated as one or more dosage forms suitable for injection, preferably as solution, emulsion or suspension suitable for injection.

In one embodiment, the composition and kit-of-parts according to the invention comprising the polyplex of the invention and one or more immunomodulatory antibodies, wherein said polyplex and said one or more immunomodulatory antibodies are present in the composition and kit-of-parts in a therapeutically effective amount.

Said kit-of-parts of the invention may include containers that contain the polyplex and/or the one or more antibodies and/or an apparatus for administering the parts of the kit, i.e. the polyplex and/or the one or more antibodies. In a preferred embodiment, said kit-of-parts of the invention comprises at least one container comprising an effective dose of said polyplex and at least one container comprising an effective dose of said one or more antibodies, and optionally an instruction leaflet.

The term “immune checkpoint protein” or “immune checkpoint” is known and described in the art (see for instance Pardoll, 2012, Nature Rev Cancer 12: 252-264; Darvin et al., 2018, Experimental & Molecular Medicine 50:165). The term “immune checkpoint protein” as used herein refers to receptors of T cells, B cells and natural killer cells (NKs) as well as their soluble or bound ligands and counter-receptors that can stimulate or inhibit activity of the immune system. In a preferred embodiment, said immune checkpoint protein refers to receptors of T cells and natural killer cells as well as their soluble or bound ligands and counter-receptors that can co-stimulate or co-inhibit activity of the immune system. Preferably, said activity of the immune system is detected by measuring a T cell response, as shown herein (e.g. Example 2).

Immune checkpoints proteins are important immune regulators in maintaining immune homeostasis and preventing autoimmunity. These consist of both stimulatory and inhibitory receptors and ligands that are important for maintaining self-tolerance and regulating the type, magnitude, and duration of the immune response. Immune homeostasis is regulated by a careful balance of activating and inhibitory immune checkpoint proteins. Under normal circumstances, immune checkpoints allow the immune system to respond against infection and malignancy while protecting tissues from any harm that may derive from this action.

Tumor cells have developed several strategies to exploit these checkpoints and circumvent the host immune defenses. Expression of immune checkpoint proteins can be dysregulated by tumors as an important immune resistance mechanism. Thus, inhibition or activation of checkpoint receptors and ligands have emerged as potential strategies to restrict tumor infiltrating lymphocytes (TIL) inhibiting signals from the tumor and circulating monocytes, block negative signals and cytokines that inhibit T cell activity, and stimulate systemic immunity.

Among the most promising approaches to activating therapeutic anti-tumor immunity is the blockade of inhibiting immune checkpoint proteins or the activation of stimulatory immune checkpoint proteins. Immune checkpoints refer to inhibitory and activating proteins of the immune system that are crucial for maintaining self-tolerance and modulating the duration and amplitude of physiological immune responses in order to minimize collateral tissue damage. Tumors co-opt certain immune-checkpoint pathways as a major mechanism of immune resistance, particularly against T cells that are specific for tumor antigens. T cells have been the major focus of efforts to therapeutically manipulate endogenous anti-tumor immunity owing to: their capacity for the selective recognition of peptides derived from proteins in all cellular compartments; their capacity to directly recognize and kill antigen-expressing cells (by CD8+ effector T cells; CTLs); and their ability to orchestrate diverse immune responses (by CD4+ helper T cells), which integrates adaptive and innate effector mechanisms. Thus, agonists of co-stimulatory receptors or antagonists of inhibitory signals, both of which result in the amplification of antigen-specific T cell responses, are the agents in current clinical testing. Because many of the immune checkpoints are initiated by ligand-receptor interactions, they can be readily modulated by antibodies.

Preferably, the immune checkpoint protein is a human immune checkpoint protein.

Within the present invention, an antibody capable of modulating an immune checkpoint protein is any compound that modulates the function of an immune checkpoint protein and thus promotes activity of the immune system. Promotion of immune system activity includes generation of enhanced immune responses to the antigen and/or reduction in immunosuppressive immune responses against the antigen. Preferably, promotion of immune system activity results in immune-mediated elimination of tumor cells.

The term “modulating” (or “modulator”) includes activation which relates to functional stimulation or enhancement of a co-stimulatory immune checkpoint protein as well as inhibition of a co-inhibitory immune checkpoint protein which relates to reduction in activity and full blockade of a co-inhibitory immune checkpoint protein. The designation “modulating an immune checkpoint protein” includes stimulation of T cells including T helper cells, CTLs natural killer T cells, and natural killer cells (NK). Stimulation (or activation) induced by modulation of an immune checkpoint protein is preferably detected via measuring increased levels of cytokines such as interferon, especially IFN-γ, produced or released, in particular, by T and NK cells as compared to controls without administration of immune checkpoint protein modulating antibodies, as shown herein (e.g. Example 2).

Examples of immune checkpoint proteins include, without limitation, and are preferably selected from the group consisting of PD-1 (Programmed Death 1, interchangeable used herein and equivalent to PD1)), PD-L1, PD-L2, CTLA-4/B7-1/CD152 (Cytotoxic T-Lymphocyte-Associated protein 4), CD137/4-1BB, 4-1BBL/CD137L, TIM-3 (T-cell Immunoglobulin domain and Mucin domain 3), LAG3, By-He, H4, HAVCR2, ID01, CD40/TNFRSF5, CD40L/CD154/TNFSF5, OX40/CD134, OX-40L/TNFSF4/CD252, GITR (Glucocorticoid-Induced TNFR family Related gene)/TNFRSF18, GITR ligand/TNFSF18, ICOS/AILIMI/CD278, ICOS ligand/B7-H2, CD122, CD155/PVR, CD226/DNAM-1, CD27, HVEM/TNFRSF14, TNFSF14/LIGHT/CD258, CD70/CD27L/TNFSF7, CD28/TP44, CD80/B7-1, CD86/B7-2, A2AR, KIR (Killer-cell Immunoglobulin-like Receptor), NOX2/nicotinamide adenine dinucleotide phosphate NADPH oxidase isoform 2, SIGLEC7 (Sialic acid-binding immunoglobulin-type lectin 7)/CD328, SIGLEC9 (Sialic acid-binding immunoglobulin-type lectin 9)/CD329, CD80/B7-1, CD86/B7-2, B7-H3/CD276, VTCN1/B7-H4/B7S1/7x, VISTA (V-domain Ig suppressor of T cell activation)/B7-H5/GI24, LAG3/CD223/Lymphocyte activation gene 3, indole amine 2,3-dioxygenase-dioxygenase/IDO, TDO/tryptophan 2,3-dioxygenase, Galectin-/LGALS9, TIM-3/HAVCR2, TIGIT/VSTM3, HVEM (Herpesvirus Entry Mediator)/TNFRSF14, BTLA (B and T Lymphocyte Attenuator)/CD272, CD160, CEACAM1/CD66a, indole amine, SIRP, alpha/CD172a, CD47, CD48/SLAMF2, CD30, CD30L, TMIGD2, HHLA2, TL1A, DR3, LTβR, TNF, TNFR2 and 2B4/CD244.

In a preferred embodiment, said immune checkpoint protein is a protein of the B7-CD28 family or TNFR family. In a preferred embodiment, said immune checkpoint protein is a T cell-associated checkpoint inhibitor or a non-T-cell associated checkpoint inhibitor.

In a preferred embodiment, said immune checkpoint protein is selected from the group consisting of CD137/4-1BB, 4-1BBL/CD137L, CD40/TNFRSF5, CD40L/CD154/TNFSF5, OX40/CD134, OX-40L/TNFSF4/CD252, GITR/TNFRSF18, GITR ligand/TNFSF18, ICOS (Inducible T-cell Co-stimulator)/AILIMI/CD278, ICOS ligand/B7-H2, CD122, A2AR (Adenosine A2A receptor), KIR, NOX2, SIGLEC7/CD328, SIGLEC9/CD329, PD-1, PD-L1, PD-L2, CTLA-4, CD80/B7-1, CD86/B7-2, B7-H3/CD276, B7-H4/B7S1/7x, VISTA/B7-H5/GI24, LAG3/CD223/Lymphocyte activation gene 3, 2,3-dioxygenase/IDO, Galectin-/LGALS9, TIM-3/HAVCR2, and TIGIT/VSTM3. In a further preferred embodiment, said immune checkpoint protein is selected from the group consisting of CD137/4-1BB, 4-1BBL/CD137L, CD40/TNFRSF5, CD40L/CD154/TNFSF5, OX40/CD134, OX-40L/TNFSF4/CD252, GITR/TNFRSF18, GITR ligand/TNFSF18, ICOS/AILIM/CD278, ICOS ligand/B7-H2, PD-1, PD-L1, PD-L2, CTLA-4, CD80/B7-1, CD86/B7-2, B7-H3/CD276, B7-H4/B7S1/7x, VISTA/B7-H5/GI24, LAG3/CD223/Lymphocyte activation gene 3, Galectin-9/LGALS9, TIM-3/HAVCR2, and TIGIT/VSTM3. In a further preferred embodiment, said immune checkpoint protein is selected from the group consisting of PD-1, PD-L1, PD-L2, CTLA-4, B7-1, B7-2, 4-1BB, 4-1BB ligand (4-1BBL), TIGIT, LAG3, TIM3, B7-H3, B7-H4, VISTA, CCR4, GITR ligand, GITR, OX40, OX-40L, ICOS, ICOS ligand CD40 and CD40 ligand. In a further preferred embodiment, said immune checkpoint protein is selected from the group consisting of PD-1, PD-L1, PD-L2, CTLA-4, B7-1, B7-2, 4-1BB, 4-1BB ligand, TIGIT, LAG3, TIM3, GITR, GITR ligand, CD40, CD40L, OX40, OX-40L, ICOS, and ICOS ligand. In a further preferred embodiment, said immune checkpoint protein is selected from the group consisting of PD-1, PD-L1, PD-L2, CTLA-4, B7-1, B7-2, 4-1BB, TIGIT, LAG3, TIM-3, GITR, CD40, OX40, and ICOS.

In a further preferred embodiment, said immune checkpoint protein is selected from the group consisting of PD-1, PD-L1, PD-L2, CTLA-4, B7-1, B7-2, 4-1BB, TIGIT, LAG3, TIM3, B7-H3, B7-H4, VISTA, CCR4, GITR, OX40, OX-40L, ICOS, and CD40. In a further preferred embodiment, said immune checkpoint protein is selected from the group consisting of PD-1, PD-L1, PD-L2, CTLA-4, B7-1, B7-2, 4-1BB, TIGIT, LAG3, TIM3, GITR, CD40, OX40, and ICOS. In a further preferred embodiment, said immune checkpoint protein is selected from the group consisting of PD-1, PD-L1, PD-L2, CTLA-4, 4-1BB, TIGIT, LAG3, TIM3, B7-H3, B7-H4, VISTA, CCR4, GITR, OX40, OX-40L, ICOS, and CD40. In a further preferred embodiment, said immune checkpoint protein is selected from the group consisting of PD-1, PD-L1, PD-L2, CTLA-4, 4-1BB, TIGIT, LAG3, TIM3, GITR, CD40, OX40, and ICOS.

In a further preferred embodiment, said immune checkpoint protein is selected from the group consisting of PD-1, PD-L1, PD-L2, CTLA-4, 4-1BB, LAG3, TIM3, GITR, CD40, OX40, and ICOS. In a further preferred embodiment, said immune checkpoint protein is selected from the group consisting of PD-1, PD-L1, PD-L2, CTLA-4, 4-1BB, GITR, CD40, OX40, and ICOS. In a further preferred embodiment, said immune checkpoint protein is selected from the group consisting of PD-1, PD-L1, PD-L24-1BB, LAG3, TIM3, GITR, CD40, OX40, and ICOS. In a further preferred embodiment, said immune checkpoint protein is selected from the group consisting of PD-1, PD-L1, PD-L2, CTLA-4, 4-1BB, LAG3, TIM3, GITR, CD40, and OX40. In a further preferred embodiment, said immune checkpoint protein is selected from the group consisting of PD-1, PD-L1, PD-L2, 4-1BB, LAG3, TIM3, GITR, CD40, and OX40.

In a further preferred embodiment, said immune checkpoint protein is selected from the group consisting of PD-1, PD-L1, PD-L2, 4-1BB, GITR, CD40, and OX40. In a further preferred embodiment, said immune checkpoint protein is selected from the group consisting of PD-1, PD-L1, PD-L2, CTLA-4, 4-1BB, GITR, CD40, and OX40. In a further preferred embodiment, said immune checkpoint protein is selected from the group consisting of PD-1, PD-L1, PD-L2, 4-1BB, GITR, CD40, and OX40. In a further preferred embodiment, said immune checkpoint protein is selected from the group consisting of PD-1, PD-L1, PD-L2, 4-1BB, GITR, and OX40. In a further preferred embodiment, said immune checkpoint protein is selected from the group consisting of PD-1, PD-L1, PD-L2, 4-1BB, and OX40.

In a further preferred embodiment, said immune checkpoint protein is selected from the group consisting of PD-1, PD-L1, PD-L2, 4-1BB, LAG3, and TIM3. In a further preferred embodiment, said immune checkpoint protein is selected from the group consisting of PD-1, PD-L1, PD-L2, CTLA-4, 4-1BB, LAG3, and TIM3.

In a further preferred embodiment, said immune checkpoint protein is selected from the group consisting of PD-1, PD-L1, PD-L2, CTLA-4, 4-1BB, TIGIT, LAG3, TIM-3, GITR, and ICOS. In a further preferred embodiment, said immune checkpoint protein is selected from the group consisting of PD-1, PD-L1, PD-L2, CTLA-4, 4-1BB, LAG3, TIGIT, TIM-3 and GITR. In a further preferred embodiment, said immune checkpoint protein is selected from the group consisting of PD-1, CTLA-4, 4-1BB, LAG3, TIGIT, TIM-3, GITR, and ICOS. In a further preferred embodiment, said immune checkpoint protein is selected from the group consisting of PD-1, PD-L1, PD-L2, CTLA-4, and 4-1BB. In a further preferred embodiment, said immune checkpoint protein is selected from the group consisting of PD-1, PD-L1, PD-L2, and 4-1BB. In a further preferred embodiment, said immune checkpoint protein is PD-1 or 4-1BB. In a further preferred embodiment, said immune checkpoint protein is PD-1. In another preferred embodiment, said immune checkpoint protein is PD-1, PD-L1, or PD-L2. In a further preferred embodiment, said immune checkpoint protein is PD-1, or PD-L1. In a further preferred embodiment, said immune checkpoint protein is PD-1. In a further preferred embodiment, said immune checkpoint protein is PD-L1. In a further preferred embodiment, said immune checkpoint protein is PD-L2. In a further preferred embodiment, said immune checkpoint protein is 4-1BB. In another preferred embodiment, said immune checkpoint protein is CTLA-4.

As used herein, the term “antibody” refers to immunoglobulin molecules and immunologically active portions of immunoglobulin molecules, i.e., molecules that contain an antigen binding site that selectively binds an antigen wherein the antigen comprises haptens, epitopes, receptors or ligands or parts thereof. As such, the term “antibody” encompasses not only whole antibody molecules, but also antibody fragments as well as variants (including derivatives), antibody fragments and fusion proteins. The term “antibody” also refers to antibodies comprised of two immunoglobulin heavy chains and two immunoglobulin light chains as well as a variety of forms including full length antibodies and portions thereof; including, for example, an immunoglobulin molecule, a monoclonal antibody, a chimeric antibody, a CDR-grafted antibody, a humanized antibody, a Fab, a Fab′, a F(ab′)2, a Fv, a disulfide linked Fv, a scFv, a single domain antibody (dAb), a diabody, naked antibodies, antibody-drug conjugates and bi- or tri-specific antibodies, an anti-idiotypic antibody, anticalin and a functionally active epitope-binding fragment thereof. In a particularly preferred embodiment, said antibody capable of modulating an immune checkpoint protein is a monoclonal antibody, a humanized antibody or a full human antibody. The term “antibody capable of modulating an immune checkpoint protein” as used herein refers to antibodies that modulate activity of checkpoint receptors, counter-receptors or bound and soluble ligands thereof.

As used herein, the term “anti- . . . ” relates to an antibody selectively binding to a target mentioned after the term “anti-”.

As used herein, the term “affibody” refers to proteins engineered to bind to target proteins or peptides with high affinity, imitating monoclonal antibodies, and are therefore a member of the family of antibody mimetics. Preferably the affibody has a high affinity binding domain derived from protein A. HER2 is the target of a HER2 affibody.

In a preferred embodiment, said HER2 affibody comprises an affibody selected from the group consisting of ZHER2:2891, ABY-025, ZHER2:342, and ZHER2:2395, preferably ZHER2:2891.

Preferably, said antibody capable of modulating the immune checkpoint protein is a monoclonal antibody, chimerized antibody, humanized antibody, human antibody, a fusion protein or a combination thereof.

In a further aspect, the invention provides for a kit-of-parts comprising (a) a polyplex comprising a double stranded RNA (dsRNA) and a polymeric conjugate, wherein said polymeric conjugate comprises a polyethyleneimine (PEI), one or more polyethylene glycol (PEG) moieties and one or more targeting moieties, wherein said PEI is covalently bound to one or more PEG moieties, and each of said one or more PEG moieties is linked to one of said one or more targeting moieties, and wherein each of said one or more targeting moieties is capable of binding to a cancer antigen; and (b) at least one antibody. In again further aspect, the present invention provides for a composition comprising (a) a polyplex comprising a double stranded RNA (dsRNA) and a polymeric conjugate, wherein said polymeric conjugate comprises a polyethyleneimine (PEI), one or more polyethylene glycol (PEG) moieties and one or more targeting moieties, wherein said PEI is covalently bound to one or more PEG moieties, and each of said one or more PEG moieties is linked to one of said one or more targeting moieties, and wherein each of said one or more targeting moieties is capable of binding to a cancer antigen; and (b) at least one antibody. In a preferred embodiment, said antibody is selected from the group consisting of anti-CD137/4-1BB, anti-4-1BBL/CD137L, CD40/TNFRSF5, anti-CD40L/CD154/TNFSF5, anti-OX40/CD134, anti-OX-40L/TNFSF4/CD252, anti-GITR/TNFRSF18, anti-GITR ligand/TNFSF18, anti-ICOS (Inducible T-cell Co-stimulator)/AILIM/CD278, anti-ICOS ligand/B7-H2, anti-CD122, anti-A2AR (Adenosine A2A receptor), anti-KIR, anti-NOX2, anti-SIGLEC7/CD328, anti-SIGLEC9/CD329, anti-PD-1, anti-PD-L1, anti-PD-L2, anti-CTLA-4, anti-CD80/B7-1, anti-CD86/B7-2, anti-B7-H3/CD276, anti-B7-H4/B7S1/7x, anti-VISTA/B7-H5/GI24, anti-LAG3/CD223/Lymphocyte activation gene 3, anti-2,3-dioxygenase/IDO, anti-Galectin-/LGALS9, anti-TIM-3/HAVCR2, and anti-TIGIT/VSTM3. In a further preferred embodiment, said antibody is selected from the group consisting of anti-CD137/4-1BB, anti-4-1BBL/CD137L, anti-CD40/TNFRSF5, anti-CD40L/CD154/TNFSF5, anti-OX40/CD134, anti-OX-40L/TNFSF4/CD252, anti-GITR/TNFRSF18, anti-GITR ligand/TNFSF18, anti-ICOS/AILIM/CD278, anti-ICOS ligand/B7-H2, anti-PD-1, anti-PD-L1, anti-PD-L2, anti-CTLA-4, anti-CD80/B7-1, anti-CD86/B7-2, anti-B7-H3/CD276, anti-B7-H4/B7S1/7x, anti-VISTA/B7-H5/GI24, anti-LAG3/CD223/Lymphocyte activation gene 3, anti-Galectin-9/LGALS9, anti-TIM-3/HAVCR2, and anti-TIGIT/VSTM3. In a further preferred embodiment, said antibody is selected from the group consisting of anti-PD-1, anti-PD-L1, anti-PD-L2, anti-CTLA-4, anti-B7-1, anti-B7-2, anti-4-1BB, anti-4-1BB ligand (4-1BBL), anti-TIGIT, anti-LAG3, anti-TIM3, anti-B7-H3, anti-B7-H4, anti-VISTA, anti-CCR4, anti-GITR ligand, anti-GITR, anti-OX40, anti-OX-40L, anti-ICOS, anti-ICOS ligand anti-CD40 and anti-CD40 ligand. In a further preferred embodiment, said antibody is selected from the group consisting of anti-PD-1, anti-PD-L1, anti-PD-L2, anti-CTLA-4, anti-B7-1, anti-B7-2, anti-4-1BB, anti-4-1BB ligand, anti-TIGIT, anti-LAG3, anti-TIM3, anti-GITR, anti-GITR ligand, anti-CD40, anti-CD40L, anti-OX40, anti-OX-40L, anti-ICOS, and anti-ICOS ligand.

In a further preferred embodiment, said antibody is selected from the group consisting of anti-PD-1, anti-PD-L1, anti-PD-L2, anti-CTLA-4, anti-B7-1, anti-B7-2, anti-4-1BB, anti-TIGIT, anti-LAG3, anti-TIM3, anti-B7-H3, anti-B7-H4, anti-VISTA, anti-CCR4, anti-GITR, anti-OX40, anti-ICOS, and anti-CD40. In a further preferred embodiment, said antibody is selected from the group consisting of anti-PD-1, anti-PD-L1, anti-PD-L2, anti-CTLA-4, anti-B7-1, anti-B7-2, anti-4-1BB, anti-TIGIT, anti-LAG3, anti-TIM3, anti-GITR, anti-CD40, anti-OX40, and anti-ICOS. In a further preferred embodiment, said antibody is selected from the group consisting of anti-PD-1, anti-PD-L1, anti-PD-L2, anti-CTLA-4, anti-4-1BB, anti-TIGIT, anti-LAG3, anti-TIM3, anti-GITR, anti-CD40, anti-OX40, and anti-ICOS. In a further preferred embodiment, said antibody is selected from the group consisting of anti-PD-1, anti-PD-L1, anti-PD-L2, anti-CTLA-4, anti-B7-1, anti-B7-2, anti-4-1BB, anti-TIGIT, anti-LAG3, anti-TIM-3, anti-GITR, anti-CD40, anti-OX40, and anti-ICOS. In a further preferred embodiment, said antibody is selected from the group consisting of anti-PD-1, anti-PD-L1, anti-PD-L2, anti-CTLA-4, anti-4-1BB, anti-TIGIT, anti-LAG3, anti-TIM-3, anti-GITR, and anti-ICOS. In a further preferred embodiment, said antibody is selected from the group consisting of anti-PD-1, anti-PD-L1, anti-PD-L2, anti-CTLA-4, anti-4-1BB, anti-LAG3, anti-TIGIT, anti-TIM-3, anti-GITR, and anti-ICOS. In a further preferred embodiment, said antibody is selected from the group consisting of anti-PD-1, anti-CTLA-4, anti-4-1BB, anti-LAG3, anti-TIGIT, anti-TIM-3, anti-GITR, and anti-ICOS. In a further preferred embodiment, said antibody is selected from the group consisting of anti-PD-1, anti-PD-L1, anti-PD-L2, anti-4-1BB, anti-GITR, anti-CD40, anti-ICOS and anti-OX40. In a further preferred embodiment, said antibody is selected from the group consisting of anti-PD-1, anti-PD-L1, anti-PD-L2, anti-4-1BB, anti-GITR, anti-CD40 and anti-OX40. In a further preferred embodiment, said antibody is selected from the group consisting of anti-PD-1, anti-PD-L1, anti-PD-L2, anti-4-1BB, anti-GITR and anti-OX40. In a further preferred embodiment, said antibody is selected from the group consisting of anti-PD-1, anti-PD-L1, anti-PD-L2, anti-4-1BB, and anti-OX40. In a further preferred embodiment, said antibody is selected from the group consisting of anti-PD-1, anti-PD-L1, anti-PD-L2, anti-CTLA-4, and anti-4-1BB. In a further preferred embodiment, said antibody is selected from the group consisting of anti-PD-1, anti-PD-L1, anti-PD-L2, and anti-4-1BB. In a further preferred embodiment, said antibody is anti-PD-1 or anti-4-1BB. In a further preferred embodiment, said antibody is anti-PD-1. In another preferred embodiment, said antibody is anti-PD-1, anti-PD-L1, or anti-PD-L2. In a further preferred embodiment, said antibody is anti-PD-1, or anti-PD-L1. In a further preferred embodiment, said antibody is anti-PD-1. In a further preferred embodiment, said antibody is anti-PD-L1. In a further preferred embodiment, said antibody is anti-PD-L2. In a further preferred embodiment, said antibody is anti-4-1BB. In another preferred embodiment said antibody is an anti-CTLA-4.

In a further preferred embodiment, said at least one antibody is selected from the group consisting of (i) anti-CD137/4-1BB, anti-4-1BBL/CD137L, anti-CD40/TNFRSF5, anti-CD40L/CD154/TNFSF5, anti-OX40/CD134, anti-OX-40L/TNFSF4/CD252, anti-GITR/TNFRSF18, anti-GITR ligand/TNFSF18, anti-ICOS/AILIM/CD278, and anti-ICOS ligand/B7-H2 or (ii) anti-PD-1, anti-PD-L1, anti-PD-L2, anti-CTLA-4, anti-CD80/B7-1, anti-CD86/B7-2, anti-B7-H3/CD276, anti-B7-H4/B7S1/7x, anti-VISTA/B7-H5/GI24, anti-LAG3/CD223/Lymphocyte activation gene 3, anti-Galectin-/LGALS9, anti-TIM-3/HAVCR2, and anti-TIGIT/VSTM3; or (iii) a mixture of at least one antibody of (i) and at least one antibody of (ii). In a further preferred embodiment, said at least one antibody is selected from the group consisting of (i) anti-CD137/4-1BB, anti-4-1BBL/CD137L, anti-GITR/TNFRSF18, anti-GITR ligand/TNFSF18, anti-ICOS/AILIM/CD278, and anti-ICOS ligand/B7-H2 or (ii) anti-PD-1, anti-PD-L1, anti-PD-L2, anti-CTLA-4, anti-CD80/B7-1, anti-CD86/B7-2, anti-B7-H3/CD276, anti-B7-H4/B7S1/7x, anti-VISTA/B7-H5/GI24, anti-LAG3/CD223/Lymphocyte activation gene 3, anti-Galectin-/LGALS9, anti-TIM-3/HAVCR2, and anti-TIGIT/VSTM3; or (iii) a mixture of at least one antibody of (i) and at least one antibody of (ii).

In a further preferred embodiment, said at least one antibody is selected from the group consisting of (i) anti-CD137/4-1BB, anti-4-1BBL/CD137L, anti-CD40/TNFRSF5, anti-CD40L/CD154/TNFSF5, anti-OX40/CD134, anti-OX-40L/TNFSF4/CD252, anti-GITR/TNFRSF18, anti-GITR ligand/TNFSF18, anti-ICOS/AILIM/CD278, and anti-ICOS ligand/B7-H2 or (ii) anti-PD-1, anti-PD-L1, anti-PD-L2, anti-CTLA-4, anti-B7-H3/CD276, anti-B7-H4/B7S1/7x, anti-VISTA/B7-H5/GI24, anti-LAG3/CD223/Lymphocyte activation gene 3, anti-Galectin-/LGALS9, anti-TIM-3/HAVCR2, and anti-TIGIT/VSTM3; or (iii) a mixture of at least one antibody of (i) and at least one antibody of (ii). In a further preferred embodiment, said at least one antibody is selected from the group consisting of (i) anti-CD137/4-1BB, anti-4-1BBL/CD137L, anti-GITR/TNFRSF18, anti-GITR ligand/TNFSF18, anti-ICOS/AILIM/CD278, and anti-ICOS ligand/B7-H2 or (ii) anti-PD-1, anti-PD-L1, anti-PD-L2, anti-CTLA-4, anti-B7-H3/CD276, anti-B7-H4/B7S1/7x, anti-VISTA/B7-H5/GI24, anti-LAG3/CD223/Lymphocyte activation gene 3, anti-Galectin-/LGALS9, anti-TIM-3/HAVCR2, and anti-TIGIT/VSTM3; or (iii) a mixture of at least one antibody of (i) and at least one antibody of (ii). In a further preferred embodiment, said at least one antibody is selected from the group consisting of (i) anti-CD137/4-1BB, anti-CD40/TNFRSF5, anti-OX40/CD134, anti-GITR/TNFRSF18, anti-ICOS/AILIM/CD278, or (ii) anti-PD-1, anti-PD-L1, anti-PD-L2, anti-CTLA-4, anti-B7-H3/CD276, anti-B7-H4/B7S1/7x, anti-VISTA/B7-H5/GI24, anti-LAG3/CD223/Lymphocyte activation gene 3, anti-Galectin-/LGALS9, anti-TIM-3/HAVCR2, and anti-TIGIT/VSTM3; or (iii) a mixture of at least one antibody of (i) and at least one antibody of (ii). In a further preferred embodiment, said at least one antibody is selected from the group consisting of (i) anti-CD137/4-1BB, anti-CD40/TNFRSF5, anti-OX40/CD134, anti-GITR/TNFRSF18, anti-ICOS/AILIM/CD278, or (ii) anti-PD-1, anti-PD-L1, anti-PD-L2, anti-CTLA-4; or (iii) a mixture of at least one antibody of (i) and at least one antibody of (ii). In a further preferred embodiment, said at least one antibody is selected from the group consisting of (i) anti-CD137/4-1BB, anti-CD40/TNFRSF5, anti-OX40/CD134, anti-GITR/TNFRSF18, anti-ICOS/AILIM/CD278, or (ii) anti-PD-1, anti-PD-L1, anti-PD-L2; or (iii) a mixture of at least one antibody of (i) and at least one antibody of (ii). In a further preferred embodiment, said at least one antibody is selected from the group consisting of (i) anti-CD137/4-1BB, anti-CD40/TNFRSF5, anti-OX40/CD134, anti-GITR/TNFRSF18, or (ii) anti-PD-1, anti-PD-L1, anti-PD-L2; or (iii) a mixture of at least one antibody of (i) and at least one antibody of (ii). In a further preferred embodiment, said at least one antibody is selected from the group consisting of (i) anti-CD137/4-1BB, anti-CD40/TNFRSF5, anti-OX40/CD134, or (ii) anti-PD-1, anti-PD-L1, anti-PD-L2; or (iii) a mixture of at least one antibody of (i) and at least one antibody of (ii). In a further preferred embodiment, said at least one antibody is selected from the group consisting of (i) anti-CD137/4-1BB, anti-OX40/CD134, or (ii) anti-PD-1, anti-PD-L1, anti-PD-L2; or (iii) a mixture of at least one antibody of (i) and at least one antibody of (ii).

In a further preferred embodiment, said at least one antibody is selected from the group consisting of (i) anti-CD137/4-1BB, anti-4-1BBL/CD137L, anti-GITR/TNFRSF18, anti-GITR ligand/TNFSF18, anti-ICOS/AILIMI/CD278, and anti-ICOS ligand/B7-H2 or (ii) anti-PD-1, anti-PD-L1, anti-PD-L2, anti-CTLA-4, anti-LAG3/CD223/Lymphocyte activation gene 3, anti-TIM-3/HAVCR2, and anti-TIGIT/VSTM3; or (iii) a mixture of at least one antibody of (i) and at least one antibody of (ii). In a further preferred embodiment, said at least one antibody is selected from the group consisting of (i) anti-4-1BB, anti-GITR, anti-OX40, anti-ICOS, and anti-CD40; or (ii) anti-PD-1, anti-PD-L1, anti-PD-L2, anti-CTLA-4, anti-TIGIT, anti-LAG3, anti-TIM3, anti-B7-H3, anti-B7-H4, anti-VISTA, anti-CCR4, and anti-GITRL; or (iii) a mixture of at least one antibody of (i) and at least one antibody of (ii). In a further preferred embodiment, said at least one antibody is selected from the group consisting of (i) anti-CD137/4-1BB, anti-4-1BBL/CD137L; or (ii) anti-PD-1, anti-PD-L1, anti-PD-L2, anti-CTLA-4; or (iii) a mixture of at least one antibody of (i) and at least one antibody of (ii). In a further preferred embodiment, said at least one antibody is selected from the group consisting of (i) anti-CD137/4-1BB, anti-4-1BBL/CD137L; or (ii) anti-PD-1, anti-PD-L1, anti-PD-L2; or (iii) a mixture of at least one antibody of (i) and at least one antibody of (ii).

In a preferred embodiment, said antibody capable of modulating an immune checkpoint protein is a bispecific antibody capable of binding to a cancer antigen and an immune checkpoint protein. Preferably, said cancer antigen is EGFR, HER2 or PSMA, more preferably said cancer antigen is EGFR or HER2, and said immune checkpoint protein is selected from the group consisting of PD-1, PD-L1, PD-L2, CTLA-4, and 4-1BB. More preferably said cancer antigen is EGFR or HER2, and said immune checkpoint protein is selected from the group consisting of PD-1, PD-L1, PD-L2 and 4-1BB. Again more preferably said cancer antigen is EGFR or HER2, and said immune checkpoint protein is PD-1 or 4-1BB. Again more preferably, said cancer antigen is HER2 or EGFR and said immune checkpoint protein is 4-1BB. In another preferred embodiment, said cancer antigen is HER2 and said immune checkpoint protein is 4-1BB. In another preferred embodiment, said cancer antigen is PSMA and said immune checkpoint protein is selected from the group consisting of PD-1, PD-L1, PD-L2, CTLA-4, and 4-1BB. In another preferred embodiment, said cancer antigen is EGFR and said immune checkpoint protein is 4-1BB. In a preferred embodiment, said antibody capable of modulating an immune checkpoint protein is a lipocalin that binds to 4-1BB and HER2.

Preferred examples of said antibody capable of modulating the immune checkpoint protein PD-1 (anti-PD-1) are human or humanized antibodies selected from the group consisting of pembrolizumab, nivolumab (known also as MDX-1106 or BMS-936558, Topalian et al., 2012. N. Eng. J. Med. 366:2443-2454, disclosed in U.S. Pat. No. 8,008,449 B2), cemiplimab, IBI308, BCD-100, PDR001, tislelizumab, camrelizumab, pidilizumab (disclosed in Rosenblatt et al., 2011, J Immunother. 34:409-18), and lambrolizumab (e.g. disclosed as hPD109A and its humanized derivatives h409All, h409A16 and h409A17 in WO2008/156712; Hamid et al., 2013, N. Engl. J. Med. 369: 134-144) and soluble PD-1 ligands including without limitation PD-L2 Fc fusion protein (also known as B7-DC-Ig or AMP-244; disclosed in Mkrtichyan M, et al., 2012, J Immunol. 189: 2338-47). More preferred examples of said antibody capable of modulating the immune checkpoint protein PD-1 (anti-PD-1) are pembrolizumab or nivolumab.

Preferred examples of said antibody capable of modulating the immune checkpoint protein PD-L1 (anti-PD-L1) are antibodies selected from the group consisting of durvalumab, avelumab, and atezolizumab, MEDI-4736 (disclosed e.g. in WO 2011/066389 A1), MPDL328 OA (disclosed e.g. in U.S. Pat. No. 8,217,149 B2) and MIH1 (Affymetrix). More preferred examples of said antibody capable of modulating the immune checkpoint protein PD-L1 (anti-PD-L1) are antibodies selected from the group consisting of durvalumab, avelumab, and atezolizumab.

Preferred examples of said antibody capable of modulating the immune checkpoint protein CTLA-4 are ipilimumab or tremelimumab, more preferably ipilimumab. Ipilimumab is a fully human CTLA-4 blocking antibody presently marketed under the name Yervoy (Bristol-Myers Squibb). A further CTLA-4 inhibitor is tremelimumab (referenced in Ribas et al., 2013, J. Clin. Oncol. 31:616-22).

A preferred example of said antibody capable of modulating the immune checkpoint protein CD27 is CDX-1127, an agonistic anti-CD27 monoclonal antibody.

Preferred examples of said antibody capable of modulating the immune checkpoint protein OX40 are MEDI0562, a humanized OX40 agonist; MEDI6469, a murine OX4 agonist; and MEDI6383, an OX40 agonist.

A preferred example of said antibody capable of modulating the immune checkpoint protein KIR is Lirilumab, a monoclonal antibody to KIR.

A preferred example of said antibody capable of modulating the immune checkpoint protein 4-1BB is urelumab. A preferred example of said antibody capable of modulating the immune checkpoint protein LAG-3 is relatlimab.

A preferred example of said antibody capable of modulating the immune checkpoint protein LAG3 is the monoclonal antibody BMS-986016.

In a further preferred embodiment, said at least one antibody capable of modulating an immune checkpoint protein is selected from the group consisting of pembrolizumab, nivolumab, cemiplimab, IBI308, BCD-100, PDR001, tislelizumab, camrelizumab, pidilizumab, lambrolizumab, h409All, h409A16, h409A17, soluble PD-1 ligands such as PD-L2 Fc fusion protein, durvalumab, avelumab, atezolizumab, MEDI-4736, MPDL328 OA, CDX-1127, MIH1, MEDI0562, MEDI6469, MEDI6383, ipilimumab, tremelimumab, relatlimab, urelumab, anti-TIGIT antibody, anti-TIM3 antibody, anti-GITR antibody and anti-ICOS antibody. In a further preferred embodiment, said at least one antibody capable of modulating an immune checkpoint protein is selected from the group consisting of pembrolizumab, nivolumab, cemiplimab, IBI308, BMS-986016, BCD-100, PDR001, tislelizumab, camrelizumab, pidilizumab, lambrolizumab, h409All, h409A16, h409A17, soluble PD-1 ligands such as PD-L2 Fc fusion protein, durvalumab, avelumab, atezolizumab, Lirilumab, MEDI-4736, MPDL328 OA, MIH1, ipilimumab, tremelimumab, relatlimab and urelumab. In a further preferred embodiment, said at least one antibody capable of modulating an immune checkpoint protein is selected from the group consisting of pembrolizumab, nivolumab, cemiplimab, IBI308, BCD-100, PDR001, tislelizumab, camrelizumab, pidilizumab, lambrolizumab, h409All, h409A16, h409A17, soluble PD-1 ligands such as PD-L2 Fc fusion protein, durvalumab, avelumab, atezolizumab, MEDI-4736, MPDL328 OA, MIH1, ipilimumab, tremelimumab, and urelumab. In a further preferred embodiment, said at least one antibody capable of modulating an immune checkpoint protein is selected from the group consisting of pembrolizumab, nivolumab, cemiplimab, IBI308, BCD-100, PDR001, tislelizumab, camrelizumab, pidilizumab, lambrolizumab, h409All, h409A16, h409A17, soluble PD-1 ligands such as PD-L2 Fc fusion protein, durvalumab, avelumab, atezolizumab, MEDI-4736, MPDL328 OA, MIH1, tremelimumab, and urelumab. In a further preferred embodiment, said at least one antibody capable of modulating an immune checkpoint protein is selected from the group consisting of pembrolizumab, nivolumab, cemiplimab, IBI308, BCD-100, PDR001, tislelizumab, camrelizumab, pidilizumab, lambrolizumab, h409All, h409A16, h409A17, soluble PD-1 ligands such as PD-L2 Fc fusion protein, durvalumab, avelumab, atezolizumab, MEDI-4736, MPDL328 OA and MIH1.

In a preferred embodiment, said at least one antibody capable of modulating an immune checkpoint protein is (i) at least one antibody capable of agonizing co-stimulatory immune checkpoint proteins (called herein checkpoint activator); (ii) at least one antibody capable of antagonizing inhibitory immune checkpoint proteins (called herein checkpoint inhibitor); or (iii) a mixture of both (i) and (ii). In a preferred embodiment, said at least one immune checkpoint modulating antibody capable of modulating an immune checkpoint protein is at least one antibody capable of agonizing co-stimulatory immune checkpoint proteins (checkpoint activator). Said checkpoint activator activates co-stimulatory immune checkpoint proteins. Checkpoint activators deliver activating signals to T cells, B cells or natural killer cells either directly by agonizing the receptors of said cell types, inhibition or blockage of inhibitory ligands or agonizing counter-receptors on antigen-presenting cells (APCs).

In a preferred embodiment, said at least one immune checkpoint modulating antibody capable of modulating an immune checkpoint protein is at least one antibody capable of antagonizing inhibitory immune checkpoint proteins (checkpoint inhibitor). Said checkpoint inhibitor disinhibits, i.e. reduces or removes inhibitory function of inhibitory immune checkpoint proteins. Checkpoint inhibitors deliver antagonizing signals to T cells, B cells or natural killer cells either directly by antagonizing the receptors of said cell types, agonizing of inhibitory ligands or antagonizing counter-receptors on antigen-presenting cells (APCs).

In another preferred embodiment, said at least one antibody capable of modulating an immune checkpoint protein is an immune checkpoint activator or inhibitor. In another preferred embodiment, said at least one antibody capable of modulating an immune checkpoint protein is an immune checkpoint activator and inhibitor.

In a further preferred embodiment, said at least one antibody capable of modulating an immune checkpoint protein is (i) at least one antibody capable of agonizing a co-stimulatory immune checkpoint protein, wherein said co-stimulatory immune checkpoint protein is selected from the group consisting of CD155/PVR, CD226/DNAM-1, CD137/4-1BB, CD40/TNFRSF5, CD40L/CD154/TNFSF5, 4-1BBL/CD137L, OX40/CD134, OX-40L/TNFSF4/CD252, CD27, CD122, HVEM/TNFRSF14, TNFSF14/LIGHT/CD258, CD70/CD27L/TNFSF7, CD28/TP44, CD30, CD30 ligand, TMIGD2, HHLA2, CD80/B7-1, CD86/B7-2, GITR/TNFRSF18, GITR ligand/TNFSF18, DR3, TL1A, CD30, CD30L, TMIGD2, HHLA2, TL1A, DR3, LTβR, TNF, TNFR2, ICOS/AILIMI/CD278, and ICOS ligand/B7-H2 or (ii) at least one antibody capable of antagonizing a co-inhibitory immune checkpoint protein, wherein said co-inhibitory immune checkpoint protein is selected from the group consisting of PD-1, PD-L1, PD-L2, CTLA-4, CD80/B7-1, CD86/B7-2, B7-H3/CD276, B7-H4/B7S1/7x, VISTA/B7-H5/GI24, HVEM/TNFRSF14, BTLA, CD160, LAG3/CD223/Lymphocyte activation gene 3, CEACAM1/CD66a, indole amine, 2,3-dioxygenase/IDO, Galectin-/LGALS9, TIM-3/HAVCR2, 2B4/CD244, SIRP, alpha/CD172a, CD47, CD48/SLAMF2, TIGIT/VSTM3, A2AR, KIR, NOX2, SIGLEC7/CD328, SIGLEC9/CD329 and, or (iii) a mixture of both. In a further preferred embodiment, said at least one antibody capable of modulating an immune checkpoint protein is (i) at least one antibody capable of agonizing a co-stimulatory immune checkpoint protein, wherein said co-stimulatory immune checkpoint protein is selected from the group consisting of CD155/PVR, CD226/DNAM-1, CD137/4-1BB, CD40/TNFRSF5, CD40L/CD154/TNFSF5, 4-1BBL/CD137L, OX40/CD134, OX-40L/TNFSF4/CD252, CD27, CD122, HVEM/TNFRSF14, TNFSF14/LIGHT/CD258, CD70/CD27L/TNFSF7, CD28/TP44, CD30, CD30 ligand, TMIGD2, HHLA2, CD80/B7-1, CD86/B7-2, GITR/TNFRSF18, GITR ligand/TNFSF18, DR3, TL1A, CD30, CD30L, TMIGD2, HHLA2, TL1A, DR3, LTβR, TNF, TNFR2, ICOS/AILIMI/CD278, and ICOS ligand/B7-H2 or (ii) at least one antibody capable of antagonizing a co-inhibitory immune checkpoint protein, wherein said co-inhibitory immune checkpoint protein is selected from the group consisting of PD-1, PD-L1, PD-L2, CTLA-4, B7-H3/CD276, B7-H4/B7S1/7x, VISTA/B7-H5/GI24, HVEM/TNFRSF14, BTLA, CD160, LAG3/CD223/Lymphocyte activation gene 3, CEACAM1/CD66a, indole amine, 2,3-dioxygenase/IDO, Galectin-/LGALS9, TIM-3/HAVCR2, 2B4/CD244, SIRP, alpha/CD172a, CD47, CD48/SLAMF2, TIGIT/VSTM3, A2AR, KIR, NOX2, SIGLEC7/CD328, SIGLEC9/CD329 and, or (iii) a mixture of both. In a further preferred embodiment, said at least one antibody capable of modulating an immune checkpoint protein is (i) at least one antibody capable of agonizing a co-stimulatory immune checkpoint protein, wherein said co-stimulatory immune checkpoint protein is selected from the group consisting of CD155/PVR, CD226/DNAM-1, CD137/4-1BB, CD40/TNFRSF5, CD40L/CD154/TNFSF5, 4-1BBL/CD137L, OX40/CD134, OX-40L/TNFSF4/CD252, CD27, HVEM/TNFRSF14, TNFSF14/LIGHT/CD258, CD70/CD27L/TNFSF7, CD28/TP44, CD80/B7-1, CD86/B7-2, GITR/TNFRSF18, GITR ligand/TNFSF18, CD30, CD30L, TMIGD2, HHLA2, TL1A, DR3, LTβR, TNF, TNFR2, ICOS/AILIMI/CD278, and ICOS ligand/B7-H2 or (ii) at least one antibody capable of antagonizing a co-inhibitory immune checkpoint protein, wherein said co-inhibitory immune checkpoint protein is selected from the group consisting of PD-1, PD-L1, PD-L2, CTLA-4, CD80/B7-1, CD86/B7-2, B7-H3/CD276, B7-H4/B7S1/7x, VISTA/B7-H5/GI24, HVEM/TNFRSF14, BTLA, CD160, LAG3/CD223/Lymphocyte activation gene 3, CEACAM1/CD66a, indole amine, Galectin-/LGALS9, TIM-3/HAVCR2, 2B4/CD244, SIRP, alpha/CD172a, CD47, CD48/SLAMF2, and TIGIT/VSTM3. In a further preferred embodiment, said at least one antibody capable of modulating an immune checkpoint protein is (i) at least one antibody capable of agonizing a co-stimulatory immune checkpoint protein, wherein said co-stimulatory immune checkpoint protein is selected from the group consisting of CD155/PVR, CD226/DNAM-1, CD137/4-1BB, CD40/TNFRSF5, CD40L/CD154/TNFSF5, 4-1BBL/CD137L, OX40/CD134, OX-40L/TNFSF4/CD252, CD27, HVEM/TNFRSF14, TNFSF14/LIGHT/CD258, CD70/CD27L/TNFSF7, CD28/TP44, CD80/B7-1, CD86/B7-2, GITR/TNFRSF18, GITR ligand/TNFSF18, CD30, CD30L, TMIGD2, HHLA2, TL1A, DR3, LTβR, TNF, TNFR2, ICOS/AILIM/CD278, and ICOS ligand/B7-H2 or (ii) at least one antibody capable of antagonizing a co-inhibitory immune checkpoint protein, wherein said co-inhibitory immune checkpoint protein is selected from the group consisting of PD-1, PD-L1, PD-L2, CTLA-4, B7-H3/CD276, B7-H4/B7S1/7x, VISTA/B7-H5/GI24, HVEM/TNFRSF14, BTLA, CD160, LAG3/CD223/Lymphocyte activation gene 3, CEACAM1/CD66a, indole amine, Galectin-/LGALS9, TIM-3/HAVCR2, 2B4/CD244, SIRP, alpha/CD172a, CD47, CD48/SLAMF2, and TIGIT/VSTM3. In a further preferred embodiment, said at least one antibody capable of modulating an immune checkpoint protein is (i) at least one antibody capable of agonizing a co-stimulatory immune checkpoint protein, wherein said co-stimulatory immune checkpoint protein is selected from the group consisting of CD137/4-1BB, 4-1BBL/CD137L, CD40/TNFRSF5, CD40L/CD154/TNFSF5, OX40/CD134, OX-40L/TNFSF4/CD252, GITR/TNFRSF18, GITR ligand/TNFSF18, ICOS/AILIM/CD278, and ICOS ligand/B7-H2 or (ii) at least one antibody capable of antagonizing a co-inhibitory immune checkpoint protein, wherein said co-inhibitory immune checkpoint protein is selected from the group consisting of PD-1, PD-L1, PD-L2, CTLA-4, CD80/B7-1, CD86/B7-2, B7-H3/CD276, B7-H4/B7S1/7x, VISTA/B7-H5/GI24, LAG3/CD223/Lymphocyte activation gene 3, Galectin-/LGALS9, TIM-3/HAVCR2, and TIGIT/VSTM3; or (iii) a mixture of both. In a further preferred embodiment, said at least one antibody capable of modulating an immune checkpoint protein is (i) at least one antibody capable of agonizing a co-stimulatory immune checkpoint protein, wherein said co-stimulatory immune checkpoint protein is selected from the group consisting of CD137/4-1BB, 4-1BBL/CD137L, GITR/TNFRSF18, GITR ligand/TNFSF18, ICOS/AILIM/CD278, and ICOS ligand/B7-H2 or (ii) at least one antibody capable of antagonizing a co-inhibitory immune checkpoint protein, wherein said co-inhibitory immune checkpoint protein is selected from the group consisting of PD-1, PD-L1, PD-L2, CTLA-4, CD80/B7-1, CD86/B7-2, B7-H3/CD276, B7-H4/B7S1/7x, VISTA/B7-H5/GI24, LAG3/CD223/Lymphocyte activation gene 3, Galectin-/LGALS9, TIM-3/HAVCR2, and TIGIT/VSTM3; or (iii) a mixture of both. In a further preferred embodiment, said at least one antibody capable of modulating an immune checkpoint protein is (i) at least one antibody capable of agonizing a co-stimulatory immune checkpoint protein, wherein said co-stimulatory immune checkpoint protein is selected from the group consisting of CD137/4-1BB, 4-1BBL/CD137L, CD40/TNFRSF5, CD40L/CD154/TNFSF5, OX40/CD134, OX-40L/TNFSF4/CD252, GITR/TNFRSF18, GITR ligand/TNFSF18, ICOS/AILIM/CD278, and ICOS ligand/B7-H2 or (ii) at least one antibody capable of antagonizing a co-inhibitory immune checkpoint protein, wherein said co-inhibitory immune checkpoint protein is selected from the group consisting of PD-1, PD-L1, PD-L2, CTLA-4, B7-H3/CD276, B7-H4/B7S1/7x, VISTA/B7-H5/GI24, LAG3/CD223/Lymphocyte activation gene 3, Galectin-/LGALS9, TIM-3/HAVCR2, and TIGIT/VSTM3; or (iii) a mixture of both. In a further preferred embodiment, said at least one antibody capable of modulating an immune checkpoint protein is (i) at least one antibody capable of agonizing a co-stimulatory immune checkpoint protein, wherein said co-stimulatory immune checkpoint protein is selected from the group consisting of CD137/4-1BB, 4-1BBL/CD137L, GITR/TNFRSF18, GITR ligand/TNFSF18, ICOS/AILIM/CD278, and ICOS ligand/B7-H2 or (ii) at least one antibody capable of antagonizing a co-inhibitory immune checkpoint protein, wherein said co-inhibitory immune checkpoint protein is selected from the group consisting of PD-1, PD-L1, PD-L2, CTLA-4, B7-H3/CD276, B7-H4/B7S1/7x, VISTA/B7-H5/GI24, LAG3/CD223/Lymphocyte activation gene 3, Galectin-/LGALS9, TIM-3/HAVCR2, and TIGIT/VSTM3; or (iii) a mixture of both.

In a preferred embodiment of the composition or the kit-of-parts of the invention, said at least one antibody capable of modulating an immune checkpoint protein is (i) at least one antibody capable of agonizing a co-stimulatory immune checkpoint protein, wherein said co-stimulatory immune checkpoint protein is selected from the group consisting of 4-1BB, 4-1BB ligand (4-1BBL), CD40, CD40 ligand (CD40L), OX40, OX-40 ligand (OX-40L), GITR, GITR ligand (GITRL), ICOS and ICOS ligand (ICOSL); or (ii) at least one antibody capable of antagonizing a co-inhibitory immune checkpoint protein, wherein said co-inhibitory immune checkpoint protein is selected from the group consisting of PD-1, PD-L1, PD-L2, CTLA-4, B7-1, B7-2, B7-H3, B7-H4, VISTA, LAG-3, Galectin-9, TIM-3, and TIGIT; or (iii) a mixture of at least one antibody of (i) and at least one antibody of (ii).

In a preferred embodiment of the composition or the kit-of-parts of the invention, said at least one antibody capable of modulating an immune checkpoint protein is (i) at least one antibody capable of agonizing a co-stimulatory immune checkpoint protein, wherein said co-stimulatory immune checkpoint protein is selected from the group consisting of 4-1BB, 4-1BB ligand (4-1BBL), CD40, CD40 ligand (CD40L), OX40, OX-40 ligand (OX-40L), GITR, GITR ligand (GITRL), ICOS and ICOS ligand (ICOSL); or (ii) at least one antibody capable of antagonizing a co-inhibitory immune checkpoint protein, wherein said co-inhibitory immune checkpoint protein is selected from the group consisting of PD-1, PD-L1, PD-L2, CTLA-4, B7-H3, B7-H4, VISTA, LAG-3, Galectin-9, TIM-3, and TIGIT; or (iii) a mixture of at least one antibody of (i) and at least one antibody of (ii). AVCR2, and TIGIT/VSTM3; or (iii) a mixture of both. In a further preferred embodiment of the composition or the kit-of-parts of the invention, said at least one antibody capable of modulating an immune checkpoint protein is (i) at least one antibody capable of agonizing a co-stimulatory immune checkpoint protein, wherein said co-stimulatory immune checkpoint protein is selected from the group consisting of 4-1BB, CD40, OX40, GITR, and ICOS; or (ii) at least one antibody capable of antagonizing a co-inhibitory immune checkpoint protein, wherein said co-inhibitory immune checkpoint protein is selected from the group consisting of PD-1, PD-L1, PD-L2, CTLA-4, B7-1, B7-2, B7-H3, B7-H4, VISTA, LAG-3, Galectin-9, TIM-3, and TIGIT; or (iii) a mixture of at least one antibody of (i) and at least one antibody of (ii). In a further preferred embodiment of the composition or the kit-of-parts of the invention, said at least one antibody capable of modulating an immune checkpoint protein is (i) at least one antibody capable of agonizing a co-stimulatory immune checkpoint protein, wherein said co-stimulatory immune checkpoint protein is selected from the group consisting of 4-1BB, CD40, OX40, GITR, and ICOS; or (ii) at least one antibody capable of antagonizing a co-inhibitory immune checkpoint protein, wherein said co-inhibitory immune checkpoint protein is selected from the group consisting of PD-1, PD-L1, PD-L2, CTLA-4, B7-H3, B7-H4, VISTA, LAG-3, Galectin-9, TIM-3, and TIGIT; or (iii) a mixture of at least one antibody of (i) and at least one antibody of (ii).

In a further preferred embodiment of the composition or the kit-of-parts of the invention, said at least one antibody capable of modulating an immune checkpoint protein is (i) at least one antibody capable of agonizing a co-stimulatory immune checkpoint protein, wherein said co-stimulatory immune checkpoint protein is selected from the group consisting of 4-1BB, CD40, OX40, and GITR; or (ii) at least one antibody capable of antagonizing a co-inhibitory immune checkpoint protein, wherein said co-inhibitory immune checkpoint protein is selected from the group consisting of PD-1, PD-L1, PD-L2, CTLA-4, B7-H3, B7-H4, VISTA, LAG-3, Galectin-9, TIM-3, and TIGIT; or (iii) a mixture of at least one antibody of (i) and at least one antibody of (ii).

In a further preferred embodiment of the composition or the kit-of-parts of the invention, said at least one antibody capable of modulating an immune checkpoint protein is (i) at least one antibody capable of agonizing a co-stimulatory immune checkpoint protein, wherein said co-stimulatory immune checkpoint protein is selected from the group consisting of 4-1BB, CD40, OX40, ICOS and GITR; or (ii) at least one antibody capable of antagonizing a co-inhibitory immune checkpoint protein, wherein said co-inhibitory immune checkpoint protein is selected from the group consisting of PD-1, PD-L1, PD-L2, CTLA-4; or (iii) a mixture of at least one antibody of (i) and at least one antibody of (ii).

In a further preferred embodiment of the composition or the kit-of-parts of the invention, said at least one antibody capable of modulating an immune checkpoint protein is (i) at least one antibody capable of agonizing a co-stimulatory immune checkpoint protein, wherein said co-stimulatory immune checkpoint protein is selected from the group consisting of 4-1BB, CD40, OX40, and GITR; or (ii) at least one antibody capable of antagonizing a co-inhibitory immune checkpoint protein, wherein said co-inhibitory immune checkpoint protein is selected from the group consisting of PD-1, PD-L1, PD-L2; or (iii) a mixture of at least one antibody of (i) and at least one antibody of (ii). In a further preferred embodiment of the composition or the kit-of-parts of the invention, said at least one antibody capable of modulating an immune checkpoint protein is (i) at least one antibody capable of agonizing a co-stimulatory immune checkpoint protein, wherein said co-stimulatory immune checkpoint protein is selected from the group consisting of 4-1BB, OX40, and GITR; or (ii) at least one antibody capable of antagonizing a co-inhibitory immune checkpoint protein, wherein said co-inhibitory immune checkpoint protein is selected from the group consisting of PD-1, PD-L1, PD-L2; or (iii) a mixture of at least one antibody of (i) and at least one antibody of (ii). In a further preferred embodiment of the composition or the kit-of-parts of the invention, said at least one antibody capable of modulating an immune checkpoint protein is (i) at least one antibody capable of agonizing a co-stimulatory immune checkpoint protein, wherein said co-stimulatory immune checkpoint protein is selected from the group consisting of 4-1BB, and OX40; or (ii) at least one antibody capable of antagonizing a co-inhibitory immune checkpoint protein, wherein said co-inhibitory immune checkpoint protein is selected from the group consisting of PD-1, PD-L1, PD-L2; or (iii) a mixture of at least one antibody of (i) and at least one antibody of (ii).

In a further preferred embodiment, said at least one antibody capable of modulating an immune checkpoint protein is (i) at least one antibody capable of agonizing a co-stimulatory immune checkpoint protein, wherein said co-stimulatory immune checkpoint protein is selected from the group consisting of CD137/4-1BB, 4-1BBL/CD137L, GITR/TNFRSF18, GITR ligand/TNFSF18, ICOS/AILIM/CD278, and ICOS ligand/B7-H2 or (ii) at least one antibody capable of antagonizing a co-inhibitory immune checkpoint protein, wherein said co-inhibitory immune checkpoint protein is selected from the group consisting of PD-1, PD-L1, PD-L2, CTLA-4, LAG3/CD223/Lymphocyte activation gene 3, TIM-3/HAVCR2, and TIGIT/VSTM3; or (iii) a mixture of both. In a further preferred embodiment, said at least one antibody capable of modulating an immune checkpoint protein is (i) at least one antibody capable of agonizing a co-stimulatory immune checkpoint protein, wherein said co-stimulatory immune checkpoint protein is selected from the group consisting of 4-1BB, GITR, OX40, ICOS, and CD40; or (ii) at least one antibody capable of antagonizing a co-inhibitory immune checkpoint protein, wherein said co-inhibitory immune checkpoint protein is selected from the group consisting of PD-1, PD-L1, PD-L2, CTLA-4, TIGIT, LAG3, TIM3, B7-H3, B7-H4, VISTA, CCR4, and GITRL; or (iii) a mixture of both. In a further preferred embodiment, said at least one antibody capable of modulating an immune checkpoint protein is (i) at least one antibody capable of agonizing a co-stimulatory immune checkpoint protein, wherein said co-stimulatory immune checkpoint protein is selected from the group consisting of CD137/4-1BB, 4-1BBL/CD137L; or (ii) at least one antibody capable of antagonizing a co-inhibitory immune checkpoint protein, wherein said co-inhibitory immune checkpoint protein is selected from the group consisting of PD-1, PD-L1, PD-L2, CTLA-4; or (iii) a mixture of both. In a further preferred embodiment, said at least one antibody capable of modulating an immune checkpoint protein is (i) at least one antibody capable of agonizing a co-stimulatory immune checkpoint protein, wherein said co-stimulatory immune checkpoint protein is selected from the group consisting of CD137/4-1BB, 4-1BBL/CD137L; or (ii) at least one antibody capable of antagonizing a co-inhibitory immune checkpoint protein, wherein said co-inhibitory immune checkpoint protein is selected from the group consisting of PD-1, PD-L1, PD-L2; or (iii) a mixture of both.

In a further preferred embodiment, said at least one antibody capable of modulating an immune checkpoint protein is (i) at least one antibody capable of agonizing a co-stimulatory immune checkpoint protein, wherein said co-stimulatory immune checkpoint protein is selected from the group consisting of CD155/PVR, CD226/DNAM-1, CD137/4-1BB, CD40/TNFRSF5, CD40L/CD154/TNFSF5, 4-1BBL/CD137L, OX40/CD134, OX-40L/TNFSF4/CD252, CD27, HVEM/TNFRSF14, TNFSF14/LIGHT/CD258, CD70/CD27L/TNFSF7, CD28/TP44, CD30, CD30 ligand (L), TMIGD2, HHLA2, CD80/B7-1, CD86/B7-2, GITR/TNFRSF18, GITR ligand/TNFSF18, DR3, TL1A, CD30, CD30L, TMIGD2, HHLA2, TL1A, DR3, LTβR, TNF, TNFR2, ICOS/AILIMI/CD278, and ICOS ligand/B7-H2. In a further preferred embodiment, said at least one antibody capable of modulating an immune checkpoint protein is (i) at least one antibody capable of agonizing a co-stimulatory immune checkpoint protein, wherein said co-stimulatory immune checkpoint protein is selected from the group consisting of CD137/4-1BB, 4-1BBL/CD137L, CD40/TNFRSF5, CD40L/CD154/TNFSF5, OX40/CD134, OX-40L/TNFSF4/CD252, GITR/TNFRSF18, GITR ligand/TNFSF18, ICOS/AILIM/CD278, and ICOS ligand ligand/B7-H2. In a further preferred embodiment, said at least one antibody capable of modulating an immune checkpoint protein is (i) at least one antibody capable of agonizing a co-stimulatory immune checkpoint protein, wherein said co-stimulatory immune checkpoint protein is selected from the group consisting of CD137/4-1BB, 4-1BBL/CD137L, GITR/TNFRSF18, GITR ligand/TNFSF18, ICOS/AILIM/CD278, and ICOS ligand/B7-H2. In a further preferred embodiment, said at least one antibody capable of modulating an immune checkpoint protein is (i) at least one antibody capable of agonizing a co-stimulatory immune checkpoint protein, wherein said co-stimulatory immune checkpoint protein is selected from the group consisting of 4-1BB, GITR, OX40, ICOS, and CD40. In a further preferred embodiment, said at least one antibody capable of modulating an immune checkpoint protein is (i) at least one antibody capable of agonizing a co-stimulatory immune checkpoint protein, wherein said co-stimulatory immune checkpoint protein is selected from the group consisting of CD137/4-1BB, 4-1BBL/CD137L, GITR/TNFRSF18, GITR ligand/TNFSF18, ICOS/AILIM/CD278, and ICOS ligand/B7-H2. In a further preferred embodiment, said at least one antibody capable of modulating an immune checkpoint protein is (i) at least one antibody capable of agonizing a co-stimulatory immune checkpoint protein, wherein said co-stimulatory immune checkpoint protein is CD137/4-1BB or 4-1BBL/CD137L. In a further preferred embodiment, said at least one antibody capable of modulating an immune checkpoint protein is (i) at least one antibody capable of agonizing a co-stimulatory immune checkpoint protein, wherein said co-stimulatory immune checkpoint protein is CD137/4-1BB.

In a further preferred embodiment, said at least one antibody capable of modulating an immune checkpoint protein is (i) at least one antibody capable of agonizing a co-stimulatory immune checkpoint protein, wherein said agonizing antibody is selected from the group consisting of anti-4-1BB, anti-GITR, anti-OX40, anti-ICOS, and anti-CD40. In a further preferred embodiment, said at least one antibody capable of modulating an immune checkpoint protein is (i) at least one antibody capable of agonizing a co-stimulatory immune checkpoint protein, wherein said agonizing antibody is selected from the group consisting of anti-CD137/4-1BB, anti-4-1BBL/CD137L, anti-GITR/TNFRSF18, anti-GITR ligand/TNFSF18, anti-ICOS/AILIM/CD278, and anti-ICOS ligand/B7-H2. In a further preferred embodiment, said at least one antibody capable of modulating an immune checkpoint protein is (i) at least one antibody capable of agonizing a co-stimulatory immune checkpoint protein, wherein said agonizing antibody is anti-CD137/4-1BB or anti-4-1BBL/CD137L. In a further preferred embodiment, said at least one antibody capable of modulating an immune checkpoint protein is (i) at least one antibody capable of agonizing a co-stimulatory immune checkpoint protein, wherein said agonizing antibody is anti-CD137/4-1BB.

In a further preferred embodiment, said at least one antibody capable of modulating an immune checkpoint protein is (ii) at least one antibody capable of antagonizing a co-inhibitory immune checkpoint protein, wherein said co-inhibitory immune checkpoint protein is selected from the group consisting of PD-1, PD-L1, PD-L2, CTLA-4, CD80/B7-1, CD86/B7-2, B7-H3/CD276, B7-H4/B7S1/7x, VISTA/B7-H5/GI24, HVEM/TNFRSF14, BTLA, CD160, LAG3/CD223/Lymphocyte activation gene 3, CEACAM1/CD66a, indole amine, 2,3-dioxygenase/IDO, Galectin-/LGALS9, TIM-3/HAVCR2, 2B4/CD244, SIRP, alpha/CD172a, CD47, CD48/SLAMF2, TIGIT/VSTM3, A2AR, KIR, NOX2, SIGLEC7/CD328, SIGLEC9/CD329. In a further preferred embodiment, said at least one antibody capable of modulating an immune checkpoint protein is (ii) at least one antibody capable of antagonizing a co-inhibitory immune checkpoint protein, wherein said co-inhibitory immune checkpoint protein is selected from the group consisting of PD-1, PD-L1, PD-L2, CTLA-4, CD80/B7-1, CD86/B7-2, B7-H3/CD276, B7-H4/B7S1/7x, VISTA/B7-H5/GI24, HVEM/TNFRSF14, BTLA, CD160, LAG3/CD223/Lymphocyte activation gene 3, CEACAM1/CD66a, indole amine, Galectin-/LGALS9, TIM-3/HAVCR2, 2B4/CD244, SIRP, alpha/CD172a, CD47, CD48/SLAMF2, and TIGIT/VSTM3. In a further preferred embodiment, said at least one antibody capable of modulating an immune checkpoint protein is (ii) at least one antibody capable of antagonizing a co-inhibitory immune checkpoint protein, wherein said co-inhibitory immune checkpoint protein is selected from the group consisting of PD-1, PD-L1, PD-L2, CTLA-4, CD80/B7-1, CD86/B7-2, B7-H3/CD276, B7-H4/B7S1/7x, VISTA/B7-H5/GI24, LAG3/CD223/Lymphocyte activation gene 3, Galectin-/LGALS9, TIM-3/HAVCR2, and TIGIT/VSTM3. In a further preferred embodiment, said at least one antibody capable of modulating an immune checkpoint protein is (ii) at least one antibody capable of antagonizing a co-inhibitory immune checkpoint protein, wherein said co-inhibitory immune checkpoint protein is selected from the group consisting of PD-1, PD-L1, PD-L2, CTLA-4, CD80/B7-1, CD86/B7-2, B7-H3/CD276, B7-H4/B7S1/7x, VISTA/B7-H5/GI24, LAG3/CD223/Lymphocyte activation gene 3, Galectin-/LGALS9, TIM-3/HAVCR2, and TIGIT/VSTM3. In a further preferred embodiment, said at least one antibody capable of modulating an immune checkpoint protein is (ii) at least one antibody capable of antagonizing a co-inhibitory immune checkpoint protein, wherein said co-inhibitory immune checkpoint protein is selected from the group consisting of PD-1, PD-L1, PD-L2, CTLA-4, B7-H3/CD276, B7-H4/B7S1/7x, VISTA/B7-H5/GI24, HVEM/TNFRSF14, BTLA, CD160, LAG3/CD223/Lymphocyte activation gene 3, CEACAM1/CD66a, indole amine, 2,3-dioxygenase/IDO, Galectin-/LGALS9, TIM-3/HAVCR2, 2B4/CD244, SIRP, alpha/CD172a, CD47, CD48/SLAMF2, TIGIT/VSTM3, A2AR, KIR, NOX2, SIGLEC7/CD328, SIGLEC9/CD329. In a further preferred embodiment, said at least one antibody capable of modulating an immune checkpoint protein is (ii) at least one antibody capable of antagonizing a co-inhibitory immune checkpoint protein, wherein said co-inhibitory immune checkpoint protein is selected from the group consisting of PD-1, PD-L1, PD-L2, CTLA-4, B7-H3/CD276, B7-H4/B7S1/7x, VISTA/B7-H5/GI24, HVEM/TNFRSF14, BTLA, CD160, LAG3/CD223/Lymphocyte activation gene 3, CEACAM1/CD66a, indole amine, Galectin-/LGALS9, TIM-3/HAVCR2, 2B4/CD244, SIRP, alpha/CD172a, CD47, CD48/SLAMF2, and TIGIT/VSTM3. In a further preferred embodiment, said at least one antibody capable of modulating an immune checkpoint protein is (ii) at least one antibody capable of antagonizing a co-inhibitory immune checkpoint protein, wherein said co-inhibitory immune checkpoint protein is selected from the group consisting of PD-1, PD-L1, PD-L2, CTLA-4, B7-H3/CD276, B7-H4/B7S1/7x, VISTA/B7-H5/GI24, LAG3/CD223/Lymphocyte activation gene 3, Galectin-/LGALS9, TIM-3/HAVCR2, and TIGIT/VSTM3. In a further preferred embodiment, said at least one antibody capable of modulating an immune checkpoint protein is (ii) at least one antibody capable of antagonizing a co-inhibitory immune checkpoint protein, wherein said co-inhibitory immune checkpoint protein is selected from the group consisting of PD-1, PD-L1, PD-L2, CTLA-4, B7-H3/CD276, B7-H4/B7S1/7x, VISTA/B7-H5/GI24, LAG3/CD223/Lymphocyte activation gene 3, Galectin-/LGALS9, TIM-3/HAVCR2, and TIGIT/VSTM3. In a further preferred embodiment, said at least one antibody capable of modulating an immune checkpoint protein is (ii) at least one antibody capable of antagonizing a co-inhibitory immune checkpoint protein, wherein said co-inhibitory immune checkpoint protein is selected from the group consisting of PD-1, PD-L1, PD-L2, CTLA-4, TIGIT, LAG3, TIM3, B7-H3, B7-H4, VISTA, CCR4, and GITRL. In a further preferred embodiment, said at least one antibody capable of modulating an immune checkpoint protein is (ii) at least one antibody capable of antagonizing a co-inhibitory immune checkpoint protein, wherein said co-inhibitory immune checkpoint protein is selected from the group consisting of PD-1, PD-L1, PD-L2, CTLA-4, LAG3/CD223/Lymphocyte activation gene 3, TIM-3/HAVCR2, and TIGIT/VSTM3. In a further preferred embodiment, said at least one antibody capable of modulating an immune checkpoint protein is (ii) at least one antibody capable of antagonizing a co-inhibitory immune checkpoint protein, wherein said co-inhibitory immune checkpoint protein is selected from the group consisting of PD-1, PD-L1, and PD-L2.

In a further preferred embodiment of the composition or kit-of-parts of the invention, said immune checkpoint protein is selected from the group consisting of 4-1BB, 4-1BB ligand, PD-1, PD-L1, PD-L2 and CTLA-4. More preferably said immune checkpoint protein is selected from the group consisting of 4-1BB, 4-1BB ligand, PD-1, PD-L1, and PD-L2. In a further preferred embodiment of the composition or kit-of-parts of the invention, said immune checkpoint protein is selected from the group consisting of 4-1BB, PD-1, PD-L1, PD-L2 and CTLA-4. More preferably said immune checkpoint protein is selected from the group consisting of 4-1BB, PD-1, PD-L1, and PD-L2.

In a further preferred embodiment, said at least one antibody capable of modulating an immune checkpoint protein is (ii) at least one antibody capable of antagonizing a co-inhibitory immune checkpoint protein, wherein said antagonizing antibody is selected from the group consisting of anti-PD-1, anti-PD-L1, anti-anti-PD-L2, anti-CTLA-4, anti-LAG3/CD223/Lymphocyte activation gene 3, anti-TIM-3/HAVCR2, and anti-TIGIT/VSTM3. In a further preferred embodiment, said at least one antibody capable of modulating an immune checkpoint protein is (ii) at least one antibody capable of antagonizing a co-inhibitory immune checkpoint protein, wherein said antagonizing antibody is selected from the group consisting of anti-PD-1, anti-PD-L1, and anti-PD-L2.

In a preferred embodiment, said composition or kit-of-parts of the invention comprises more than one antibody capable of modulating an immune checkpoint protein. In a preferred embodiment, said composition or kit-of-parts of the invention comprises 1, 2 or 3 antibodies capable of modulating an immune checkpoint protein. In a preferred embodiment, said composition or kit-of-parts of the invention comprises 2 or 3 antibodies capable of modulating an immune checkpoint protein. In a preferred embodiment, said composition or kit-of-parts of the invention comprises 2 antibodies capable of modulating an immune checkpoint protein. In a preferred embodiment, said composition or kit-of-parts of the invention comprises 3 antibodies capable of modulating an immune checkpoint protein.

In a preferred embodiment, said composition or kit-of-parts of the invention further comprises a chemotherapeutic agent. In a preferred embodiment, said composition or kit-of-parts of the invention is combined with radiotherapy.

In a preferred embodiment, said at least one antibody capable of modulating an immune checkpoint protein included in the composition or kit-of-parts of the invention is a mixture of

(i) an antibody capable of modulating immune checkpoint protein CD27 and at least one antibody capable of modulating an immune checkpoint protein selected from the group consisting of PD-1, PD-L1, and PD-L2;
(ii) an antibody capable of modulating immune checkpoint protein CD40 and at least one antibody capable of modulating an immune checkpoint protein selected from the group consisting of PD-1, PD-L1, PD-L2, and CTLA-4;
(iii) an antibody capable of modulating immune checkpoint protein GITR and at least one antibody capable of modulating an immune checkpoint protein selected from the group consisting of PD-1, PD-L1, PD-L2, and CTLA-4;
(iv) an antibody capable of modulating immune checkpoint protein OX40 and at least one antibody capable of modulating an immune checkpoint protein selected from the group consisting of PD-1, PD-L1, PD-L2, 4-1BB, and CTLA-4;
(v) an antibody capable of modulating immune checkpoint protein 4-1BB and at least one antibody capable of modulating an immune checkpoint protein selected from the group consisting of PD-1, PD-L1, PD-L2, OX40, LAG-3 and CTLA-4; or
(vi) an antibody capable of modulating immune checkpoint protein ICOS and at least one antibody capable of modulating an immune checkpoint protein selected from the group consisting of PD-1, PD-L1, PD-L2 and CTLA-4.

In a preferred embodiment, said at least one antibody capable of modulating an immune checkpoint protein, included in the composition or kit-of-parts of the invention, is a mixture of (i) an antibody capable of modulating immune checkpoint protein CD27 and at least one antibody capable of modulating an immune checkpoint protein selected from the group consisting of PD-1, PD-L1, and PD-L2. In a preferred embodiment, said at least one antibody capable of modulating an immune checkpoint protein, included in the composition or kit-of-parts of the invention, is a mixture of (ii) an antibody capable of modulating immune checkpoint protein CD40 and at least one antibody capable of modulating an immune checkpoint protein selected from the group consisting of PD-1, PD-L1, PD-L2, and CTLA-4. In a preferred embodiment, said at least one antibody capable of modulating an immune checkpoint protein, included in the composition or kit-of-parts of the invention, is a mixture of (iii) an antibody capable of modulating immune checkpoint protein GITR and at least one antibody capable of modulating an immune checkpoint protein selected from the group consisting of PD-1, PD-L1, PD-L2 and CTLA-4. In a preferred embodiment, said at least one antibody capable of modulating an immune checkpoint protein, included in the composition or kit-of-parts of the invention, is a mixture of (iv) an antibody capable of modulating immune checkpoint protein OX40 and at least one antibody capable of modulating an immune checkpoint protein selected from the group consisting of PD-1, PD-L1, PD-L2, 4-1BB, and CTLA-4. In a preferred embodiment, said at least one antibody capable of modulating an immune checkpoint protein, included in the composition or kit-of-parts of the invention, is a mixture of (v) an antibody capable of modulating immune checkpoint protein 4-1BB and at least one antibody capable of modulating an immune checkpoint protein selected from the group consisting of PD-1, PD-L1, PD-L2, OX40, LAG-3 and CTLA-4. In a preferred embodiment, said at least one antibody capable of modulating an immune checkpoint protein, included in the composition or kit-of-parts of the invention, is a mixture of (vi) an antibody capable of modulating immune checkpoint protein ICOS and at least one antibody capable of modulating an immune checkpoint protein selected from the group consisting of PD-1, PD-L1, and PD-L2.

In a preferred embodiment, said at least one antibody capable of modulating an immune checkpoint protein, included in the composition or kit-of-parts of the invention, is a mixture of (i) anti-CD27 and at least one antibody capable of modulating an immune checkpoint protein, said antibody is selected from the group consisting of anti-PD-1, anti-PD-L1, and anti-PD-L2. In a preferred embodiment, said at least one antibody capable of modulating an immune checkpoint protein, included in the composition or kit-of-parts of the invention, is a mixture of (ii) anti-CD40 and at least one antibody capable of modulating an immune checkpoint protein, said antibody is selected from the group consisting of anti-PD-1, anti-PD-L1, anti-PD-L2, and anti-CTLA-4. In a preferred embodiment, said at least one antibody capable of modulating an immune checkpoint protein, included in the composition or kit-of-parts of the invention, is a mixture of (iii) anti-GITR and at least one antibody capable of modulating an immune checkpoint protein, said antibody is selected from the group consisting of anti-PD-1, anti-PD-L1, anti-PD-L2 and anti-CTLA-4. In a preferred embodiment, said at least one antibody capable of modulating an immune checkpoint protein, included in the composition or kit-of-parts of the invention, is a mixture of (iv) anti-OX40 and at least one antibody capable of modulating an immune checkpoint protein, said antibody is selected from the group consisting of anti-PD-1, anti-PD-L1, anti-PD-L2, anti-4-1BB, and anti-CTLA-4. In a preferred embodiment, said at least one antibody capable of modulating an immune checkpoint protein, included in the composition or kit-of-parts of the invention, is a mixture of (v) anti-4-1BB and at least one antibody capable of modulating an immune checkpoint protein, said antibody is selected from the group consisting of anti-PD-1, anti-PD-L1, anti-PD-L2, anti-OX40, anti-LAG-3 and anti-CTLA-4. In a preferred embodiment, said at least one antibody capable of modulating an immune checkpoint protein, included in the composition or kit-of-parts of the invention, is a mixture of (vi) anti-ICOS and at least one antibody capable of modulating an immune checkpoint protein, said antibody is selected from the group consisting of anti-PD-1, anti-PD-L1, and anti-PD-L2.

In a preferred embodiment, said polyplex of the invention comprises a double stranded RNA (dsRNA) and a polymeric conjugate, wherein said polymeric conjugate comprises a polyethyleneimine (PEI), one or more polyethylene glycol (PEG) moieties, one or more linkers and one or more targeting moieties; wherein said PEI is covalently bound to one or more PEG moieties, and each of said one or more PEG moieties is linked to one of said one or more targeting moieties via one of said one or more linkers, and wherein each of said one or more targeting moieties is capable of binding to a cancer antigen.

In a preferred embodiment, said polyplex of the invention comprises a double stranded RNA (dsRNA) and a polymeric conjugate, wherein said polymeric conjugate consists of a polyethyleneimine (PEI), one or more polyethylene glycol (PEG) moieties, one or more linkers and one or more targeting moieties; wherein said PEI is covalently bound to one or more PEG moieties, and each of said one or more PEG moieties is linked to one of said one or more targeting moieties via one of said one or more linkers, and wherein each of said one or more targeting moieties is capable of binding to a cancer antigen.

In a preferred embodiment, said polyplex of the invention consists of a double stranded RNA (dsRNA) and a polymeric conjugate, wherein said polymeric conjugate consists of a polyethyleneimine (PEI), one or more polyethylene glycol (PEG) moieties, one or more linkers and one or more targeting moieties; wherein said PEI is covalently bound to one or more PEG moieties, and each of said one or more PEG moieties is linked to one of said one or more targeting moieties via one of said one or more linkers, and wherein each of said one or more targeting moieties is capable of binding to a cancer antigen.

The polyplex of the invention comprises a polyplex comprising a double stranded RNA (dsRNA). The term “dsRNA” typically and preferably refers to double stranded ribonucleotide polymers of any length in which one or more ribonucleotides can be chemical analogues or modified derivatives of a corresponding naturally-occurring ribonucleotide. The term “dsRNA” typically and preferably also includes mismatched dsRNA.

In a preferred embodiment, said dsRNA is polyinosinic-polycytidylic acid double stranded RNA (polyIC or pIC). PolyIC is a double-stranded RNA with one strand being a polymer of inosinic acid, the other a polymer of cytidylic acid.

The polyIC of the polyplex for use according to the invention may be composed of dsRNA, wherein each strand consists of at least 22, preferably at least 45 ribonucleotides. In a certain embodiment, each strand consists of 20 to 8000 ribonucleotides. In a certain embodiment, each strand consists of 20 to 4000 ribonucleotides. In a more preferred embodiment each strand consists of 20 to 300 ribonucleotides.

The term “molecular weight” as used herein and, in particular, when referring to polymers such as polyIC, PEI and PEG, refers to average molecular weight, preferably to weight average molecular weight.

PolyIC is bound to the polymeric conjugate via non-covalent or covalent bonds, wherein non-covalent binding is preferred. In a preferred embodiment, said polyIC is non-covalently bound to PEI, preferably by ionic bonds.

The polyplex according to the invention comprises a polymeric conjugate, wherein said polymeric conjugate comprises polyethyleneimine (PEI) which is a polycation with the capacity to condense and associate non-covalently with nucleic acid molecules due to the polyanionic nature of the latter.

In a preferred embodiment, said polyethyleneimine (PEI) is linear polyethyleneimine (LPEI). In a preferred embodiment, LPEI includes a hydroxyl group located at one or either end of LPEI. Preferably, said hydroxyl group is instead of the terminal —NH₂ group of LPEI.

In a preferred embodiment of the invention, PEI or preferably LPEI has a molecular weight from about 10-30 kDa. In a preferred embodiment of the invention, PEI or preferably LPEI has a molecular weight from about 15-25 kDa (PEI₁₅₋₂₅ₖ//LPEI₁₅₋₂₅ₖ). In a further more preferred embodiment, PEI or preferably LPEI has a molecular weight of about 22 kDa (LPEI₂₂ₖ). In an again further preferred embodiment, PEI or preferably LPEI has a molecular weight of about 20 kDa. The expression “LPEI has a molecular weight of about 22 kDa” is abbreviated and used synonymously herein with “LPEI₂₂ₖ”. The expression “PEI has a molecular weight of about 22 kDa” is abbreviated and used synonymously herein with “PEI₂₂ₖ”).

In a preferred embodiment, said PEI or preferably LPEI has a low dispersity. Preferably, the polydispersity index PDI of PEI or LPEI is about 1.

In a preferred embodiment, said one or more PEG moieties each independently forms —NH—CO— bond with said PEI or preferably LPEI.

The polyplex for use according to the invention includes one or more polyethylene glycol (PEG) moieties. PEG moieties according to the invention are also known as polyethylene oxide (PEO) or polyoxyethylene (POE) moieties, depending on its molecular weight. As used herein the term “polyethylene glycol moiety” (PEG moiety) typically and preferably refers to a PEG moiety comprising two functionalities located on either end of polyethylene glycol (PEG). Said functionalities are capable of reacting with either said PEI or preferably LPEI or said targeting moiety.

In a preferred embodiment, said PEG moiety is linear or branched. In another preferred embodiment, said PEG moiety is branched. In a further preferred embodiment, said PEG moiety is linear.

In one embodiment of the invention, each of said at least one PEG moiety has a molecular weight from 1 kD or more. In another embodiment, each of said at least one PEG moiety has a molecular weight from about 0.3-8 kDa, preferably about 0.5-5 Da, more preferably, 1-3 kDa (PEG₁₋₃ₖ), most preferably 2 kDa (PEG₂ₖ). As indicated, said molecular weight corresponds to average molecular weight, Thus, the preferred and used PEG₂ₖ refers to a mixture of polyethylene glycols having an average value n of between 30 and 60 (with some even smaller and some larger) of —(CH₂CH₂O)ₙ units and a molecular weight range from about 1300 to 2600 grams/mole.

In a preferred embodiment, said PEI has a molecular weight of about 10-30 kDa, and said at least one PEG moiety has a molecular weight of about 0.3-8 kDa. In a more preferred embodiment, PEI has a molecular weight of about 22 kDa (PEI₂₂ₖ), and said at least one PEG moiety has a molecular weight of about 2 kDa (PEG₂ₖ). In a more preferred embodiment, PEI is LPEI having a molecular weight of about 20 kDa (LPEI₂₀ₖ), and said at least one PEG moiety has a molecular weight of about 2 kDa (PEG₂ₖ).

In a preferred embodiment, PEI is covalently linked to one to five PEG moieties, preferably PEI is covalently linked to one to three PEG moieties. In a preferred embodiment, LPEI is covalently linked to one to five PEG moieties, wherein preferably LPEI is covalently linked to one to three PEG moieties. In a preferred embodiment, PEI₁₅₋₂₅ₖ, preferably PEI₂₂ₖ, is covalently linked to one to five PEG₁₋₃ₖ, preferably PEG₂ₖ, moieties, wherein preferably PEI₁₅₋₂₅ₖ, further preferably PEI₂₂ₖ, is covalently linked to one to three PEG₁₋₃ₖ, preferably PEG₂ₖ, moieties. In a preferred embodiment, LPEI₁₅₋₂₅ₖ, preferably LPEI₂₂ₖ, is covalently linked to one to five PEG₁₋₃ₖ, preferably PEG₂ₖ, moieties, wherein preferably PEI₁₅₋₂₅ₖ, further preferably PEI₂₂ₖ, is covalently linked to one to three PEG₁₋₃ₖ, preferably PEG₂ₖ, moieties. In a preferred embodiment, PEI₁₅₋₂₅ₖ, preferably PEI₂₂ₖ, is covalently linked to one PEG₁₋₃ₖ, preferably PEG₂ₖ moiety or two or three PEG₁₋₃ₖ, preferably PEG₂ₖ moieties. In another preferred embodiment, PEI₁₅₋₂₅ₖ, preferably PEI₂₂ₖ is covalently linked to one PEG₁₋₃ₖ, preferably PEG₂ₖ. In a further preferred embodiment, PEI₁₅₋₂₅ₖ, preferably PEI₂₂ₖ, is covalently linked to two PEG₁₋₃ₖ, preferably PEG₂ₖ, moieties. In a further preferred embodiment, PEI₁₅₋₂₅ₖ, preferably PEI₂₂ₖ is covalently linked to three PEG₁₋₃ₖ, preferably PEG₂ₖ, moieties. In a preferred embodiment, LPEI₁₅₋₂₅ₖ, preferably LPEI₂₂ₖ, is covalently linked to one PEG₁₋₃ₖ, preferably PEG₂ₖ, moiety or two or three PEG₁₋₃ₖ, preferably PEG₂ₖ, moieties. In another preferred embodiment, LPEI₁₅₋₂₅ₖ, preferably LPEI₂₂ₖ, is covalently linked to one PEG₁₋₃ₖ, preferably PEG₂ₖ. In a further preferred embodiment, LPEI₁₅₋₂₅ₖ, preferably LPEI₂₂ₖ, is covalently linked to two PEG₁₋₃ₖ, preferably PEG₂ₖ, moieties. In a further preferred embodiment, LPEI₁₅₋₂₅ₖ, preferably LPEI₂₂ₖ, is covalently linked to three PEG₁₋₃ₖ, preferably PEG₂ₖ, moieties. In another preferred embodiment, said PEI of the polyplex is covalently bound to one, two or three PEG moieties. Preferably, said PEI of the polyplex is covalently bound to one or three PEG moieties. In another more preferred embodiment, said PEI of the polyplex is LPEI covalently bound to one, two or three PEG moieties. More preferably, said PEI of the polyplex is LPEI covalently bound to one or three PEG moieties.

As used herein, the term “PEI [ . . . ] covalently linked to one PEG moiety” (used interchangeably herein with “PEI-PEG 1:1”) or “LPEI [ . . . ] covalently linked to one PEG moiety” (used interchangeably herein with “LPEI-PEG 1:1”) refers to the molar ratio of PEI to PEG or LPEI to PEG, wherein PEI-PEG 1:1 or LPEI-PEG 1:1 typically and preferably means that approximately one mole PEG per one mole PEI or LPEI is included in the polymeric conjugate. As used herein, the term “PEI [ . . . ] covalently linked to three PEG moieties” (used interchangeably herein with “PEI-PEG 1:3”) or the term “LPEI [ . . . ] covalently linked to three PEG moieties” (used interchangeably herein with “LPEI-PEG 1:3”) typically and preferably means that approximately three moles PEG per one mole PEI or LPEI are included in the polymeric conjugate. The values are preferably determined by ¹H-NMR analysis. The relative integral values of the hydrogen atoms on PEG (—CH₂—CH₂—O—) and the integral values of the hydrogen atoms on PEI or LPEI (—CH₂—CH₂—NH—) are preferably used for determining the values via ¹H-NMR. The term “approximately” herein refers preferably to a deviation of about 0%-10%, more preferably about 0%-5%, again more preferably about 0%-2%.

In a preferred embodiment, said dsRNA is polyIC and said PEI is covalently linked to one to three PEG moieties. In a preferred embodiment, said dsRNA is polyIC and said PEI is covalently linked to one, two or three PEG moieties. In a more preferred embodiment, said dsRNA is polyIC and said PEI is LPEI covalently linked to one to three PEG moieties. In a more preferred embodiment, said dsRNA is polyIC and said PEI is LPEI covalently linked to one, two or three PEG moieties.

In a preferred embodiment, said dsRNA is polyIC and said PEI is covalently linked to one, two or three PEG moieties. In a more preferred embodiment, said dsRNA is polyIC and said PEI is LPEI covalently linked to one, two or three PEG moieties. In a more preferred embodiment, said dsRNA is polyIC and said PEI is LPEI₂₂ₖ covalently linked to one, two or three PEG moieties. In a more preferred embodiment, said dsRNA is polyIC and said PEI is LPEI covalently linked to one, two or three PEG_{0.3-8k}, preferably PEG₂ₖ moieties. In a more preferred embodiment, said dsRNA is polyIC and said PEI is LPEI₂₂ₖ covalently linked to one, two or three PEG moieties. In a more preferred embodiment, said dsRNA is polyIC and said PEI is LPEI₂₂ₖ covalently linked to one, two or three PEG_{0.3-8k}, preferably PEG₂ₖ moieties.

In a preferred embodiment, said dsRNA is polyIC and said PEI is covalently linked to one PEG moiety (PEI-PEG 1:1). In a more preferred embodiment, said dsRNA is polyIC and said PEI is LPEI covalently linked to one PEG moiety (LPEI-PEG 1:1). In a more preferred embodiment, said dsRNA is polyIC and said PEI is LPEI₂₂ₖ covalently linked to one PEG moiety. In a more preferred embodiment, said dsRNA is polyIC and said PEI is LPEI covalently linked to one PEG_{0.3-8k}, preferably PEG₂ₖ moiety (LPEI-PEG 1:1). In a more preferred embodiment, said dsRNA is polyIC and said PEI is LPEI₂₂ₖ covalently linked to one PEG moiety. In a more preferred embodiment, said dsRNA is polyIC and said PEI is LPEI₂₂ₖ covalently linked to one PEG_{0.3-8k}, preferably PEG₂ₖ moiety (LPEI-PEG 1:1).

In a preferred embodiment, said dsRNA is polyIC and said PEI is covalently linked to three PEG moieties. In a more preferred embodiment, said dsRNA is polyIC and said PEI is LPEI covalently linked to three PEG moieties. In a more preferred embodiment, said dsRNA is polyIC and said PEI is LPEI₂₂ₖ covalently linked to three PEG moieties. In a more preferred embodiment, said dsRNA is polyIC and said PEI is LPEI covalently linked to three PEG_{0.3-8k}, preferably PEG₂ₖ moieties. In a more preferred embodiment, said dsRNA is polyIC and said PEI is LPEI₂₂ₖ covalently linked to three PEG moieties. In a more preferred embodiment, said dsRNA is polyIC and said PEI is LPEI₂₂ₖ covalently linked to three PEG_{0.3-8k}, preferably PEG₂ₖ moieties.

In a preferred embodiment, said dsRNA of the polyplex is polyIC and said PEI of the polymeric conjugate of the polyplex for use according to the invention is LPEI₂₂ₖ. In a preferred embodiment, said dsRNA of the polyplex is polyIC and said PEI of the polymeric conjugate of the polyplex for use according to the invention is LPEI₂₂ₖ covalently linked to one, two or three PEG moieties.

In a preferred embodiment, said dsRNA is polyIC and said one or more PEG moieties of the polymeric conjugate are PEG_{0.3-8k}, preferably PEG₂ₖ. In a preferred embodiment, said dsRNA is polyIC and said LPEI is covalently linked to one, two or three PEG_{0.3-8k}, preferably PEG₂ₖ moieties. In a preferred embodiment, said dsRNA is polyIC and said LPEI is covalently linked to one PEG_{0.3-8k}, preferably PEG₂ₖ moieties (LPEI-PEG 1:1). In a preferred embodiment, said dsRNA is polyIC and said LPEI is covalently linked to PEG_{0.3-8k}, preferably PEG₂ₖ moieties (LPEI-PEG 1:3).

In a preferred embodiment, said dsRNA of the polyplex is polyIC, said LPEI of the polymeric conjugate is LPEI₂₂ₖ and said one or more PEG moieties are PEG_{0.3-8k}, preferably PEG₂ₖ moiety. In a preferred embodiment, said dsRNA of the polyplex is polyIC and said LPEI is LPEI₂₂ₖ and is covalently linked to one PEG_{0.3-8k}, preferably PEG₂ₖ moiety (LPEI-PEG 1:1) or three PEG_{0.3-8k}, preferably PEG₂ₖ moieties (LPEI-PEG 1:3).

The term “cancer antigen” as used herein (synonymously used herein with tumor antigen or tumor marker) refers to an antigenic substance produced in tumor cells or presented on tumor cells which triggers an immune response in the host. In certain embodiments, said cancer antigen is selected from the group consisting of an epidermal growth factor receptor (EGFR), human epidermal growth factor receptor 2 (HER2), prostate surface membrane antigen (PSMA), an insulin-like growth factor 1 receptor (IGF1R), a vascular endothelial growth factor receptor (VEGFR), a platelet-derived growth factor receptor (PDGFR), and a fibroblast growth factor receptor (FGFR). In a further preferred embodiment, said cancer antigen is selected from the group consisting of an epidermal growth factor receptor (EGFR), human epidermal growth factor receptor 2 (HER2), and prostate surface membrane antigen (PSMA). In another preferred embodiment, said cancer antigen is EGFR. In another preferred embodiment, said cancer antigen is PSMA. In another preferred embodiment, said cancer antigen is a HER2. In another preferred embodiment, said cancer antigen is EGFR or HER2. In another preferred embodiment, said cancer antigen is EGFR or PSMA.

In another preferred embodiment, said dsRNA is polyIC and said PEI is LPEI covalently linked to one to three PEG moieties and said cancer antigen is EGFR or PSMA. In another preferred embodiment, said dsRNA is polyIC and said PEI is LPEI covalently linked to one to three PEG moieties and said cancer antigen is EGFR. In another preferred embodiment, said dsRNA is polyIC and said PEI is LPEI covalently linked to one to three PEG moieties and said cancer antigen is PSMA. In another preferred embodiment, said dsRNA is polyIC and said PEI is LPEI covalently linked to one to three PEG moieties and said cancer antigen is HER2.

In another preferred embodiment, said dsRNA is polyIC and said PEI is LPEI covalently linked to one to three PEG moieties and said targeting moiety is EGF, preferably human EGF, HER2 affibody, HER2 antibody or DUPA. In another preferred embodiment, said dsRNA is polyIC and said PEI is LPEI covalently linked to one to three PEG moieties and said targeting moiety is EGF, preferably human EGF. In another preferred embodiment, said dsRNA is polyIC and said PEI is LPEI covalently linked to one to three PEG moieties and said targeting moiety is a HER2 affibody or HER2 antibody.

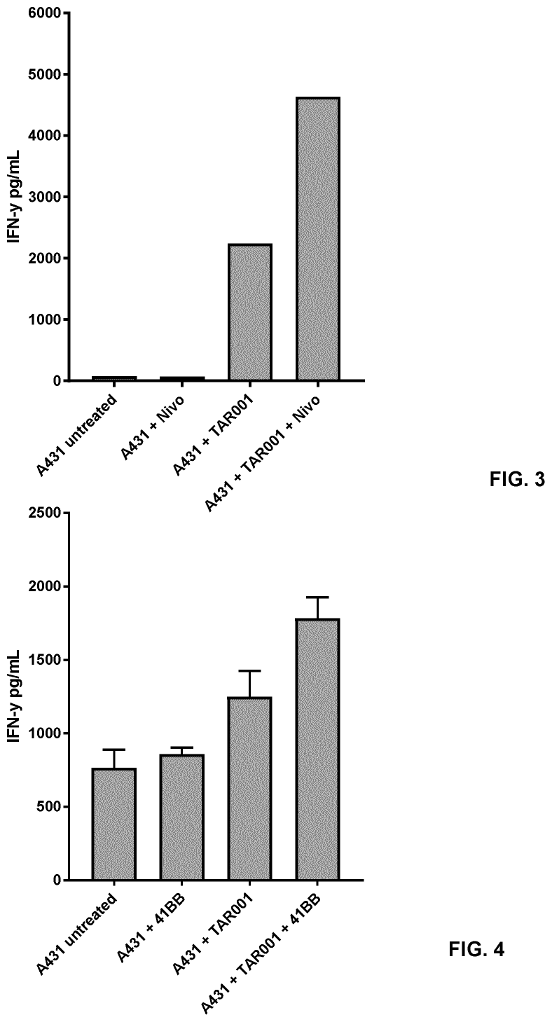

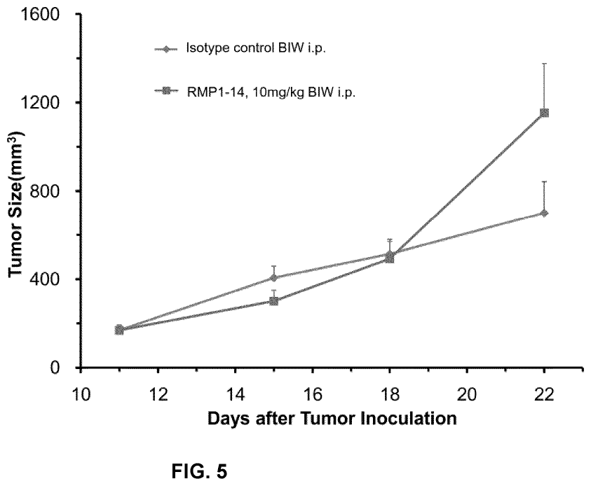

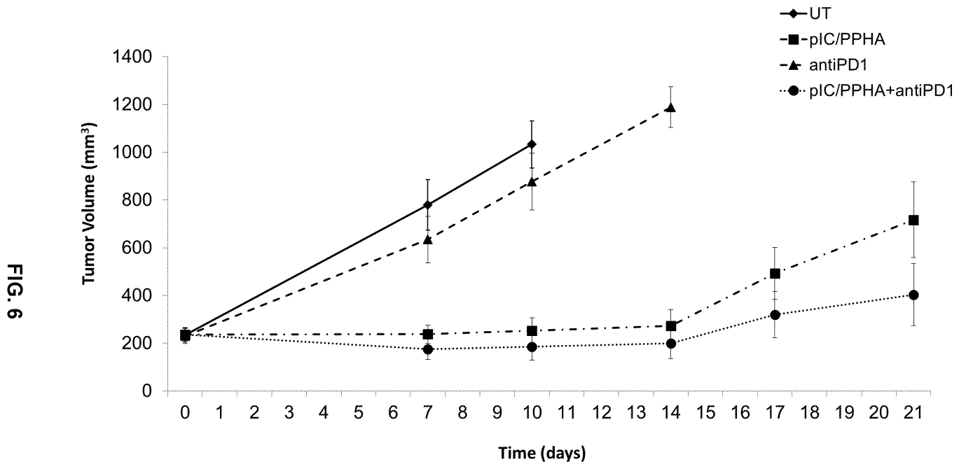

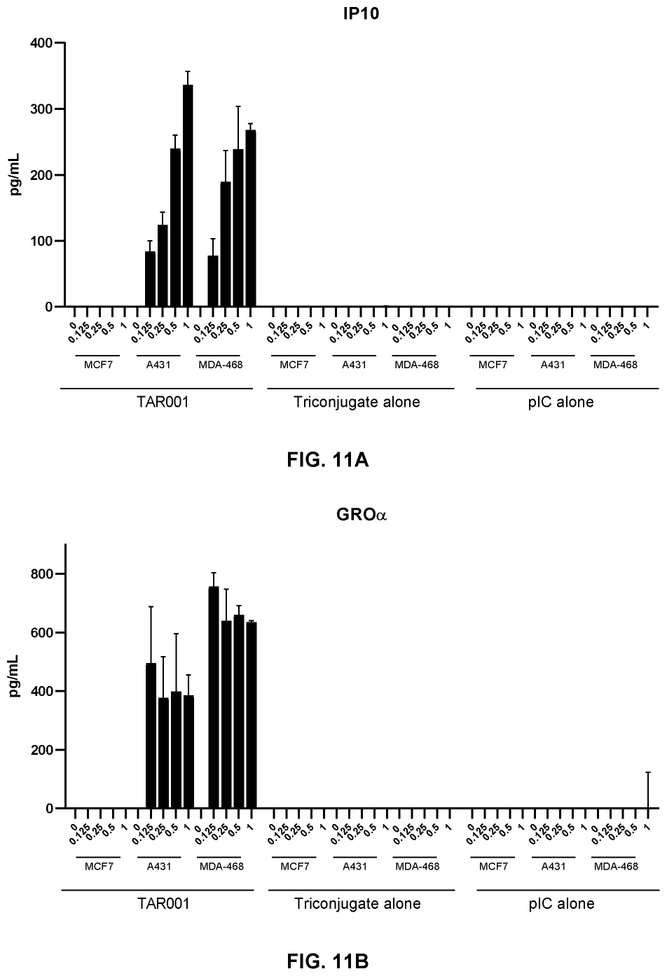

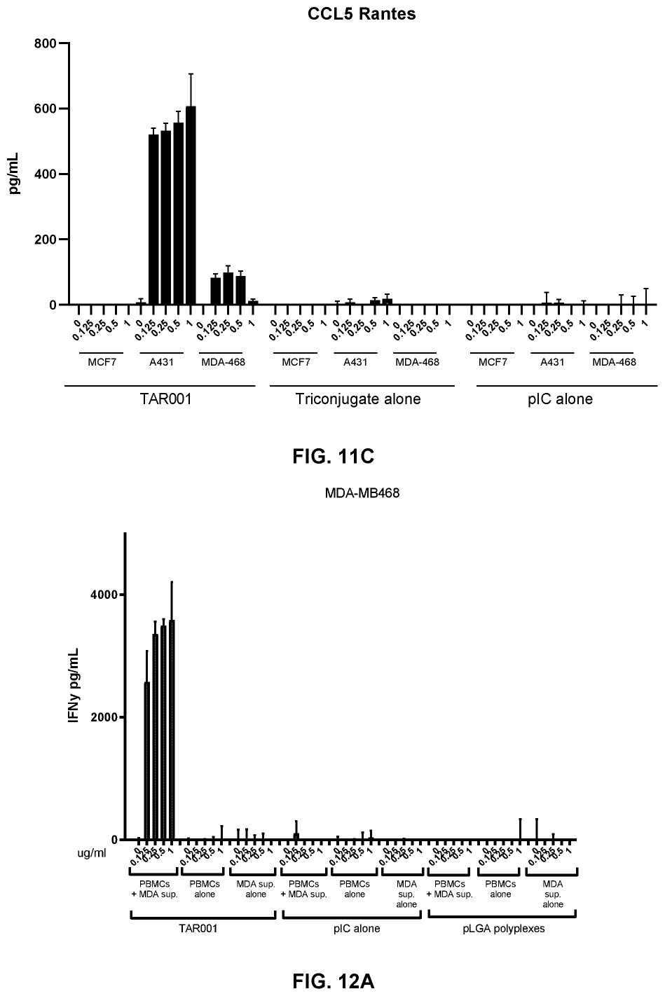

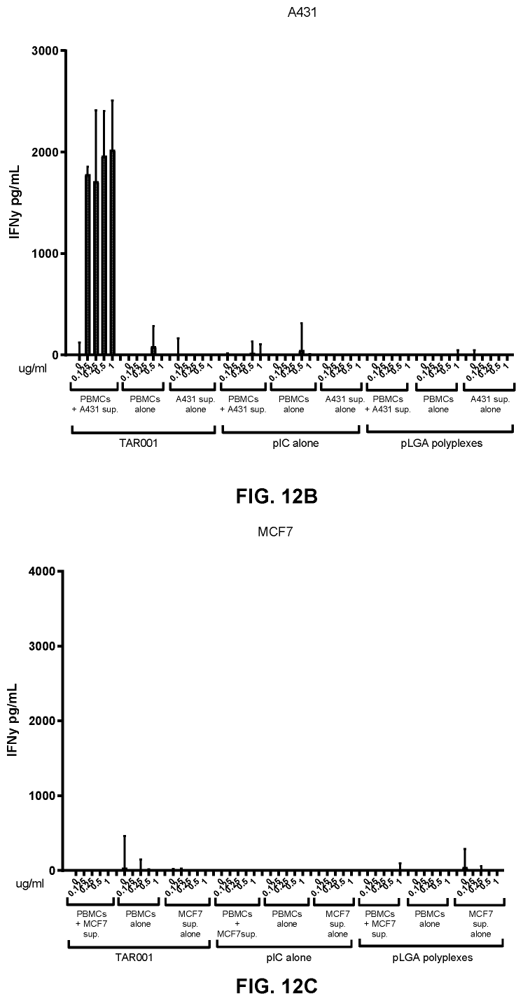

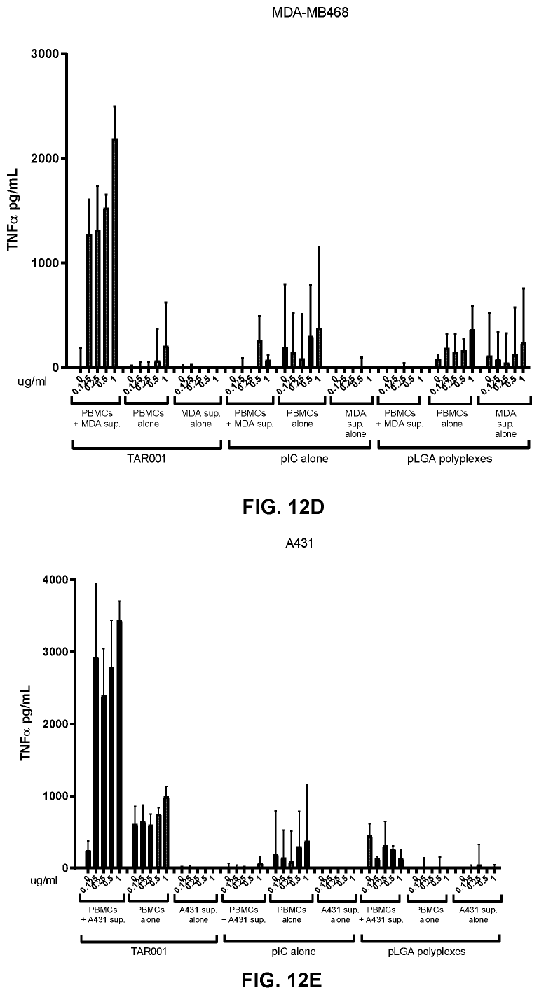

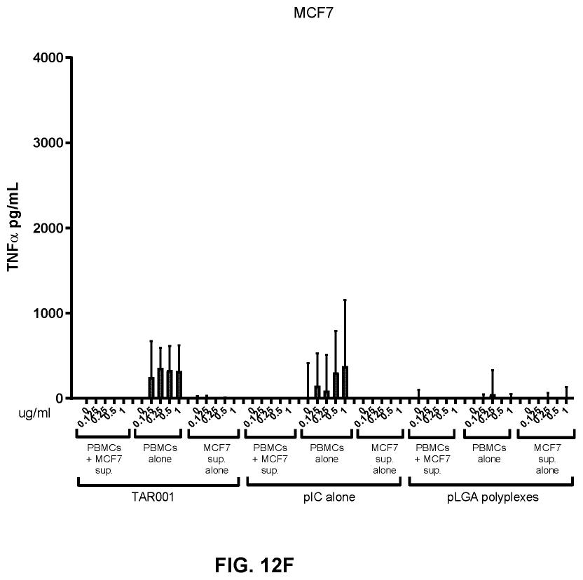

PEI-PEG-EGF (Epidermal Growth Factor)/polyIC polyplex treatment of high EGFR (Epidermal Growth Factor Receptor)-expressing cells induced the secretion of the pro-inflammatory cytokines (IP-10, GRO-α and CCL5), which was not observed in low EGFR-expressing cells (see herein FIG. 11, Example 8). Moreover, increased secretion of cytokines (IFN-γ and TNFα) was observed in PBMCs incubated with supernatant from PEG-EGF/polyIC polyplex-treated cells with high EGFR-expression which was not observed for media from low EGFR-expressing cells (see herein FIG. 12, Example 9). Combination of PEI-PEG-EGF/polyIC with Nivolumab or anti-41BB antibodies led to a further increase in cytokine IFN-y release in PBMCs as compared to treatment with PEI-PEG-EGF/polyIC, Nivolumab or anti-41BB antibodies alone (see herein FIGS. 3, 4 and 10, Examples 2 and 7). Treatment of mice bearing RENCA EGFR tumors with PEI-PEG-EGF/polyIC in combination with anti-PD-1 showed an increase in survival as compared to anti-PD-1 or PEI-PEG-EGF/polyIC evidencing a synergistic effect achieved with the inventive composition and combination treatment, respectively, as shown herein in FIG. 5 and Example 3.

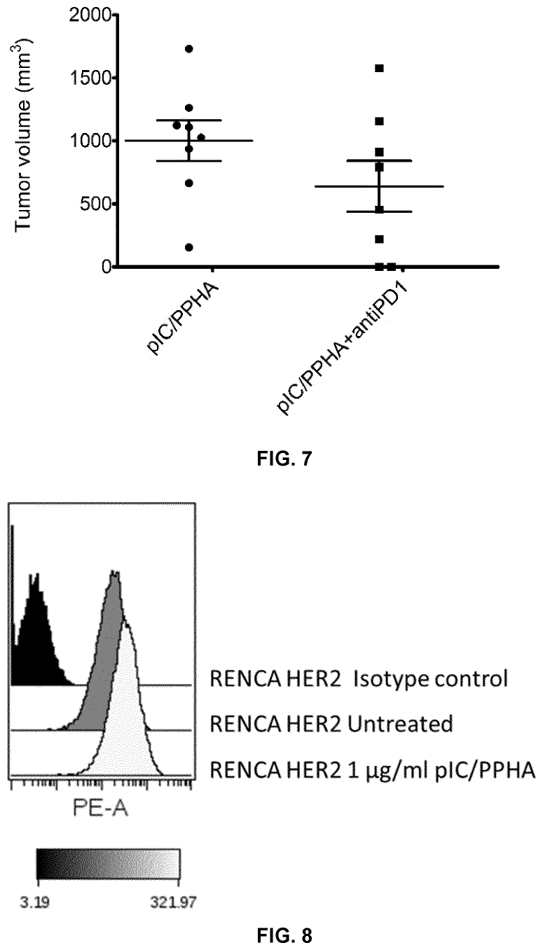

Analogously polyIC/PEI-PEG conjugated to a HER2 affibody (pIC/PPHA) treatment induced the expression of anti-proliferative (e.g., IFN-γ, IFN-α) and immunostimulatory cytokines (e.g., IP-10, GRO-alpha, and RANTES), triggering immune cell recruitment and activation and leading to extensive cancer cell killing in vitro (Zigler et al., Cancer Immunol Res; 4(8) August 2016). As described in Example 5 and as further shown in FIGS. 6-9 herein, the treatment of RENCA HER2 tumors with a combination of HER2 targeted polyIC polyplex (polyIC/PEI-PEG-HER2 affibody) plus anti-PD-1 in accordance with the present invention generated complete regression of tumor growth and complete protection from tumor re-challenge.

It is hereby of particular note that the vector polyIC/PEI-PEG-DUPA (pIC/PPD) comprising PEI-PEG tethered to the PSMA ligand DUPA (2-[3-(1, 3-dicarboxy propyl) ureido] pentanedioic acid) selectively delivered polyIC into PSMA-overexpressing prostate cancer cells inducing apoptosis, cytokine secretion, and the recruitment of human peripheral blood mononuclear cells (PBMCs). In particular, pIC/PPD led to the production of IFN-β, IP-10, and RANTES, to chemotaxis, and to PBMC activation which was evident from strong expression of IL-2 and led to the secretion of high levels of TNF-α and IFN-γ (Langut et al., PNAS, Dec. 26, 2017, vol. 114, no. 52).

Therefore, the above clearly confirms and supports that the inventive compositions and kit-of-parts comprising a polyplex and at least one antibody capable of modulating an immune checkpoint protein are able to lead to increased activation of the immune system and more potent antitumor activity. This is in particular the case for the preferred inventive compositions comprising polyplexes, wherein said cancer antigen is EGFR, HER2 or PSMA and/or said one or more targeting moieties are selected from EGF, a HER2 affibody, a HER2 antibody and DUPA, and wherein said antibody is capable of modulating an immune checkpoint protein, preferably an anti-4-1BB, anti-PD-1, anti-PD-L1 or anti-PD-L2 antibody.

In another preferred embodiment, said dsRNA is polyIC and said PEI is LPEI₁₅₋₂₅ₖ, preferably LPEI₂₂ₖ, covalently linked to one to three PEG moieties and said targeting moiety is EGF, preferably human EGF, HER2 affibody, HER2 antibody or DUPA. In another preferred embodiment, said dsRNA is polyIC and said PEI is LPEI₁₅₋₂₅ₖ, preferably LPEI₂₂ₖ, covalently linked to one to three PEG moieties and said targeting moiety is EGF, preferably human EGF. In another preferred embodiment, said dsRNA is polyIC and said PEI is LPEI₁₅₋₂₅ₖ, preferably LPEI₂₂ₖ, covalently linked to one to three PEG moieties and said targeting moiety is a HER2 affibody or HER2 antibody.

In another preferred embodiment, said dsRNA is polyIC and said PEI is LPEI covalently linked to one, two or three PEG_{0.3-8k}, preferably PEG₁₋₃ₖ, further preferably PEG₂ₖ moieties and said targeting moiety is EGF, preferably human EGF, HER2 affibody, HER2 antibody or DUPA. In another preferred embodiment, said dsRNA is polyIC and said PEI is LPEI covalently linked to one, two or three PEG_{0.3-8k}, preferably PEG₁₋₃ₖ, further preferably PEG₂ₖ moieties and said targeting moiety is EGF, preferably human EGF. In another preferred embodiment, said dsRNA is polyIC and said PEI is LPEI covalently linked to one to three PEG_{0.3-8k}, preferably PEG₁₋₃ₖ, further preferably PEG₂ₖ moieties and said targeting moiety is a HER2 affibody or HER2 antibody.

In another preferred embodiment, said dsRNA is polyIC and said PEI is LPEI₂₂ₖ covalently linked to one, two or three PEG_{0.3-8k}, preferably PEG₁₋₃ₖ, further preferably PEG₂ₖ moieties and said targeting moiety is EGF, preferably human EGF, HER2 affibody, HER2 antibody or DUPA. In another preferred embodiment, said dsRNA is polyIC and said PEI is LPEI₁₅₋₂₅ₖ, preferably LPEI₂₂ₖ, covalently linked to one, two or three PEG_{0.3-8k}, preferably PEG₁₋₃ₖ, further preferably PEG₂ₖ moieties and said targeting moiety is EGF, preferably human EGF. In another preferred embodiment, said dsRNA is polyIC and said PEI is LPEI₁₅₋₂₅ₖ, preferably LPEI₂₂ₖ, covalently linked to one to three PEG_{0.3-8k}, preferably PEG₁₋₃ₖ, further preferably PEG₂ₖ moieties and said targeting moiety is a HER2 affibody or HER2 antibody.

In another preferred embodiment, said dsRNA is polyIC, said PEI is LPEI covalently linked to one, two or three PEG moieties, said targeting moiety is EGF, preferably human EGF, and said immune checkpoint protein is selected from the group consisting of PD-1, PD-L1, PD-L2, CTLA-4, B7-1, B7-2, 4-1BB, 4-1BB ligand (4-1BBL), TIGIT, LAG3, TIM3, B7-H3, B7-H4, VISTA, CCR4, GITR ligand, GITR, OX40, OX-40L, ICOS, ICOS ligand CD40 and CD40 ligand. In another preferred embodiment, said dsRNA is polyIC, said PEI is LPEI covalently linked to one, two or three PEG moieties, said targeting moiety is EGF, preferably human EGF, and said immune checkpoint protein is selected from the group consisting of PD-1, PD-L1, PD-L2, CTLA-4, B7-1, B7-2, 4-1BB, 4-1BB ligand, TIGIT, LAG3, TIM-3, GITR, GITR ligand, CD40, CD40 ligand, OX40, OX40 ligand, ICOS ligand and ICOS.

In another preferred embodiment, said dsRNA is polyIC, said PEI is LPEI covalently linked to one, two or three PEG moieties, said targeting moiety is EGF, preferably human EGF, and said immune checkpoint protein is selected from the group consisting of PD-1, PD-L1, PD-L2, CTLA-4, 4-1BB, 4-1BB ligand (4-1BBL), TIGIT, LAG3, TIM3, B7-H3, B7-H4, VISTA, CCR4, GITR ligand, GITR, OX40, OX-40L, ICOS, ICOS ligand CD40 and CD40 ligand. In another preferred embodiment, said dsRNA is polyIC, said PEI is LPEI covalently linked to one, two or three PEG moieties, said targeting moiety is EGF, preferably human EGF, and said immune checkpoint protein is selected from the group consisting of PD-1, PD-L1, PD-L2, CTLA-4, 4-1BB, 4-1BB ligand, TIGIT, LAG3, TIM-3, GITR, GITR ligand, CD40, CD40 ligand, OX40, OX40 ligand, ICOS ligand and ICOS. In another preferred embodiment, said dsRNA is polyIC, said PEI is LPEI covalently linked to one, two or three PEG moieties, said targeting moiety is EGF, preferably human EGF, and said immune checkpoint protein is selected from the group consisting of PD-1, PD-L1, PD-L2, CTLA-4, B7-1, B7-2, 4-1BB, TIGIT, LAG3, TIM3, B7-H3, B7-H4, VISTA, CCR4, GITR, OX40, ICOS, and CD40. In another preferred embodiment, said dsRNA is polyIC, said PEI is LPEI covalently linked to one, two or three PEG moieties, said targeting moiety is EGF, preferably human EGF, and said immune checkpoint protein is selected from the group consisting of PD-1, PD-L1, PD-L2, CTLA-4, B7-1, B7-2, 4-1BB, TIGIT, LAG3, TIM-3, GITR, CD40, OX40, and ICOS.

In another preferred embodiment, said dsRNA is polyIC, said targeting moiety is EGF, preferably human EGF, and said immune checkpoint protein is selected from the group consisting of PD-1, PD-L1, PD-L2, 4-1BB, CTLA-4, GITR, CD40, OX40, and ICOS. In another preferred embodiment, said dsRNA is polyIC, said targeting moiety is EGF, preferably human EGF, and said immune checkpoint protein is selected from the group consisting of PD-1, PD-L1, PD-L2, 4-1BB, GITR, CD40, OX40, and ICOS. In another preferred embodiment, said dsRNA is polyIC, said targeting moiety is EGF, preferably human EGF, and said immune checkpoint protein is selected from the group consisting of PD-1, PD-L1, PD-L2, 4-1BB, GITR, CD40, and OX40. In another preferred embodiment, said dsRNA is polyIC, said targeting moiety is EGF, preferably human EGF, and said immune checkpoint protein is selected from the group consisting of PD-1, PD-L1, PD-L2, 4-1BB, CD40 and OX40. In another preferred embodiment, said dsRNA is polyIC, said targeting moiety is EGF, preferably human EGF, and said immune checkpoint protein is selected from the group consisting of PD-1, PD-L1, PD-L2, 4-1BB and OX40. In another preferred embodiment, said dsRNA is polyIC, said targeting moiety is EGF, preferably human EGF, and said immune checkpoint protein is selected from the group consisting of PD-1, PD-L1, PD-L2, and 4-1BB. In another preferred embodiment, said dsRNA is polyIC, said targeting moiety is EGF, preferably human EGF, and said immune checkpoint protein is selected from the group consisting of PD-1 and 4-1BB.

In another preferred embodiment, said dsRNA is polyIC, said PEI is LPEI covalently linked to one, two or three PEG moieties, said targeting moiety is EGF, preferably human EGF, and said immune checkpoint protein is selected from the group consisting of PD-1, PD-L1, PD-L2, 4-1BB, GITR, CD40, OX40, and ICOS. In another preferred embodiment, said dsRNA is polyIC, said PEI is LPEI covalently linked to one, two or three PEG moieties, said targeting moiety is EGF, preferably human EGF, and said immune checkpoint protein is selected from the group consisting of PD-1, PD-L1, PD-L2, 4-1BB, GITR, CD40, and OX40. In another preferred embodiment, said dsRNA is polyIC, said PEI is LPEI covalently linked to one, two or three PEG moieties, said targeting moiety is EGF, preferably human EGF, and said immune checkpoint protein is selected from the group consisting of PD-1, PD-L1, PD-L2, 4-1BB, GITR and OX40. In another preferred embodiment, said dsRNA is polyIC, said PEI is LPEI covalently linked to one, two or three PEG moieties, said targeting moiety is EGF, preferably human EGF, and said immune checkpoint protein is selected from the group consisting ofPD-1, PD-L1, PD-L2, 4-1BB and OX40.

In another preferred embodiment, said dsRNA is polyIC, said PEI is LPEI covalently linked to one, two or three PEG moieties, said targeting moiety is EGF, preferably human EGF, and said immune checkpoint protein is selected from the group consisting of PD-1, PD-L1, PD-L2, CTLA-4, 4-1BB and 4-1BB ligand. In another preferred embodiment, said dsRNA is polyIC, said PEI is LPEI covalently linked to one, two or three PEG moieties, said targeting moiety is EGF, preferably human EGF, and said immune checkpoint protein is selected from the group consisting of PD-1, PD-L1, PD-L2, CTLA-4, and 4-1BB. In another preferred embodiment, said dsRNA is polyIC, said PEI is LPEI covalently linked to one, two or three PEG moieties, said targeting moiety is EGF, preferably human EGF, and said immune checkpoint protein is selected from the group consisting of PD-1, PD-L1, PD-L2, and 4-1BB. In another preferred embodiment, said dsRNA is polyIC, said PEI is LPEI covalently linked to one, two or three PEG moieties and said targeting moiety is EGF, preferably human EGF, and said immune checkpoint protein is PD-1 or 4-1BB. In another preferred embodiment, said dsRNA is polyIC, said PEI is LPEI covalently linked to one, two or three PEG moieties and said targeting moiety is EGF, preferably human EGF, and said immune checkpoint protein is PD-1, PD-L1, or PD-L2. In another preferred embodiment, said dsRNA is polyIC, said PEI is LPEI covalently linked to one, two or three PEG moieties, said targeting moiety is EGF, preferably human EGF, and said immune checkpoint protein is PD-1. In another preferred embodiment, said dsRNA is polyIC, said PEI is LPEI covalently linked to one, two or three PEG moieties and said targeting moiety is EGF, preferably human EGF, and said immune checkpoint protein is PD-L1. In another preferred embodiment, said dsRNA is polyIC, said PEI is LPEI covalently linked to one, two or three PEG moieties and said targeting moiety is EGF, preferably human EGF, and said immune checkpoint protein is PD-L2. In another preferred embodiment, said dsRNA is polyIC, said PEI is LPEI covalently linked to one, two or three PEG moieties and said targeting moiety is EGF, preferably human EGF, and said immune checkpoint protein is 4-1BB. In another preferred embodiment, said dsRNA is polyIC, said PEI is LPEI covalently linked to one, two or three PEG moieties and said targeting moiety is EGF, preferably human EGF, and said immune checkpoint protein is CTLA-4. In another preferred embodiment, said dsRNA is polyIC, said PEI is LPEI covalently linked to one, two or three PEG moieties and said targeting moiety is EGF, preferably human EGF, and said immune checkpoint protein is LAG-3. In another preferred embodiment, said dsRNA is polyIC, said PEI is LPEI covalently linked to one, two or three PEG moieties and said targeting moiety is EGF, preferably human EGF, and said immune checkpoint protein is TIGIT. In another preferred embodiment, said dsRNA is polyIC, said PEI is LPEI covalently linked to one, two or three PEG moieties and said targeting moiety is EGF, preferably human EGF, and said immune checkpoint protein is TIM3. In another preferred embodiment, said dsRNA is polyIC, said PEI is LPEI covalently linked to one, two or three PEG moieties and said targeting moiety is EGF, preferably human EGF, and said immune checkpoint protein is GITR or GITR ligand. In another preferred embodiment, said dsRNA is polyIC, said PEI is LPEI covalently linked to one, two or three PEG moieties and said targeting moiety is EGF, preferably human EGF, and said immune checkpoint protein is ICOS or ICOS ligand. In another preferred embodiment, said dsRNA is polyIC, said PEI is LPEI covalently linked to one, two or three PEG moieties and said targeting moiety is EGF, preferably human EGF, and said immune checkpoint protein is OX40 or OX40 ligand.

In another preferred embodiment, said dsRNA is polyIC, said PEI is LPEI covalently linked to one, two or three PEG moieties, said targeting moiety is EGF, preferably human EGF, and said immune checkpoint protein modulating antibody is selected from the group consisting of anti-PD-1, anti-PD-L1, anti-PD-L2, anti-CTLA-4, anti-B7-1, anti-B7-2, anti-4-1BB, anti-4-1BB ligand (4-1BBL), anti-TIGIT, anti-LAG3, anti-TIM3, anti-B7-H3, anti-B7-H4, anti-VISTA, anti-CCR4, anti-GITR ligand, anti-GITR, anti-OX40, anti-OX-40L, anti-ICOS, anti-ICOS ligand anti-CD40 and anti-CD40 ligand. In another preferred embodiment, said dsRNA is polyIC, said PEI is LPEI covalently linked to one, two or three PEG moieties, said targeting moiety is EGF, preferably human EGF, and said immune checkpoint protein modulating antibody is selected from the group consisting of anti-PD-1, anti-PD-L1, anti-PD-L2, anti-CTLA-4, anti-B7-1, anti-B7-2, anti-4-1BB, anti-4-1BB ligand, anti-TIGIT, anti-LAG3, anti-TIM-3, anti-GITR, anti-GITR ligand, anti-CD40, anti-CD40 ligand, anti-OX40, anti-OX40 ligand, anti-ICOS ligand and anti-ICOS. In another preferred embodiment, said dsRNA is polyIC, said PEI is LPEI covalently linked to one, two or three PEG moieties, said targeting moiety is EGF, preferably human EGF, and said immune checkpoint protein modulating antibody is selected from the group consisting of anti-PD-1, anti-PD-L1, anti-PD-L2, anti-CTLA-4, anti-4-1BB, anti-4-1BB ligand, anti-TIGIT, anti-LAG3, anti-TIM-3, anti-GITR, anti-GITR ligand, anti-CD40, anti-CD40 ligand, anti-OX40, anti-OX40 ligand, anti-ICOS ligand and anti-ICOS. In another preferred embodiment, said dsRNA is polyIC, said PEI is LPEI covalently linked to one, two or three PEG moieties, said targeting moiety is EGF, preferably human EGF, and said immune checkpoint protein modulating antibody is selected from the group consisting of anti-PD-1, anti-PD-L1, anti-PD-L2, anti-CTLA-4, anti-B7-1, anti-B7-2, anti-4-1BB, anti-4-1BB, anti-TIGIT, anti-LAG3, anti-TIM3, anti-B7-H3, anti-B7-H4, anti-VISTA, anti-CCR4, anti-GITR, anti-OX40, anti-ICOS, and anti-CD40. In another preferred embodiment, said dsRNA is polyIC, said PEI is LPEI covalently linked to one, two or three PEG moieties, said targeting moiety is EGF, preferably human EGF, and said immune checkpoint protein modulating antibody is selected from the group consisting of anti-PD-1, anti-PD-L1, anti-PD-L2, anti-CTLA-4, anti-B7-1, anti-B7-2, anti-4-1BB, anti-TIGIT, anti-LAG3, anti-TIM-3, anti-GITR, anti-CD40, anti-OX40, and anti-ICOS. In another preferred embodiment, said dsRNA is polyIC, said PEI is LPEI covalently linked to one, two or three PEG moieties, said targeting moiety is EGF, preferably human EGF, and said immune checkpoint protein modulating antibody is selected from the group consisting of anti-PD-1, anti-PD-L1, anti-PD-L2, anti-CTLA-4, anti-4-1BB and anti-4-1BB ligand. In another preferred embodiment, said dsRNA is polyIC, said PEI is LPEI covalently linked to one, two or three PEG moieties, said targeting moiety is EGF, preferably human EGF, and said immune checkpoint protein modulating antibody is selected from the group consisting of anti-PD-1, anti-PD-L1, anti-PD-L2, anti-CTLA-4, and anti-4-1BB. In another preferred embodiment, said dsRNA is polyIC, said PEI is LPEI covalently linked to one, two or three PEG moieties, said targeting moiety is EGF, preferably human EGF, and said immune checkpoint protein modulating antibody is selected from the group consisting of anti-PD-1, anti-PD-L1, anti-PD-L2, and anti-4-1BB. In another preferred embodiment, said dsRNA is polyIC, said PEI is LPEI covalently linked to one, two or three PEG moieties and said targeting moiety is EGF, preferably human EGF, and said immune checkpoint protein modulating antibody is anti-PD-1 or anti-4-1BB. In another preferred embodiment, said dsRNA is polyIC, said PEI is LPEI covalently linked to one, two or three PEG moieties and said targeting moiety is EGF, preferably human EGF, and said immune checkpoint protein modulating antibody is anti-PD-1, anti-PD-L1, or anti-PD-L2. In another preferred embodiment, said dsRNA is polyIC, said PEI is LPEI covalently linked to one, two or three PEG moieties, said targeting moiety is EGF, preferably human EGF, and said immune checkpoint protein modulating antibody is anti-PD-1. In another preferred embodiment, said dsRNA is polyIC, said PEI is LPEI covalently linked to one, two or three PEG moieties and said targeting moiety is EGF, preferably human EGF, and said immune checkpoint protein modulating antibody is anti-PD-L1. In another preferred embodiment, said dsRNA is polyIC, said PEI is LPEI covalently linked to one, two or three PEG moieties and said targeting moiety is EGF, preferably human EGF, and said immune checkpoint protein modulating antibody is anti-PD-L2. In another preferred embodiment, said dsRNA is polyIC, said PEI is LPEI covalently linked to one, two or three PEG moieties and said targeting moiety is EGF, preferably human EGF, and said immune checkpoint protein modulating antibody is anti-4-1BB. In another preferred embodiment, said dsRNA is polyIC, said PEI is LPEI covalently linked to one, two or three PEG moieties and said targeting moiety is EGF, preferably human EGF, and said immune checkpoint protein modulating antibody is anti-CTLA-4. In another preferred embodiment, said dsRNA is polyIC, said PEI is LPEI covalently linked to one, two or three PEG moieties and said targeting moiety is EGF, preferably human EGF, and said immune checkpoint protein modulating antibody is anti-LAG-3. In another preferred embodiment, said dsRNA is polyIC, said PEI is LPEI covalently linked to one, two or three PEG moieties and said targeting moiety is EGF, preferably human EGF, and said immune checkpoint protein modulating antibody is anti-TIGIT. In another preferred embodiment, said dsRNA is polyIC, said PEI is LPEI covalently linked to one, two or three PEG moieties and said targeting moiety is EGF, preferably human EGF, and said immune checkpoint protein modulating antibody is anti-TIM3. In another preferred embodiment, said dsRNA is polyIC, said PEI is LPEI covalently linked to one, two or three PEG moieties and said targeting moiety is EGF, preferably human EGF, and said immune checkpoint protein modulating antibody is anti-GITR or anti-GITR ligand. In another preferred embodiment, said dsRNA is polyIC, said PEI is LPEI covalently linked to one, two or three PEG moieties and said targeting moiety is EGF, preferably human EGF, and said immune checkpoint protein modulating antibody is anti-ICOS or ICOS ligand. In another preferred embodiment, said dsRNA is polyIC, said PEI is LPEI covalently linked to one, two or three PEG moieties and said targeting moiety is EGF, preferably human EGF, and said immune checkpoint protein modulating antibody is anti-OX40 or anti-OX40 ligand.

In another preferred embodiment, said dsRNA is polyIC, said PEI is LPEI₂₂ₖ covalently linked to one, two or three PEG_{0.3-8k}, preferably PEG₂ₖ moieties, said targeting moiety is EGF, preferably human EGF, and said immune checkpoint protein is selected from the group consisting of PD-1, PD-L1, PD-L2, CTLA-4, B7-1, B7-2, 4-1BB, 4-1BB ligand (4-1BBL), TIGIT, LAG3, TIM3, B7-H3, B7-H4, VISTA, CCR4, GITR ligand, GITR, OX40, OX-40L, ICOS, ICOS ligand CD40 and CD40 ligand. In another preferred embodiment, said dsRNA is polyIC, said PEI is LPEI₂₂ₖ covalently linked to one, two or three PEG_{0.3-8k}, preferably PEG₂ₖ moieties, said targeting moiety is EGF, preferably human EGF, and said immune checkpoint protein is selected from the group consisting of PD-1, PD-L1, PD-L2, CTLA-4, B7-1, B7-2, 4-1BB, 4-1BB ligand, TIGIT, LAG3, TIM-3, GITR, GITR ligand, CD40, CD40 ligand, OX40, OX40 ligand, ICOS ligand and ICOS.

In another preferred embodiment, said dsRNA is polyIC, said PEI is LPEI₂₂ₖ covalently linked to one, two or three PEG_{0.3-8k}, preferably PEG₂ₖ moieties, said targeting moiety is EGF, preferably human EGF, and said immune checkpoint protein is selected from the group consisting of PD-1, PD-L1, PD-L2, CTLA-4, 4-1BB, 4-1BB ligand (4-1BBL), TIGIT, LAG3, TIM3, B7-H3, B7-H4, VISTA, CCR4, GITR ligand, GITR, OX40, OX-40L, ICOS, ICOS ligand CD40 and CD40 ligand. In another preferred embodiment, said dsRNA is polyIC, said PEI is LPEI₂₂ₖ covalently linked to one, two or three PEG_{0.3-8k}, preferably PEG₂ₖ moieties, said targeting moiety is EGF, preferably human EGF, and said immune checkpoint protein is selected from the group consisting of PD-1, PD-L1, PD-L2, CTLA-4, 4-1BB, 4-1BB ligand, TIGIT, LAG3, TIM-3, GITR, GITR ligand, CD40, CD40 ligand, OX40, OX40 ligand, ICOS ligand and ICOS. In another preferred embodiment, said dsRNA is polyIC, said PEI is LPEI₂₂ₖ covalently linked to one, two or three PEG_{0.3-8k}, preferably PEG₂ₖ moieties, said targeting moiety is EGF, preferably human EGF, and said immune checkpoint protein is selected from the group consisting of PD-1, PD-L1, PD-L2, CTLA-4, B7-1, B7-2, 4-1BB, TIGIT, LAG3, TIM3, B7-H3, B7-H4, VISTA, CCR4, GITR, OX40, ICOS, and CD40. In another preferred embodiment, said dsRNA is polyIC, said PEI is LPEI₂₂ₖ covalently linked to one, two or three PEG_{0.3-8k}, preferably PEG₂ₖ moieties, said targeting moiety is EGF, preferably human EGF, and said immune checkpoint protein is selected from the group consisting of PD-1, PD-L1, PD-L2, CTLA-4, B7-1, B7-2, 4-1BB, TIGIT, LAG3, TIM-3, GITR, CD40, OX40, and ICOS.

In another preferred embodiment, said dsRNA is polyIC, said PEI is LPEI₂₂ₖ covalently linked to one, two or three PEG_{0.3-8k}, preferably PEG₂ₖ moieties, said targeting moiety is EGF, preferably human EGF, and said immune checkpoint protein is selected from the group consisting of PD-1, PD-L1, PD-L2, CTLA-4, 4-1BB, GITR, CD40, OX40, and ICOS. In another preferred embodiment, said dsRNA is polyIC, said PEI is LPEI₂₂ₖ covalently linked to one, two or three PEG_{0.3-8k}, preferably PEG₂ₖ moieties, said targeting moiety is EGF, preferably human EGF, and said immune checkpoint protein is selected from the group consisting of PD-1, PD-L1, PD-L2, 4-1BB, GITR, CD40, and OX40. In another preferred embodiment, said dsRNA is polyIC, said PEI is LPEI₂₂ₖ covalently linked to one, two or three PEG_{0.3-8k}, preferably PEG₂ₖ moieties, said targeting moiety is EGF, preferably human EGF, and said immune checkpoint protein is selected from the group consisting of PD-1, PD-L1, PD-L2, 4-1BB, GITR, and OX40. In another preferred embodiment, said dsRNA is polyIC, said PEI is LPEI₂₂ₖ covalently linked to one, two or three PEG_{0.3-8k}, preferably PEG₂ₖ moieties, said targeting moiety is EGF, preferably human EGF, and said immune checkpoint protein is selected from the group consisting of PD-1, PD-L1, PD-L2, 4-1BB and OX40.

In another preferred embodiment, said dsRNA is polyIC, said PEI is LPEI₂₂ₖ covalently linked to one, two or three PEG_{0.3-8k}, preferably PEG₂ₖ moieties, said targeting moiety is EGF, preferably human EGF, and said immune checkpoint protein is selected from the group consisting of PD-1, PD-L1, PD-L2, CTLA-4, 4-1BB and 4-1BB ligand. In another preferred embodiment, said dsRNA is polyIC, said PEI is LPEI₂₂ₖ covalently linked to one, two or three PEG_{0.3-8k}, preferably PEG₂ₖ moieties, said targeting moiety is EGF, preferably human EGF, and said immune checkpoint protein is selected from the group consisting of PD-1, PD-L1, PD-L2, CTLA-4, and 4-1BB. In another preferred embodiment, said dsRNA is polyIC, said PEI is LPEI₂₂ₖ covalently linked to one, two or three PEG_{0.3-8k}, preferably PEG₂ₖ moieties, said targeting moiety is EGF, preferably human EGF, and said immune checkpoint protein is selected from the group consisting of PD-1, PD-L1, PD-L2, and 4-1BB. In another preferred embodiment, said dsRNA is polyIC, said PEI is LPEI₂₂ₖ covalently linked to one, two or three PEG_{0.3-8k}, preferably PEG₂ₖ moieties and said targeting moiety is EGF, preferably human EGF, and said immune checkpoint protein is PD-1 or 4-1BB. In another preferred embodiment, said dsRNA is polyIC, said PEI is LPEI₂₂ₖ covalently linked to one, two or three PEG_{0.3-8k}, preferably PEG₂ₖ moieties and said targeting moiety is EGF, preferably human EGF, and said immune checkpoint protein is PD-1, PD-L1, or PD-L2. In another preferred embodiment, said dsRNA is polyIC, said PEI is LPEI₂₂ₖ covalently linked to one, two or three PEG_{0.3-8k}, preferably PEG₂ₖ moieties, said targeting moiety is EGF, preferably human EGF, and said immune checkpoint protein is PD-1. In another preferred embodiment, said dsRNA is polyIC, said PEI is LPEI₂₂ₖ covalently linked to one, two or three PEG_{0.3-8k}, preferably PEG₂ₖ moieties and said targeting moiety is EGF, preferably human EGF, and said immune checkpoint protein is PD-L1. In another preferred embodiment, said dsRNA is polyIC, said PEI is LPEI₂₂ₖ covalently linked to one, two or three PEG_{0.3-8k}, preferably PEG₂ₖ moieties and said targeting moiety is EGF, preferably human EGF, and said immune checkpoint protein is PD-L2. In another preferred embodiment, said dsRNA is polyIC, said PEI is LPEI₂₂ₖ covalently linked to one, two or three PEG_{0.3-8k}, preferably PEG₂ₖ moieties and said targeting moiety is EGF, preferably human EGF, and said immune checkpoint protein is 4-1BB. In another preferred embodiment, said dsRNA is polyIC, said PEI is LPEI₂₂ₖ covalently linked to one, two or three PEG_{0.3-8k}, preferably PEG₂ₖ moieties and said targeting moiety is EGF, preferably human EGF, and said immune checkpoint protein is CTLA-4. In another preferred embodiment, said dsRNA is polyIC, said PEI is LPEI₁₅₋₂₅ₖ, preferably LPEI₂₂ₖ, covalently linked to one, two or three PEG_{0.3-8k}, preferably PEG₁₋₃ₖ, further preferably PEG₂ₖ moieties and said targeting moiety is EGF, preferably human EGF, and said immune checkpoint protein is LAG-3. In another preferred embodiment, said dsRNA is polyIC, said PEI is LPEI₁₅₋₂₅ₖ, preferably LPEI₂₂ₖ, covalently linked to one, two or three PEG_{0.3-8k}, preferably PEG₁₋₃ₖ, further preferably PEG₂ₖ moieties and said targeting moiety is EGF, preferably human EGF, and said immune checkpoint protein is TIGIT. In another preferred embodiment, said dsRNA is polyIC, said PEI is LPEI₁₅₋₂₅ₖ, preferably LPEI₂₂ₖ, covalently linked to one, two or three PEG_{0.3-8k}, preferably PEG₁₋₃ₖ, further preferably PEG₂ₖ moieties and said targeting moiety is EGF, preferably human EGF, and said immune checkpoint protein is TIM3. In another preferred embodiment, said dsRNA is polyIC, said PEI is LPEI₁₅₋₂₅ₖ, preferably LPEI₂₂ₖ, covalently linked to one, two or three PEG_{0.3-8k}, preferably PEG₁₋₃ₖ, further preferably PEG₂ₖ moieties and said targeting moiety is EGF, preferably human EGF, and said immune checkpoint protein is GITR or GITR ligand. In another preferred embodiment, said dsRNA is polyIC, said PEI is LPEI₁₅₋₂₅ₖ, preferably LPEI₂₂ₖ, covalently linked to one, two or three PEG_{0.3-8k}, preferably PEG₁₋₃ₖ, further preferably PEG₂ₖ moieties and said targeting moiety is EGF, preferably human EGF, and said immune checkpoint protein is ICOS or ICOS ligand. In another preferred embodiment, said dsRNA is polyIC, said PEI is LPEI₁₅₋₂₅ₖ, preferably LPEI₂₂ₖ, covalently linked to one, two or three PEG_{0.3-8k}, preferably PEG₁₋₃ₖ, further preferably PEG₂ₖ moieties and said targeting moiety is EGF, preferably human EGF, and said immune checkpoint protein is OX40 or OX40 ligand.

In another preferred embodiment, said dsRNA is polyIC, said PEI is LPEI covalently linked to one, two or three PEG moieties, said targeting moiety is EGF, preferably human EGF, and said at least one antibody capable of modulating an immune checkpoint protein is a mixture of (i) an antibody capable of modulating immune checkpoint protein CD27 and at least one antibody capable of modulating an immune checkpoint protein selected from the group consisting of PD-1, PD-L1, and PD-L2;

(ii) an antibody capable of modulating immune checkpoint protein CD40 and at least one antibody capable of modulating an immune checkpoint protein selected from the group consisting of PD-1, PD-L1, PD-L2, and CTLA-4;
(iii) an antibody capable of modulating immune checkpoint protein GITR and at least one antibody capable of modulating an immune checkpoint protein selected from the group consisting of PD-1, PD-L1, PD-L2, and CTLA-4;
(iv) an antibody capable of modulating immune checkpoint protein OX40 and at least one antibody capable of modulating an immune checkpoint protein selected from the group consisting ofPD-1, PD-L1, PD-L2, 4-1BB, and CTLA-4;
(v) an antibody capable of modulating immune checkpoint protein 4-1BB and at least one antibody capable of modulating an immune checkpoint protein selected from the group consisting of PD-1, PD-L1, PD-L2, OX40, LAG-3 and CTLA-4; or
(vi) an antibody capable of modulating immune checkpoint protein ICOS and at least one antibody capable of modulating an immune checkpoint protein selected from the group consisting of PD-1, PD-L1, and PD-L2.

In another preferred embodiment, said dsRNA is polyIC, said PEI is LPEI₂₂ₖ covalently linked to one, two or three PEG_{0.3-8k}, preferably PEG₂ₖ moieties, said targeting moiety is EGF, preferably human EGF, and said at least one antibody capable of modulating an immune checkpoint protein is a mixture of (i) an antibody capable of modulating immune checkpoint protein CD27 and at least one antibody capable of modulating an immune checkpoint protein selected from the group consisting of PD-1, PD-L1, and PD-L2;

(ii) an antibody capable of modulating immune checkpoint protein CD40 and at least one antibody capable of modulating an immune checkpoint protein selected from the group consisting of PD-1, PD-L1, PD-L2, and CTLA-4;
(iii) an antibody capable of modulating immune checkpoint protein GITR and at least one antibody capable of modulating an immune checkpoint protein selected from the group consisting of PD-1, PD-L1, PD-L2, and CTLA-4;
(iv) an antibody capable of modulating immune checkpoint protein OX40 and at least one antibody capable of modulating an immune checkpoint protein selected from the group consisting of PD-1, PD-L1, PD-L2, 4-1BB, and CTLA-4;
(v) an antibody capable of modulating immune checkpoint protein 4-1BB and at least one antibody capable of modulating an immune checkpoint protein selected from the group consisting of PD-1, PD-L1, PD-L2, OX40, LAG-3 and CTLA-4; or
(vi) an antibody capable of modulating immune checkpoint protein ICOS and at least one antibody capable of modulating an immune checkpoint protein selected from the group consisting of PD-1, PD-L1, and PD-L2.

In a preferred embodiment, said composition or kits-of-parts of the invention do not include, i.e. exclude a cancer vaccine or a tumor associated antigen. In a preferred embodiment, said composition or kits-of-parts of the invention includes a cancer vaccine or a tumor associated antigen.

In the polyplex of the invention, said one or more PEG moieties are linked to one of said one or more targeting moieties. In a preferred embodiment, said one or more PEG moieties are directly linked to one of said one or more targeting moieties or linked to one of said one or more targeting moieties via one of said one or more linkers.

In a further preferred embodiment, said linker is selected from —CO—R2-RX—R3 or a peptide moiety consisting of 3 to 7 amino acid residues, wherein R2 is selected from (C₁-C₈)alkylene, (C₂-C₈)alkenylene, (C₂-C₈) alkynylene, (C₆-C₁₀)arylene-diyl, or heteroarylenediyl; RX is absent or —S—; R3 is absent or of the formula

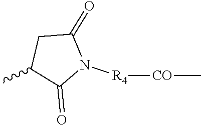

wherein R4 is selected from (C₁-C₈)alkylene, (C₂-C₈)allkenylene, (C₂-C₈) alkynylene(C₁-C₈)alkylene —(C₃-C₈)cycloallkylene, (C₂-C₈)allkenylene-(C₃-C₈)cycloallkylene, (C₂-C₈)alkynylene-(C₃-C₈)cycloallkylene, (C₆-C₁₀)arylene-diyl, heteroarylenediyl, (C₁-C₈)alkylene-(C₆-C₁₀)arylene-diyl, or (C₁-C₈)alkylene-heteroarylenediyl; wherein each one of said (C₁-C₈)allkylene, (C₂-C₈)allkenylene, or (C₂-C₈) alkynylene is optionally substituted by one or more groups each independently selected from halogen, —COR5, —COOR5, —OCOOR5, —OCON(R5)₂, —CN, —NO₂, —SR5, —OR5, —N(R5)₂, —CON(R5)₂, —SO₂R5, —SO₃H, —S(═O)R5, (C₆-C₁₀)aryl, (C₁-C₄)allkylene-(C₆-C₁₀)aryl, heteroaryl, or (C₁-C₄)allkylene-heteroaryl, and further optionally interrupted by one or more identical or different heteroatoms selected from S, O or N, and/or at least one group each independently selected from —NH—CO—, —CO—NH—, —N(R5)—, —N(C₆-C₁₀)aryl-, (C₆-C₁₀)arylene-diyl, or heteroarylenediyl; and R5 is H or (C₁-C₈)alkyl.

In a further preferred embodiment, said polymeric conjugate is of formula (i)-(viii):

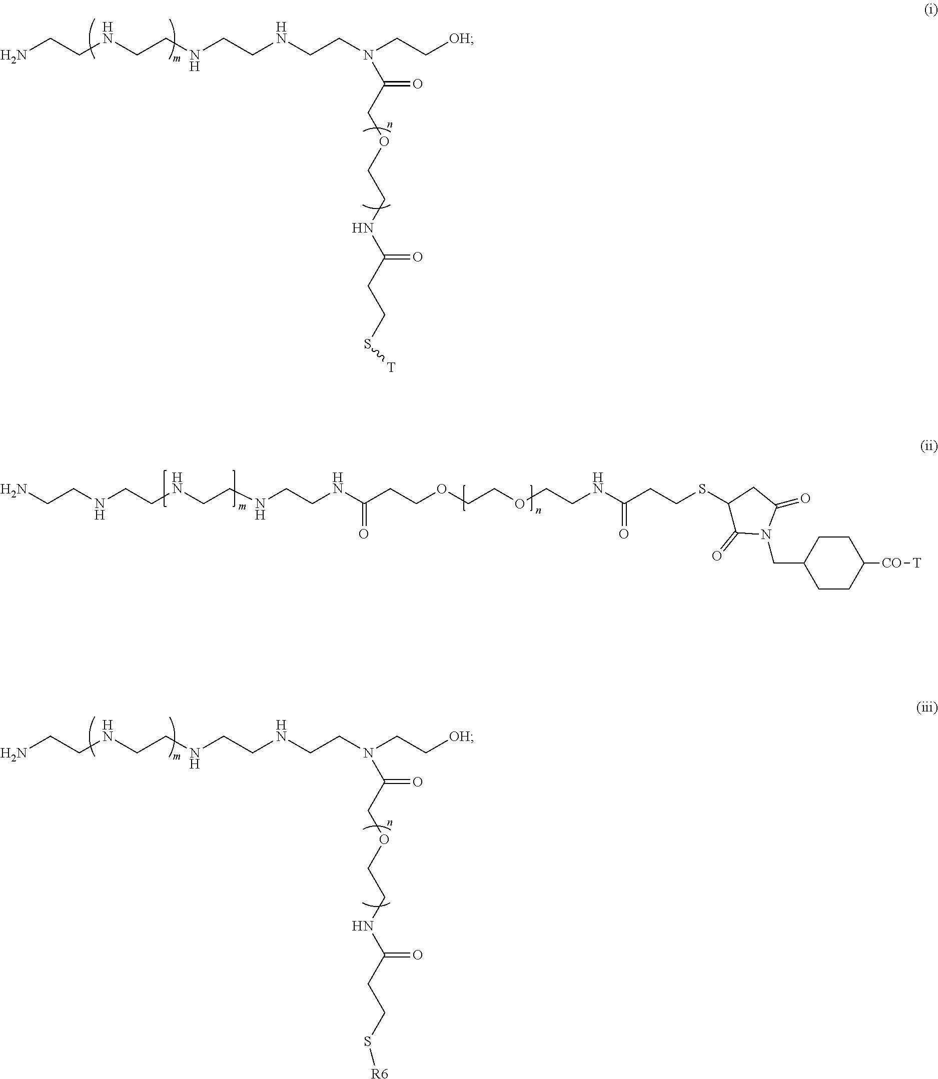

wherein R6 is

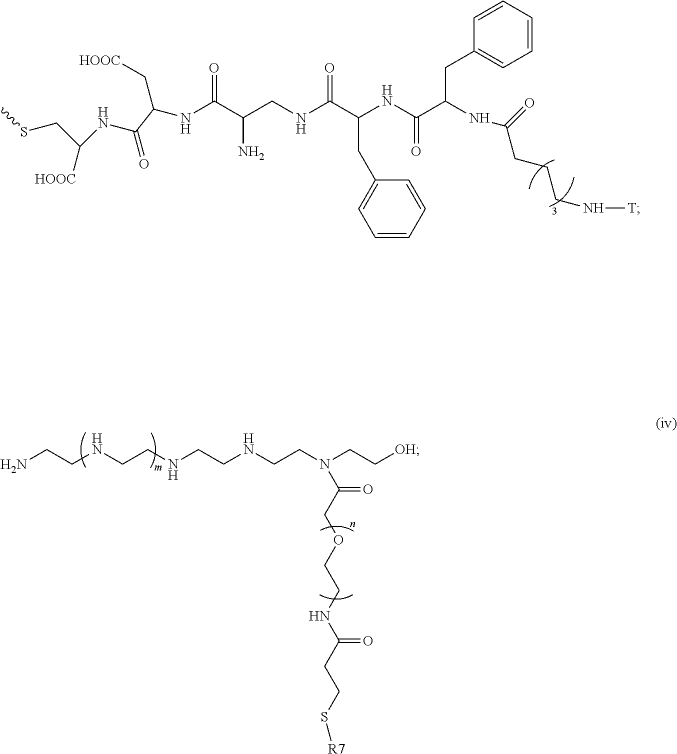

wherein R7 is

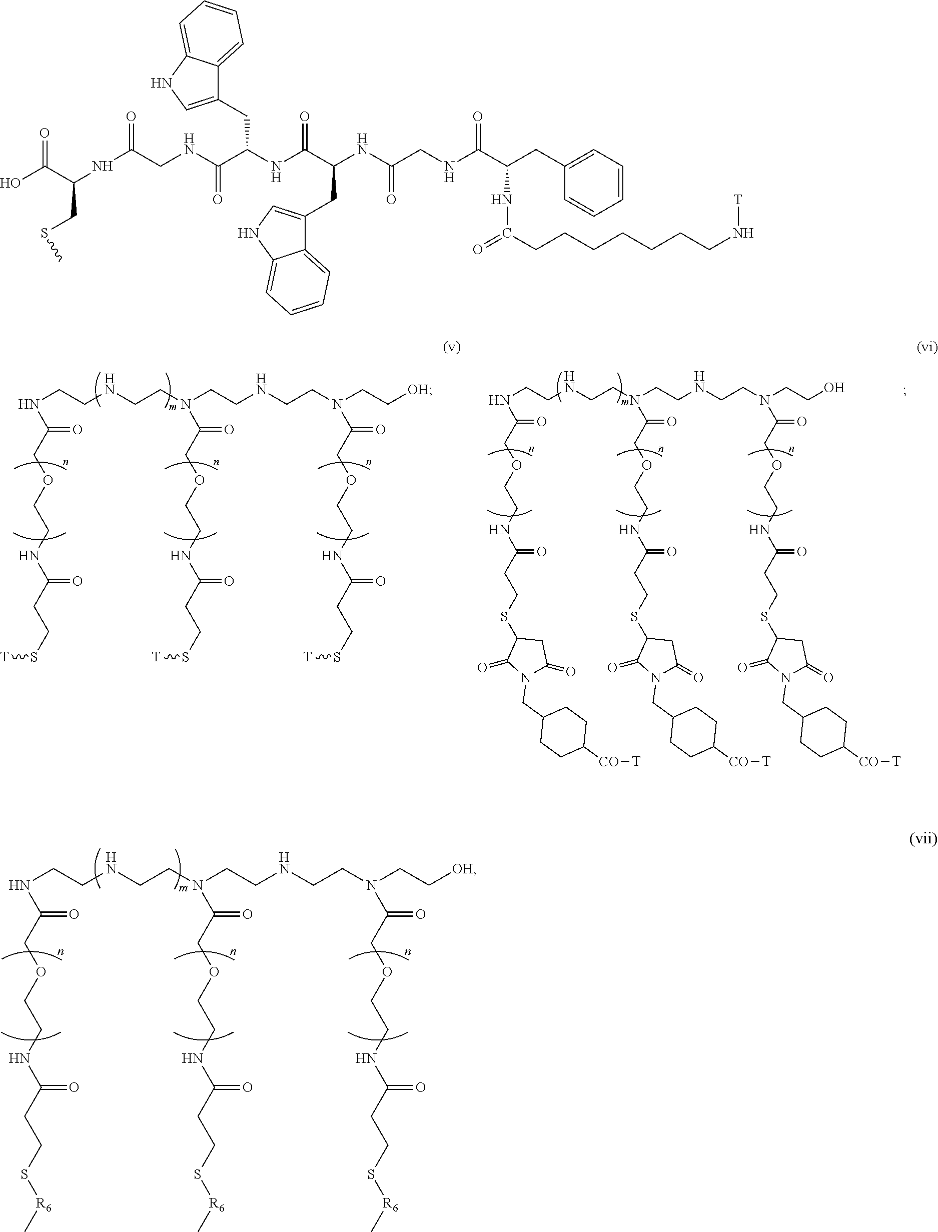

wherein R6 is

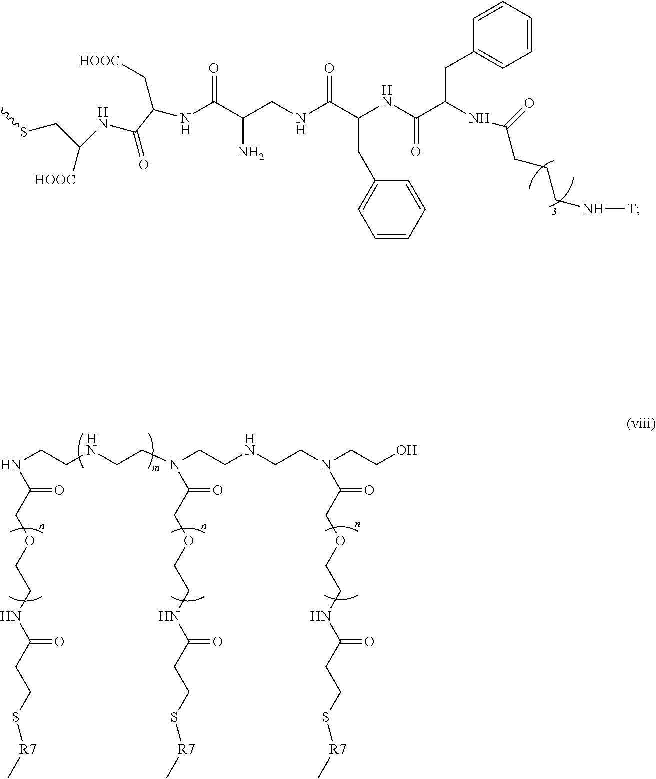

wherein R7 is

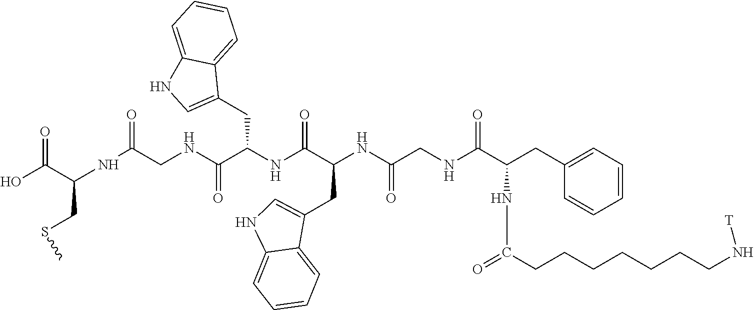

wherein said T represents said targeting moiety, n corresponds to a molecular weight of 0.3-8 kD, preferably 0.5-5 kD, more preferably 1-3 kD, most preferably 2 kD; and m corresponds to a molecular weight of 10-30 kD, preferably 15-25 kD, further preferably 22 kD.

In a further preferred embodiment, said polymeric conjugate is of formula (ii) or (vii), wherein R6 is SEQ ID NO. 4 (—(NH—(CH₂)₇—CO)-Phe-Phe-(NH—CH₂—CH(NH₂)—CO)-Asp-Cys-), and wherein T represent the targeting moiety HOOC(CH₂)₂—CH(COOH)—NH—CO—NH—CH(COOH)—(CH₂)₂—CO— (DUPA moiety). In a further preferred embodiment, said polymeric conjugate is of formula (iv) or (viii), wherein R7 is SEQ ID NO. 3 (—(NH—(CH₂)₇—CO)-Phe-Gly-Trp-Trp-Gly-Cys-), and wherein T represent the targeting moiety HOOC(CH₂)₂—CH(COOH)—NH—CO—NH—CH(COOH)—(CH₂)₂—CO— (DUPA moiety).

In a further preferred embodiment,
said targeting moiety is a HER2 affibody or HER2 antibody, and said polymeric conjugate is of the formula (i) or (v), and the HER2 affibody or HER2 antibody is linked via a mercapto group thereof,
said targeting moiety is hEGF (human epidermal growth factor; human EGF), and said polymeric conjugate is of the formula (i), (ii) or (vi), wherein the hEGF is linked via an amino group thereof, or
said targeting moiety is HOOC(CH₂)₂—CH(COOH)—NH—CO—NH—CH(COOH)—(CH₂)₂—CO— (DUPA moiety), and said polymeric conjugate is of the formula (iii), (iv), (vii) or (viii).

In a further preferred embodiment, said targeting moiety is a HER2 affibody or HER2 antibody, and said polymeric conjugate is of the formula (i) or (v), and the HER2 affibody or HER2 antibody is linked via a mercapto group thereof.

In a further preferred embodiment, said targeting moiety is hEGF, and said polymeric conjugate is of the formula (i), (ii) or (vi). In a further preferred embodiment, said targeting moiety is hEGF, and said polymeric conjugate is of the formula (i), (ii) or (vi), wherein the hEGF is linked via an amino group thereof. In a further preferred embodiment, said targeting moiety is hEGF, and said polymeric conjugate is of the formula (i), wherein the hEGF is linked via an amino group thereof.

In a further preferred embodiment, said targeting moiety is HOOC(CH₂)₂—CH(COOH)—NH—CO—NH—CH(COOH)—(CH₂)₂—CO— (DUPA moiety), and said polymeric conjugate is of the formula (iii), (iv), (vii) or (viii).

In a further preferred embodiment, said targeting moiety is an HER2 affibody or HER2 antibody, and said polymeric conjugate is of the formula (i) or (v), and the HER2 affibody or HER2 antibody is linked via a mercapto group thereof. In a further preferred embodiment, said targeting moiety is hEGF, and said polymeric conjugate is of the formula (i), (ii) or (vi), wherein the hEGF is linked via an amino group thereof. In a further preferred embodiment, said targeting moiety is hEGF, and said polymeric conjugate is of the formula (i), wherein the hEGF is linked via an amino group thereof.

In another preferred embodiment, said dsRNA is polyIC, said targeting moiety is EGF, preferably human EGF, and said immune checkpoint protein is selected from the group consisting of PD-1, PD-L1, PD-L2, CTLA-4, B7-1, B7-2, 4-1BB, 4-1BB ligand (4-1BBL), TIGIT, LAG3, TIM3, B7-H3, B7-H4, VISTA, CCR4, GITR ligand, GITR, OX40, OX-40L, ICOS, ICOS ligand CD40 and CD40 ligand, and wherein said polymeric conjugate is of the formula (i), (ii) or (vi). In another preferred embodiment, said dsRNA is polyIC, said targeting moiety is EGF, preferably human EGF, and said immune checkpoint protein is selected from the group consisting of PD-1, PD-L1, PD-L2, CTLA-4, B7-1, B7-2, 4-1BB, 4-1BB ligand, TIGIT, LAG3, TIM-3, GITR, GITR ligand, CD40, CD40 ligand, OX40, OX40 ligand, ICOS ligand and ICOS, and wherein said polymeric conjugate is of the formula (i), (ii) or (vi).

In another preferred embodiment, said dsRNA is polyIC, said targeting moiety is EGF, preferably human EGF, and said immune checkpoint protein is selected from the group consisting of PD-1, PD-L1, PD-L2, CTLA-4, 4-1BB, 4-1BB ligand (4-1BBL), TIGIT, LAG3, TIM3, B7-H3, B7-H4, VISTA, CCR4, GITR ligand, GITR, OX40, OX-40L, ICOS, ICOS ligand CD40 and CD40 ligand, and wherein said polymeric conjugate is of the formula (i), (ii) or (vi). In another preferred embodiment, said dsRNA is polyIC, said targeting moiety is EGF, preferably human EGF, and said immune checkpoint protein is selected from the group consisting of PD-1, PD-L1, PD-L2, CTLA-4, 4-1BB, 4-1BB ligand, TIGIT, LAG3, TIM-3, GITR, GITR ligand, CD40, CD40 ligand, OX40, OX40 ligand, ICOS ligand and ICOS, and wherein said polymeric conjugate is of the formula (i), (ii) or (vi).

In another preferred embodiment, said dsRNA is polyIC, said targeting moiety is EGF, preferably human EGF, and said immune checkpoint protein is selected from the group consisting of PD-1, PD-L1, PD-L2, CTLA-4, B7-1, B7-2, 4-1BB, TIGIT, LAG3, TIM3, B7-H3, B7-H4, VISTA, CCR4, GITR, OX40, ICOS, and CD40, and wherein said polymeric conjugate is of the formula (i), (ii) or (vi). In another preferred embodiment, said dsRNA is polyIC, said targeting moiety is EGF, preferably human EGF, and said immune checkpoint protein is selected from the group consisting of PD-1, PD-L1, PD-L2, CTLA-4, B7-1, B7-2, 4-1BB, TIGIT, LAG3, TIM-3, GITR, CD40, OX40, and ICOS, and wherein said polymeric conjugate is of the formula (i), (ii) or (vi).

In another preferred embodiment, said dsRNA is polyIC, said targeting moiety is EGF, preferably human EGF, and said immune checkpoint protein is selected from the group consisting of PD-1, PD-L1, PD-L2, CTLA-4, 4-1BB, GITR, CD40, OX40, and ICOS, and wherein said polymeric conjugate is of the formula (i), (ii) or (vi). In another preferred embodiment, said dsRNA is polyIC, said targeting moiety is EGF, preferably human EGF, and said immune checkpoint protein is selected from the group consisting of PD-1, PD-L1, PD-L2, 4-1BB, GITR, CD40, and OX40, and wherein said polymeric conjugate is of the formula (i), (ii) or (vi). In another preferred embodiment, said dsRNA is polyIC, said targeting moiety is EGF, preferably human EGF, and said immune checkpoint protein is selected from the group consisting of PD-1, PD-L1, PD-L2, CTLA-4, 4-1BB, GITR and OX40, and wherein said polymeric conjugate is of the formula (i), (ii) or (vi). In another preferred embodiment, said dsRNA is polyIC, said targeting moiety is EGF, preferably human EGF, and said immune checkpoint protein is selected from the group consisting of PD-1, PD-L1, PD-L2, CTLA-4, 4-1BB and OX40, and wherein said polymeric conjugate is of the formula (i), (ii) or (vi).

In another preferred embodiment, said dsRNA is polyIC, said targeting moiety is EGF, preferably human EGF, and said immune checkpoint protein is selected from the group consisting of PD-1, PD-L1, PD-L2, CTLA-4, 4-1BB and 4-1BB ligand. In another preferred embodiment, said dsRNA is polyIC, said targeting moiety is EGF, preferably human EGF, and said immune checkpoint protein is selected from the group consisting of PD-1, PD-L1, PD-L2, CTLA-4, and 4-1BB. In another preferred embodiment, said dsRNA is polyIC, said targeting moiety is EGF, preferably human EGF, and said immune checkpoint protein is selected from the group consisting of PD-1, PD-L1, PD-L2, and 4-1BB, and wherein said polymeric conjugate is of the formula (i), (ii) or (vi). In another preferred embodiment, said dsRNA is polyIC, said targeting moiety is EGF, preferably human EGF, and said immune checkpoint protein is PD-1 or 4-1BB, and wherein said polymeric conjugate is of the formula (i), (ii) or (vi). In another preferred embodiment, said dsRNA is polyIC, said targeting moiety is EGF, preferably human EGF, and said immune checkpoint protein is PD-1, PD-L1, or PD-L2, and wherein said polymeric conjugate is of the formula (i), (ii) or (vi). In another preferred embodiment, said dsRNA is polyIC, said targeting moiety is EGF, preferably human EGF, and said immune checkpoint protein is PD-1, and wherein said polymeric conjugate is of the formula (i), (ii) or (vi). In another preferred embodiment, said dsRNA is polyIC, said targeting moiety is EGF, preferably human EGF, and said immune checkpoint protein is PD-L1, and wherein said polymeric conjugate is of the formula (i), (ii) or (vi). In another preferred embodiment, said dsRNA is polyIC, said targeting moiety is EGF, preferably human EGF, and said immune checkpoint protein is PD-L2, and wherein said polymeric conjugate is of the formula (i), (ii) or (vi). In another preferred embodiment, said dsRNA is polyIC, said targeting moiety is EGF, preferably human EGF, and said immune checkpoint protein is 4-1BB. In another preferred embodiment, said dsRNA is polyIC, said targeting moiety is EGF, preferably human EGF, and said immune checkpoint protein is CTLA-4, and wherein said polymeric conjugate is of the formula (i), (ii) or (vi). In another preferred embodiment, said dsRNA is polyIC, said targeting moiety is EGF, preferably human EGF, and said immune checkpoint protein is LAG-3, and wherein said polymeric conjugate is of the formula (i), (ii) or (vi). In another preferred embodiment, said dsRNA is polyIC, said targeting moiety is EGF, preferably human EGF, and said immune checkpoint protein is TIGIT. In another preferred embodiment, said dsRNA is polyIC, said targeting moiety is EGF, preferably human EGF, and said immune checkpoint protein is TIM3, and wherein said polymeric conjugate is of the formula (i), (ii) or (vi). In another preferred embodiment, said dsRNA is polyIC, said targeting moiety is EGF, preferably human EGF, and said immune checkpoint protein is GITR or GITR ligand, and wherein said polymeric conjugate is of the formula (i), (ii) or (vi). In another preferred embodiment, said dsRNA is polyIC, and said targeting moiety is EGF, preferably human EGF, and said immune checkpoint protein is ICOS or ICOS ligand, and wherein said polymeric conjugate is of the formula (i), (ii) or (vi). In another preferred embodiment, said dsRNA is polyIC, said targeting moiety is EGF, preferably human EGF, and said immune checkpoint protein is OX40 or OX40 ligand, and wherein said polymeric conjugate is of the formula (i), (ii) or (vi).

In another preferred embodiment, said dsRNA is polyIC, said targeting moiety is EGF, preferably human EGF, and said immune checkpoint protein modulating antibody is selected from the group consisting of anti-PD-1, anti-PD-L1, anti-PD-L2, anti-CTLA-4, anti-B7-1, anti-B7-2, anti-4-1BB, anti-4-1BB ligand (4-1BBL), anti-TIGIT, anti-LAG3, anti-TIM3, anti-B7-H3, anti-B7-H4, anti-VISTA, anti-CCR4, anti-GITR ligand, anti-GITR, anti-OX40, anti-OX-40L, anti-ICOS, anti-ICOS ligand, anti-CD40 and anti-CD40 ligand, and wherein said polymeric conjugate is of the formula (i), (ii) or (vi). In another preferred embodiment, said dsRNA is polyIC, said targeting moiety is EGF, preferably human EGF, and said immune checkpoint protein modulating antibody is selected from the group consisting of anti-PD-1, anti-PD-L1, anti-PD-L2, anti-CTLA-4, anti-B7-1, anti-B7-2, anti-4-1BB, anti-4-1BB ligand, anti-TIGIT, anti-LAG3, anti-TIM-3, anti-GITR, anti-GITR ligand, anti-CD40, anti-CD40 ligand, anti-OX40, anti-OX40 ligand, anti-ICOS ligand and anti-ICOS, and wherein said polymeric conjugate is of the formula (i), (ii) or (vi). In another preferred embodiment, said dsRNA is polyIC, said targeting moiety is EGF, preferably human EGF, and said immune checkpoint protein modulating antibody is selected from the group consisting of anti-PD-1, anti-PD-L1, anti-PD-L2, anti-CTLA-4, anti-4-1BB, anti-4-1BB ligand (4-1BBL), anti-TIGIT, anti-LAG3, anti-TIM3, anti-B7-H3, anti-B7-H4, anti-VISTA, anti-CCR4, anti-GITR ligand, anti-GITR, anti-OX40, anti-OX-40L, anti-ICOS, anti-ICOS ligand, anti-CD40 and anti-CD40 ligand, and wherein said polymeric conjugate is of the formula (i), (ii) or (vi). In another preferred embodiment, said dsRNA is polyIC, said targeting moiety is EGF, preferably human EGF, and said immune checkpoint protein modulating antibody is selected from the group consisting of anti-PD-1, anti-PD-L1, anti-PD-L2, anti-CTLA-4, anti-4-1BB, anti-4-1BB ligand, anti-TIGIT, anti-LAG3, anti-TIM-3, anti-GITR, anti-GITR ligand, anti-CD40, anti-CD40 ligand, anti-OX40, anti-OX40 ligand, anti-ICOS ligand and anti-ICOS, and wherein said polymeric conjugate is of the formula (i), (ii) or (vi).

In another preferred embodiment, said dsRNA is polyIC, said targeting moiety is EGF, preferably human EGF, and said immune checkpoint protein modulating antibody is selected from the group consisting of anti-PD-1, anti-PD-L1, anti-PD-L2, anti-CTLA-4, anti-B7-1, anti-B7-2, anti-4-1BB, anti-TIGIT, anti-LAG3, anti-TIM3, anti-B7-H3, anti-B7-H4, anti-VISTA, anti-CCR4, anti-GITR, anti-OX40, anti-ICOS, and anti-CD40, and wherein said polymeric conjugate is of the formula (i), (ii) or (vi). In another preferred embodiment, said dsRNA is polyIC, said targeting moiety is EGF, preferably human EGF, and said immune checkpoint protein modulating antibody is selected from the group consisting of anti-PD-1, anti-PD-L1, anti-PD-L2, anti-CTLA-4, anti-B7-1, anti-B7-2, anti-4-1BB, anti-TIGIT, anti-LAG3, anti-TIM-3, anti-GITR, anti-CD40, anti-OX40, and anti-ICOS, and wherein said polymeric conjugate is of the formula (i), (ii) or (vi).

In another preferred embodiment, said dsRNA is polyIC, said targeting moiety is EGF, preferably human EGF, and said immune checkpoint protein modulating antibody is selected from the group consisting of anti-PD-1, anti-PD-L1, anti-PD-L2, anti-CTLA-4, anti-4-1BB and anti-4-1BB ligand. In another preferred embodiment, said dsRNA is polyIC, said targeting moiety is EGF, preferably human EGF, and said immune checkpoint protein modulating antibody is selected from the group consisting of anti-PD-1, anti-PD-L1, anti-PD-L2, anti-CTLA-4, and anti-4-1BB. In another preferred embodiment, said dsRNA is polyIC, said targeting moiety is EGF, preferably human EGF, and said immune checkpoint protein modulating antibody is selected from the group consisting of anti-PD-1, anti-PD-L1, anti-PD-L2, and anti-4-1BB, and wherein said polymeric conjugate is of the formula (i), (ii) or (vi). In another preferred embodiment, said dsRNA is polyIC, said targeting moiety is EGF, preferably human EGF, and said immune checkpoint protein modulating antibody is anti-PD-1 or anti-4-1BB, and wherein said polymeric conjugate is of the formula (i), (ii) or (vi). In another preferred embodiment, said dsRNA is polyIC, said targeting moiety is EGF, preferably human EGF, and said immune checkpoint protein modulating antibody is anti-PD-1, anti-PD-L1, or anti-PD-L2, and wherein said polymeric conjugate is of the formula (i), (ii) or (vi). In another preferred embodiment, said dsRNA is polyIC, said targeting moiety is EGF, preferably human EGF, and said immune checkpoint protein modulating antibody is PD-1, and wherein said polymeric conjugate is of the formula (i), (ii) or (vi). In another preferred embodiment, said dsRNA is polyIC, said targeting moiety is EGF, preferably human EGF, and said immune checkpoint protein modulating antibody is anti-PD-L1, and wherein said polymeric conjugate is of the formula (i), (ii) or (vi). In another preferred embodiment, said dsRNA is polyIC, said targeting moiety is EGF, preferably human EGF, and said immune checkpoint protein modulating antibody is anti-PD-L2, and wherein said polymeric conjugate is of the formula (i), (ii) or (vi). In another preferred embodiment, said dsRNA is polyIC, said targeting moiety is EGF, preferably human EGF, and said immune checkpoint protein modulating antibody is anti-4-1BB. In another preferred embodiment, said dsRNA is polyIC, said targeting moiety is EGF, preferably human EGF, and said immune checkpoint protein modulating antibody is anti-CTLA-4, and wherein said polymeric conjugate is of the formula (i), (ii) or (vi). In another preferred embodiment, said dsRNA is polyIC, said targeting moiety is EGF, preferably human EGF, and said immune checkpoint protein modulating antibody modulating antibody is anti-LAG-3, and wherein said polymeric conjugate is of the formula (i), (ii) or (vi).

In another preferred embodiment, said dsRNA is polyIC, said polymeric conjugate is of the formula (i), (ii) or (vi), said targeting moiety is EGF, preferably human EGF, and said at least one antibody capable of modulating an immune checkpoint protein is a mixture of (i) an antibody capable of modulating immune checkpoint protein CD27 and at least one antibody capable of modulating an immune checkpoint protein selected from the group consisting of PD-1, PD-L1, and PD-L2;

(ii) an antibody capable of modulating immune checkpoint protein CD40 and at least one antibody capable of modulating an immune checkpoint protein selected from the group consisting of PD-1, PD-L1, PD-L2, and CTLA-4;
(iii) an antibody capable of modulating immune checkpoint protein GITR and at least one antibody capable of modulating an immune checkpoint protein selected from the group consisting of PD-1, PD-L1, PD-L2, and CTLA-4;
(iv) an antibody capable of modulating immune checkpoint protein OX40 and at least one antibody capable of modulating an immune checkpoint protein selected from the group consisting ofPD-1, PD-L1, PD-L2, 4-1BB, and CTLA-4;
(v) an antibody capable of modulating immune checkpoint protein 4-1BB and at least one antibody capable of modulating an immune checkpoint protein selected from the group consisting of PD-1, PD-L1, PD-L2, OX40, LAG-3 and CTLA-4; or
(vi) an antibody capable of modulating immune checkpoint protein ICOS and at least one antibody capable of modulating an immune checkpoint protein selected from the group consisting of PD-1, PD-L1, and PD-L2.

In another preferred embodiment, said dsRNA is polyIC, said polymeric conjugate is of the formula (i), (ii) or (vi), said targeting moiety is EGF, preferably human EGF, and said at least one antibody capable of modulating an immune checkpoint protein is a mixture of (i) anti-CD27 and at least one antibody selected from the group consisting of anti-PD-1, anti-PD-L1, and anti-PD-L2;

(ii) an antibody capable of modulating immune checkpoint protein anti-CD40 and at least one antibody selected from the group consisting of anti-PD-1, anti-PD-L1, anti-PD-L2, and anti-CTLA-4;
(iii) an antibody capable of modulating immune checkpoint protein anti-GITR and at least one antibody selected from the group consisting of anti-PD-1, anti-PD-L1, anti-PD-L2, and anti-CTLA-4;
(iv) an antibody capable of modulating immune checkpoint protein anti-OX40 and at least one antibody selected from the group consisting of anti-PD-1, anti-PD-L1, anti-PD-L2, anti-4-1BB, and anti-CTLA-4;
(v) an antibody capable of modulating immune checkpoint protein anti-4-1BB and at least one antibody selected from the group consisting of anti-PD-1, anti-PD-L1, anti-PD-L2, anti-OX40, anti-LAG-3 and anti-CTLA-4; or
(vi) an antibody capable of modulating immune checkpoint protein anti-ICOS and at least one antibody selected from the group consisting of anti-PD-1, anti-PD-L1, and anti-PD-L2.

The polyplex of the invention comprises one or more targeting moieties. Said targeting moiety may be a native, natural or modified ligand or a paralog thereof, or a non-native ligand such as an antibody, a single-chain variable fragment (scFv), or an antibody mimetic such as an affibody or aptamer to any one of the cancer antigens.

In a preferred embodiment, said one or more targeting moieties of the polyplex of the invention are selected from the group consisting of EGF, a HER2 affibody, a HER2 antibody, and a DUPA moiety (HOOC(CH₂)₂—CH(COOH)—NH—CO—NH—CH(COOH)—(CH₂)₂—CO—). In another preferred embodiment, said one or more targeting moieties of the polyplex of the invention are EGF, preferably human EGF (hEGF), a HER2 affibody or a HER2 antibody. In another preferred embodiment, said one or more targeting moieties of the polyplex of the invention are EGF, preferably human EGF (hEGF), or a HER2 affibody.

In another preferred embodiment, said one or more targeting moieties of the polyplex of the invention are EGF, preferably human EGF (hEGF), HER2 antibody or a HER2 affibody, and said immune checkpoint protein is selected from the group consisting of 4-1BB, PD-1, PD-L1, PD-L2, CTLA-4, ICOS, CD40, GITR and OX 40. In another preferred embodiment, said one or more targeting moieties of the polyplex of the invention are EGF, preferably human EGF (hEGF), HER2 antibody or a HER2 affibody, and said immune checkpoint protein is selected from the group consisting of 4-1BB, PD-1, PD-L1, PD-L2, CTLA-4, CD40, GITR and OX 40. In another preferred embodiment, said one or more targeting moieties of the polyplex of the invention are EGF, preferably human EGF (hEGF), HER2 antibody or a HER2 affibody, and said immune checkpoint protein is selected from the group consisting of 4-1BB, PD-1, PD-L1, PD-L2, GITR and OX 40. In another preferred embodiment, said one or more targeting moieties of the polyplex of the invention are EGF, preferably human EGF (hEGF), HER2 antibody or a HER2 affibody, and said immune checkpoint protein is selected from the group consisting of 4-1BB, PD-1, PD-L1, PD-L2 and OX 40. In another preferred embodiment, said one or more targeting moieties of the polyplex of the invention are EGF, preferably human EGF (hEGF), HER2 antibody or a HER2 affibody, and said immune checkpoint protein is selected from the group consisting of 4-1BB, PD-1, PD-L1, PD-L2 and CTLA-4. In another preferred embodiment, said one or more targeting moieties of the polyplex of the invention are EGF, preferably human EGF (hEGF), or a HER2 affibody, and said immune checkpoint protein is selected from the group consisting of 4-1BB, PD-1, PD-L1, PD-L2 and CTLA-4.

In more preferred embodiment, said one or more targeting moieties of the polyplex of the invention are EGF, preferably human EGF (hEGF), HER 2 antibody or a HER2 affibody, and said immune checkpoint protein is selected from the group consisting of 4-1BB, PD-1, PD-L1, and PD-L2. In more preferred embodiment, said one or more targeting moieties of the polyplex of the invention are EGF, preferably human EGF (hEGF), or a HER2 affibody, and said immune checkpoint protein is selected from the group consisting of 4-1BB, PD-1, PD-L1, and PD-L2.

In an again more preferred embodiment, said one or more targeting moieties of the polyplex of the invention are EGF, preferably human EGF (hEGF), HER2 antibody or a HER2 affibody, and said immune checkpoint protein is 4-1BB or PD-1. In an again more preferred embodiment, said one or more targeting moieties of the polyplex of the invention are EGF, preferably human EGF (hEGF), or a HER2 affibody, and said immune checkpoint protein is 4-1BB or PD-1.

In a further preferred embodiment, said one or more targeting moieties are EGF including mouse and human EGF. In a more preferred embodiment, said one or more targeting moieties are hEGF. In another preferred embodiment, said one or more targeting moieties of the polyplex of the invention are hEGF of SEQ ID NO: 2.

In another preferred embodiment, said one or more targeting moieties of the polyplex of the invention is a HER2 affibody or a HER2 antibody, preferably HER2 affibody. In another preferred embodiment, said one or more targeting moieties of the polyplex of the invention are a HER2 affibody, preferably said HER2 affibody is of SEQ ID NO: 1. In another preferred embodiment, said one or more targeting moieties of the polyplex of the invention are EGF, preferably human EGF (hEGF) or a DUPA moiety (HOOC(CH₂)₂—CH(COOH)—NH—CO—NH—CH(COOH)—(CH₂)₂—CO—). In another preferred embodiment, said one or more targeting moieties of the polyplex of the invention are a DUPA moiety (HOOC(CH₂)₂—CH(COOH)—NH—CO—NH—CH(COOH)—(CH₂)₂—CO—).

In a further aspect, the invention relates to the composition or the kit-of-parts according to the invention for use in the treatment of cancer.

In a preferred embodiment, the invention relates to the composition or the kit-of-parts according to the invention for use in the treatment of cancer in a mammal. Preferably, said mammal is a human.

In a preferred embodiment, the invention relates to a method for treatment of cancer, wherein said composition or said kit-of-parts according to the invention is administered to a patient in need thereof.

In a preferred embodiment, said cancer is selected from the group consisting of melanoma, non-small-cell lung cancer, breast cancer, ovarian cancer, cervical cancer, endometrial cancer, vulva cancer, urothelial cancer, bladder cancer, renal cancer, esophageal cancer, gastric cancer, pancreatic cancer, colorectal cancer, glial tumor, head and neck cancer, prostate cancer, penile cancer, testicular-embryonal cancer, neuroendocrine tumor, and hepatocellular cancer. In a preferred embodiment, said polyplex included in the kit-of-parts according to the invention is administered separately from the one or more antibodies capable of modulating an immune checkpoint protein.

In one embodiment, the invention provides use of the pharmaceutical composition or kit-of-parts of the invention for the treatment of cancer, wherein said polyplex is administered to a patient in a therapeutically effective amount in combination with a therapeutically effective amount of said at least one antibody capable of modulating an immune checkpoint protein.

In a preferred embodiment, use of the kit-of-parts of the invention in the treatment of cancer comprises independent dosing of the polyplex and said at least one antibody capable of modulating an immune checkpoint protein. Said polyplex and said at least one antibody capable of modulating an immune checkpoint protein can be administered simultaneously or sequentially (consecutive), i.e. chronologically staggered. Said polyplex and said at least one antibody capable of modulating an immune checkpoint protein, as included in the kit-of-parts of the invention, can be combined prior to administration and can be administered together as composition or can be administered separately. In a more preferred embodiment, said polyplex and said at least one antibody capable of modulating an immune checkpoint protein, as included in the kit-of-parts of the invention, are administered separately.

In certain embodiments, the kits-of-parts or the composition for use according to the invention are administered by any suitable route. The kits-of-parts or the composition for use according to the invention may be administered by an intravenous, intra-brain (intracerebral), oral, intramuscular, subcutaneous, transdermal, intradermal, transmucosal, intranasal, sublingual, intraperitoneal or intraocular route. In a preferred embodiment, kits-of-parts or the composition for use according to the invention are systemically administered, i.e. enterally or parenterally. More preferably, kits-of-parts or the composition for use according to the invention are intravenously, subcutaneously or intraperitoneally administered.

In a more preferred embodiment, the kits-of-parts or the composition according to the invention are for systemic administration. More preferably, kits-of-parts or the composition according to the invention are intravenously or intraperitoneally administered, again more preferably intravenously administered.

Said polyplex and said at least one antibody capable of modulating an immune checkpoint protein of the kits-of-parts according to the invention can be administered via the same route or preferably via different routes. More preferably, said polyplex and said at least one antibody capable of modulating an immune checkpoint protein of the kits-of-parts according to the invention are administered via the same route routes. In a preferred embodiment, said polyplex and said at least one antibody capable of modulating an immune checkpoint protein of the kits-of-parts according to the invention are administered sequentially or simultaneously, preferably sequentially. In a preferred embodiment, said polyplex and said at least one antibody capable of modulating an immune checkpoint protein of the kits-of-parts according to the invention are administered sequentially via different routes. More preferably, said polyplex and said at least one antibody capable of modulating an immune checkpoint protein of the kits-of-parts according to the invention are administered simultaneously via the same route routes.

In a preferred embodiment, said polyplex and said at least one antibody capable of modulating an immune checkpoint protein of the kits-of-parts according to the invention are administered sequentially, or simultaneously, preferably sequentially, wherein one compound (or part) of the kits of parts of the invention is administered via intraperitoneal injection and at least one other compound (or part) is administered via intravenous injection. In a preferred embodiment, said polyplex and said at least one antibody capable of modulating an immune checkpoint protein of the kits-of-parts according to the invention are administered sequentially or simultaneously, preferably sequentially, wherein the polyplex is administered via intravenous injection and the immunomodulatory antibodies are administered via intraperitoneal or intravenous injection.

In another preferred embodiment, the polyplex is administered prior to said at least one antibody capable of modulating an immune checkpoint protein. In another preferred embodiment, the polyplex and said at least one antibody capable of modulating an immune checkpoint protein are administered sequentially, wherein the polyplex is administered via intravenous injection and said at least one antibody capable of modulating an immune checkpoint protein are administered via intraperitoneal or intravenous injection, and wherein the polyplex is administered prior to the antibody.

The ratio of the amount or concentration of the polyplex to the amount or concentration the one or more antibody to be administered in the kits-of-parts or composition of the invention can be varied, e.g. in order to cope with the needs of a single patient or a patient sub-population to be treated, wherein the needs can be different due to patient's age, sex, body weight, condition etc.

The kits-of-parts of the invention further may be used as add-on therapy. As used herein, “add-on therapy” means an assemblage of said polyplex and said at least one antibody for use in therapy, wherein the subject receiving the therapy begins a first treatment regimen with one or more different parts of the kits-of-parts prior to beginning a second treatment regimen of one or more different parts of the kits-of-parts in addition to the first treatment regimen, so that not all of the reagents used in the therapy are started at the same time. For example, one or more immunomodulatory antibodies are administered to a patient already receiving the polyplex of the invention or vice versa.

The invention will now be illustrated by the following non-limiting examples.

### EXAMPLES

In the following, the invention is further illustrated by way of examples. Hereto preferred polyplexes of the present invention have been used. The synthesis of said preferred polyplex has been conducted as described in WO 2015/173824, and in particular as described in Examples 2, 8, 10 to 13 of WO 2015/173824. The preferred polymeric conjugate comprising EGF as targeting moiety is abbreviated herein as “PEI-PEG-EGF” and the corresponding preferred polyplex, is interchangeably, abbreviated as “PEI-PEG-EGF/polyIC” or “PPE/PIC”. A further preferred polyplex of the present invention and used herein comprises HER2, in particular HER2 affibody as targeting moiety within the polymeric conjugate and polyIC as the double stranded RNA. Said preferred polyplex, is abbreviated herein as “PolyIC/PPHA”.

### Example 1—Effects of PEI-PEG-EGF/polyIC Alone

A431, U87 and MCF7 cells (40,000 cells per well) were treated for 5 hours with PEI-PEG-EGF/polyIC at various concentrations (0.125, 0.25, 0.5, 1 μg/ml).

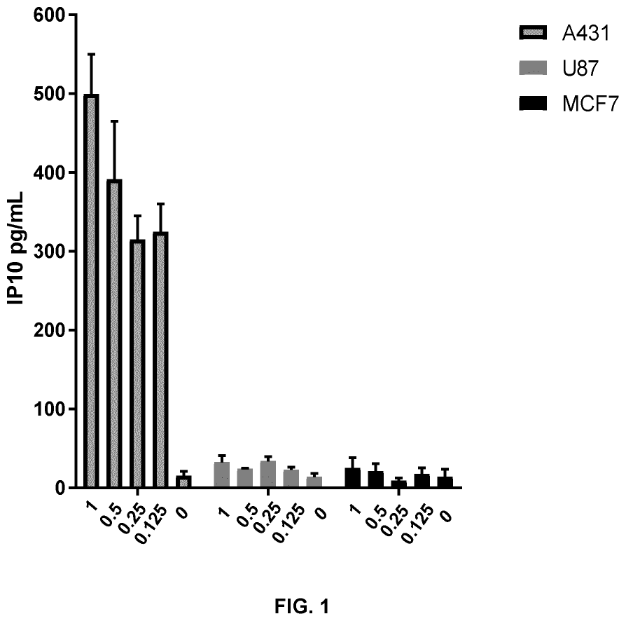

Human IP-10 (CXCL10) secretion was quantified by ELISA assay (ABTS ELISA Development Kit, Peprotech). IP10 secretion by A431 cells expressing high EGFR levels increased strongly after 5 h of incubation with PEI-PEG-EGF/polyIC (FIG. 1).

A431 cells were treated for 5 hours with PEI-PEG-EGF/polyIC at a concentration of 0.125 μg/ml. Then, A431 cells were stained with PD-L1-PE labelled antibody (Biolegend, cat #393607) for 40 minutes on ice in PBS with 2% FCS, then washed and analyzed by FACS instrument. PD-L1 expression significantly increased (MFI=751) after PEI-PEG-EGF/polyIC treatment compared to untreated control cells (MFI=431). Isotype control was used as negative control (MFI=13).

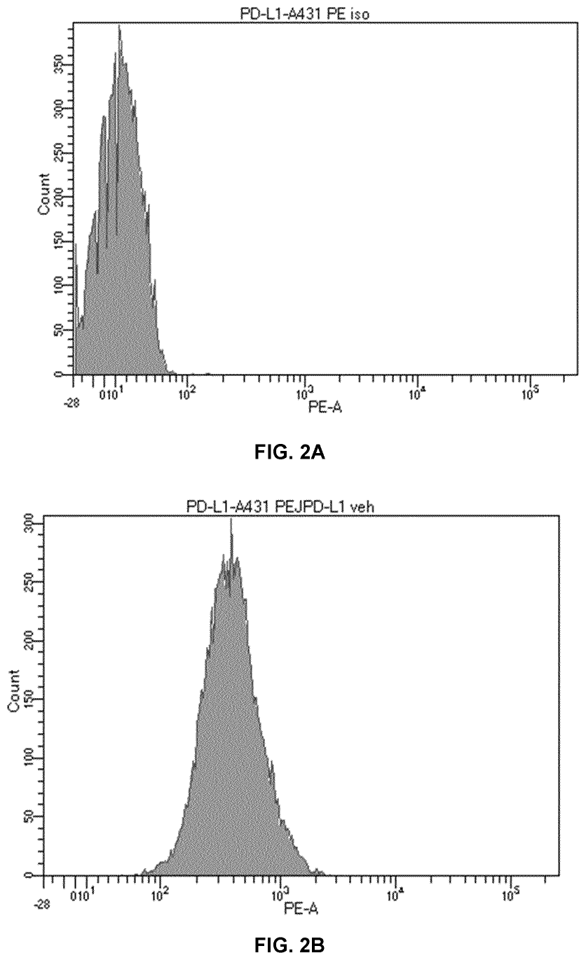

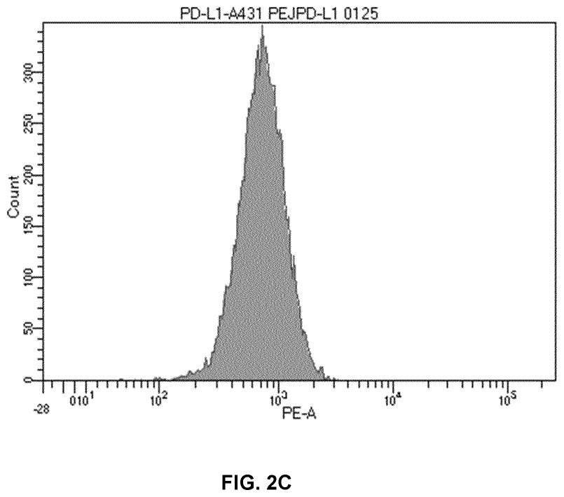

PD-L1 expression was significantly higher after PEI-PEG-EGF/polyIC treatment than in untreated cells (FIG. 2A-C).

Conclusions: Secretion of the potent IFN-y-induced T cell chemokine-IP10 was strongly induced in the EGFR over-expressing cell line A431, after PEI-PEG-EGF/polyIC stimulation. Furthermore, in the low EGFR expressing cell lines, MCF7 and U87, IP10 levels were unaffected. The increase in IP10 after PEI-PEG-EGF/polyIC stimulation provided the rationale to address the immunostimulatory properties of the inventive combination.

In addition, PD-L1 expression was significantly higher after PEI-PEG-EGF/polyIC treatment (MFI=751) compared to untreated cells (MFI=431), providing the rationale for developing the combination with nivolumab (FIG. 2).

### Example 2—In Vitro Effects of PEI-PEG-EGF/polyIC in Combination with Immunomodulatory Therapies Nivolumab and 4-1BB Antibodies

Nivolumab: 40.000 of A431 cells were treated with PEI-PEG-EGF/polyIC at concentration of 0.125, 0.25, 0.5, 1 μg/ml for 5 hours. Then 200.000 PBMCs were stimulated with CD3 (5 μg/ml) and challenged with the diluted medium (1:2) containing PEI-PEG-EGF/polyIC alone (0.125 μg/ml) or in combination with Nivolumab (20 μg/ml) for 48 hours. After 48 hours, the medium from the challenged PBMCs was collected and the INFy production was measured by ELISA assays (FIG. 3).

Combining PEI-PEG-EGF/polyIC with Nivolumab significantly increased IFN-y production by PBMCs (FIG. 3).

4-1BB antibody: 40,000 A431 cells were treated with PEI-PEG-EGF/polyIC at a concentration of 0.5 μg/ml for 5 hours. 200,000 PBMCs from healthy donors were stimulated with CD3 (0.5 μg/ml) or not stimulated and co-cultured with A431 cells treated with PEI-PEG-EGF/polyIC alone or in combination with antibodies against 4-1BB (Biolegend clone, 4B4-1; 10 μg/ml) for 16 hours. After 16 hours the medium was collected and IFN-y was measured by ELISA assays.

Combination of PEI-PEG-EGF/polyIC with anti-4-1BB significantly increased IFN-y production by PBMCs (FIG. 4).

Conclusions: Exposing PBMCs to medium from EGFR overexpressing A431 cells that were treated with PEI-PEG-EGF/polyIC, or culturing the PBMCs in the presence of PEI-PEG-EGF/polyIC-treated A431 cells, induced the PBMCs to secrete IFN-y. Adding either Nivolumab or anti-41BB antibodies led to a further increase in IFN-y.

Therefore, the combination of PEI-PEG-EGF/polyIC with immunotherapy leads to heightened activation of the immune system and more potent antitumor activity. From a clinical point of view, the potential induction of an antitumor immune response upon combined treatment of PEI-PEG-EGF/polyIC with immunological strategies is highly attractive since such combinations should increase percentage of patients responding to immunotherapies and decrease the incidence of resistance development.

### Example 3—In Vivo Efficacy of the Combination of PEI-PEG-mEGF/polyIC+Anti-PD-1

The effect of PEI-PEG-EGF/polyIC+anti-PD-1 on RencaEGFR lung metastases in immunocompetent mice was examined.

Material and Methods: Cells: RencaEGFR; polyplex PEI-PEG-EGF/polyIC with -LPEI/EGF mouse: 1/0.75); poly IC from Dalton; polyplexes in HBG (Hepes Buffered Glucose) at N/P: 8. Anti-PD-1: Rat IgG2a, κ anti mouse PD-1 antibody (Bioxcell clone RMP1-14).

250,000 RencaEGFR cells were injected IV into 40 Balb/c immunocompetent female mice (6 weeks old, weight: 18-21 gr) in order to induce formation of RencaEGFR tumors in lungs. 10 days later animals were divided into 4 groups, 10 animals/group (untreated (UT), antiPD-1, PEI-PEG-EGF/polyIC and PEI-PEG-EGF/polyIC+aPD1 (anti-PD-1).

Mice bearing RencaEGFR tumors were treated IV with PEI-PEG-EGF/polyIC alone at 250 μg/kg, 6 injections/week for 2 weeks or in combination with anti-PD-1 (RPM1-14, rat IgG2a, Biox Cell) at 10 mg/kg IP at day 0, 2, 4, 7, 10. Survival was analyzed afterwards. Appearance of clinical symptoms of cancer: Decrease in weight 10% between the 2 last weighing or 20% between the 1st and the last weighing; slow/abnormal movement; curved back; abnormal breathing justify sacrifice of the mice. Presence of tumor in lungs was verified visually in the sacrificed mice. Mice were sacrificed accordingly to scored parameters as body weight measurements, general healthy conditions of the mice. After the sacrifice of the mice the organs were collected and will be analyzed for IHC to detect the presence of lung metastases.

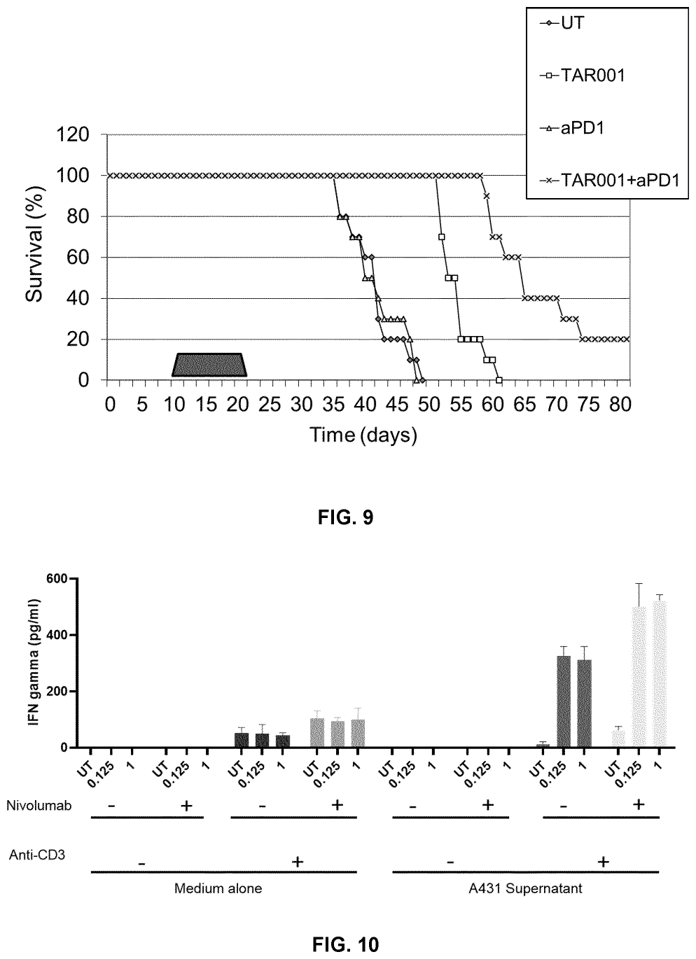

Conclusions: There is 100% uptake of the tumors in two groups (untreated (UT), antiPD-1) with final average survival of 41.5 and 41.8 days. All mice developed tumors after the cell injection which shows high reliability of the model. Anti-PD-1 alone did not inhibit tumor growth. One animal is left alive in PEI-PEG-EGF/polyIC (PPE/PIC) alone group at day 60. Preliminary increase of survival with PEI-PEG-EGF/polyIC is ˜32%. Just 3 animals died in PEI-PEG-EGF/polyIC+anti-PD-1 (PPE/PIC+aPD1) group. Preliminary increase in survival is ˜63% already suggesting synergy between PEI-PEG-EGF/polyIC and anti-PD-1 (FIG. 9).

TABLE 1

| | UT | aPD1 | PPE/PIC | PPE/PIC + aPD1 |
|---|---|---|---|---|
| Average survival (days) | 41.5 | 41.8 | 54.8 | 67.8 |
| (%) Increase survival | | 0.7 | 32.0 | 63.4 |

### Example 4—In Vivo Efficacy of the Combination of PEI-PEG-EGF/polyIC+Anti-CTLA-4

As described in Example 3, RencaEGFR cells are injected IV into 40 Balb/c immunocompetent female mice in order to induce formation of RencaEGFR tumors in lungs. 10 days later animals are divided into 4 groups, untreated control (UT), anti-CTLA-4, PEI-PEG-EGF/polyIC and PEI-PEG-EGF/polyIC+anti-CTLA-4. Mice bearing EGFR tumors are treated with PEI-PEG-EGF/polyIC alone or in combination with anti-CTLA-4 at day 0, 2, 4, 7, 10.

Mice are sacrificed accordingly to scored parameters as body weight measurements, general healthy conditions of the mice. After the sacrifice of the mice the organs will be collected and analyzed for IHC to detect the presence of lung metastases. Survival was analyzed as well as appearance of clinical symptoms of cancer. Presence of tumor in lungs is verified visually in the sacrificed mice.

A reduction in clinical symptoms of cancer, inhibition of tumor growth as well as increase of survival is expected.

### Example 5—Combination Treatment with polyIC/PPHA and Anti-PD-1 Antibody in Immunocompetent Mice Bearing HER-2 Overexpressing Tumors

Materials and Methods: RENCA-HER2 cells (renal cell carcinoma cells which overexpress HER2) (5×10⁶) were injected subcutaneously (s.c.) into the right flanks of immunocompetent BALB/c female mice (6-8 weeks old; Harlan Laboratories). Mice bearing s.c. RENCA HER2 tumors were randomized into 4 groups (untreated, anti-PD-1, polyplex of polyIC/PEI-PEG-HER2 affibody (PPHA) and anti-PD-1+polyIC/PPHA) of 7-8 animals/group with average tumor volume of 235 mm³. PolyIC/PPHA is preferably synthetized as described in the Examples of WO 2015/173824, more preferably Example 2 and pages 18ff of WO 2015/173824. HER2 affibody is preferably synthetized as described on page 20ff of WO 2015/173824.

Mice were treated intravenously (i.v.) with polyIC/PPHA 0.25 mg/kg or 6.25 mg/mouse, N/P 8, every 24 h for 10 days. 4 dosages of anti-PD-1 (BioXCell, InVivoMAb anti-mouse PD-1 (CD279), clone RMP1-14, cat #BE0146), 200 mg/mouse were injected intraperitoneally (i.p.), every 4-6 days. Tumor xenografts were measured with calipers and tumor volumes were determined using the formula: length×width²/2, and plotted as means SEM.

Conclusions: A significant inhibition of tumor growth was observed in both combination arm and polyIC/PPHA alone as compared to anti-PD-1 antibody alone and as compared to untreated tumors. Complete regression of tumors was observed in 2 out of 8 mice in combination arm. These mice did not show any tumor growth for additional 60 days. Cured mice showed complete protection from tumor re-challenge (5×10⁶), indicating that an immune response against the tumor has been generated.

### Example 6—In Vitro Experiment, PD-L1 Expression

Materials and Methods: Increased expression of PD-L1 was shown on RENCA HER2 cells following treatment with polyIC/PPHA. RENCA HER2 cells were treated with polyIC/PPHA at a concentration of 1 microgram/ml for 24 hours. Cells were analyzed using phycoerythrin (PE)-conjugated anti-mouse PD-L1 antibody. After treatment cells were trypsonized, washed and incubated with anti-PD-L1 antibody CD274 (PD-L1, B7-H1) [10F.9G2] Tonbo cat #50-1243-U025, or isotype control for 1 hour. PD-L1 expression was analyzed using flow cytometry (BD FACS ARIAIII; BD Biosciences). Live cells were gated based on SSC and FSC. PE-positive was gated and mean PE was determined.

Conclusions: RENCA HER-2 cells were treated for 24 hours with 1 microgram/ml of polyIC/PPHA and PD-L1 expression was analyzed using flow cytometry. A profound increase in PD-L1 expression was observed following treatment with 1 microgram/ml of polyIC/PPHA as compared to untreated cells, indicating that targeted delivery of polyIC induces upregulation of PD-L1 expression.

TABLE 2

| | Live Cells Counts |
|---|---|
| | Calculated raw values of medians |
| | using x-axis channel(s): PE-A |
| RENCA HER2 Isotype control | 3.19 |
| RENCA HER2 Untreated | 162.53 |
| RENCA HER2 | 321.97 |
| 1 microgram/ml polyIC/PPHA | |

### Example 7—PBMCs are Activated Indirectly by the Polyplex of the Invention Comprising polyIC and a Targeted Polymeric Conjugate

The following experiments demonstrate the benefit of combination of the polyplex of the invention with Nivolumab over single agent treatment.

Materials and Methods: IFN 7 gamma release from PBMCs that were exposed to supernatant of PEI-PEG-EGF/polyIC polyplexes treated A431 cells was quantified. Cell lines: The cell types used are A431 cancer cells (ATCC) and PBMCs (Healthy donor 147). A431 cells were cultured in DMEM medium with 10% fetal calf serum, 100 units/ml penicillin, 100 μg/ml streptomycin. Buffy coats were obtained from healthy donors. PBMCs were isolated from the buffy coats and were cultured in RPMI-1640 medium with 10% fetal calf serum, 100 units/ml penicillin, 100 μg/ml streptomycin.

PEI-PEG-EGF/polyIC polyplexes are prepared in HBG (Hepes Buffered Glucose). PEI-PEG-EGF triconjugate was composed of a PEGylated linear polyethyleneimine conjugated via a MCC linker to hEGF (hEGF-4-(N-Maleimidomethyl)cyclohexane-1-carboxylic acid) and synthetized according to WO 2015/173824, Example 10. Anti-PD-1 antibody (Nivolumab) was utilized either alone or in combination with PEI-PEG-EGF/polyIC polyplex. AntiCD3 antibody (clone OKT3) was used to stimulate PBMCs after transfer of medium from treated A431 cells. Anti-PD-1 antibody (Nivolumab) was used to test the combination of PEI-PEG-EGF/polyIC polyplex and checkpoint blockade. Human interferon 7 secretion was determined with an ELISA kit (BD Bioscience), according to the manufacturer's instructions.

A431 cells were seeded in a flat-bottom 96-well plate (40,000 cells/90 μl DMEM medium). In parallel, a plate with medium alone (90 μl medium without cells) was prepared as a negative control. The next day, A431 cells or medium alone were treated with 0, 0.125 or 1 μg/ml of PEI-PEG-EGF/polyIC polyplex (10 l/well) for 5 hours at 37° C. Frozen PBMCs were thawed and allowed to recover in RPMI-1640 medium at 37° C. for at least 5 h in U-bottom 96-well plates (200,000 cells/100 μl). After 5 h, supernatant (SN) from PEI-PEG-EGF/polyIC polyplex-treated A431 cells or PEI-PEG-EGF/polyIC polyplex-“treated” medium was transferred to the PBMCs. Thereafter, PBMCs were stimulated using anti-CD3 antibody (OKT3, 500 ng/ml) or/and treated with Nivolumab (20 μg/ml) and incubated o/n at 37° C. The SN of stimulated PBMCs was collected and stored at −20° C. until analyzed by ELISA.

To model the effect of combining PEI-PEG-EGF/polyIC polyplex with Nivolumab treatment on immune cell activation in vitro, the SN from A431 cells that had been treated with PEI-PEG-EGF/polyIC polyplex was transferred to human PBMCs from a healthy donor and were treated in combination with Nivolumab, as follows. A431 cells were treated with PEI-PEG-EGF/polyIC polyplex at the indicated concentrations for 5 hours. As a control, medium without cells was “treated” with PEI-PEG-EGF/polyIC polyplex and incubated for 5 hours as well. After 5 hours, PBMCs stimulated or unstimulated with anti-CD3 antibody were treated with the following: Nivolumab alone; SN from PEI-PEG-EGF/polyIC polyplex-treated A431 cells alone; PEI-PEG-EGF/polyIC polyplex-“treated” medium alone; Nivolumab plus SN from PEI-PEG-EGF/polyIC polyplex treated A431 cells; or Nivolumab plus PEI-PEG-EGF/polyIC polyplex-“treated” medium. PBMCs were incubated o/n at 37° C. To assess PBMC activation, IFN-γ secreted from the PBMCs was quantified by ELISA.

Results: As can be seen in FIG. 10 and Table 3, CD3-stimulated PBMCs that received SN from PEI-PEG-EGF/polyIC polyplex-treated A431 cells secreted much more IFN-γ (311.1-326.3 pg/ml) than PBMCs that received SN from untreated (UT) cells (12.7 pg/ml). IFN-γ secretion was further enhanced by the addition of Nivolumab (502-523 pg/ml). PBMCs that received SN from untreated cells plus Nivolumab secreted low levels of IFN-γ (62.5 pg/ml). Nivolumab treatment alone did not induce IFN-γ secretion. PEI-PEG-EGF/polyIC polyplex-“treated” medium (without cells) alone or in combination with Nivolumab did not result in an increase in IFN-γ secretion as compared to UT medium. These results demonstrate that combination of PEI-PEG-EGF/polyIC polyplex with Nivolumab results in increased activation of PBMCs, as seen by increased IFN-γ secretion as compared to PEI-PEG-EGF/polyIC polyplex alone. Moreover, the PBMCs are activated by the cytokines that are secreted from the PEI-PEG-EGF/polyIC polyplex-treated A431 cancer cells, rather than by PEI-PEG-EGF/polyIC polyplex treatment alone.

Conclusions: Combination of the polyplex comprising polyIC and the targeting moiety human EGF and Nivolumab was repeated in a medium transfer experiment following treatment of A431 cells. IFN-γ levels were measured by ELISA and demonstrate that combination of both agents resulted in increase of IFN-γ as compared to single agent alone. As a control, single agents (polyplex or Nivolumab) or the combination were incubated in medium with no cells for the same time course. Medium was then transferred to PBMCs. IFN-γ levels were significant lower in this group as compared to PBMCs receiving medium transferred from A431 treated cells. These results demonstrate that the activation of PBMCs is not mediated directly by polyIC polyplex but rather via the secretion of chemokines from cancer cells treated with the polyIC polyplex.

TABLE 3
Combination of PEI-PEG-EGF/polyIC polyplex and Nivolumab increases PBMC
activation as demonstrated by IFN-γ ELISA

| Medium + PEI-PEG-EGF/polyIC | | | | | A431 SN + PEI-PEG-EGF/polyIC | | | |
|---|---|---|---|---|---|---|---|---|
| polyplex (PPE/PIC) | | | | | polyplex (PPE/PIC) | | | |
| | | | | CD3 + | | | | CD3 + |
| | Medium | Nivolumab | CD3 | Nivolumab | Medium | Nivolumab | CD3 | Nivolumab |
| UT | 0 | 0 | 52.7 | 103.6 | 0 | 0 | 12.7 | 62.5 |
| 0.125 | 0 | 0 | 49.5 | 93.8 | 0 | 0 | 326.3 | 501.6 |
| μg/ml | | | | | | | | |
| PPE/PIC | | | | | | | | |
| 1 μg/ml | 0 | 0 | 43.8 | 99.5 | 0 | 0 | 311.1 | 522.6 |
| PPE/PIC | | | | | | | | |

### Example 8—Secretion of Cytokines Following Treatment with PEI-PEG-EGF/polyIC Polyplex and Each Component Alone

To assess whether PEI-PEG-EGF/polyIC polyplex or its components induce the secretion of pro-inflammatory cytokines, three cell lines, including two high EGFR-expressing lines (MDA-MB-468 and A431) and a low EGFR line (MCF7) were treated with PEI-PEG-EGF/polyIC polyplex, triconjugate PEI-PEG-EGF or pIC and IP-10 GRO-α and CCL5 cytokine secretion was measured by ELISA.

Materials and Methods: Three cell lines (high EGFR expressing-MDA-MB-468 and A431) and low EGFR (MCF7) were treated with PEI-PEG-EGF/polyIC polyplex, PEI-PEG-EGF triconjugate or polyIC at the indicated concentrations for 5 hours. Medium was then collected and analyzed for CCL5 (RANTES), IP10 and GROα. PEI-PEG-EGF was composed of a PEGylated linear polyethyleneimine conjugated via a linker MCC to hEGF, synthesized according to WO 2015/173824, Example 10.

Tumor Cell Lines: MDA-MB-468, A431 and MCF7 cells were provided by the ATCC. Cell lines were routinely passaged once or twice weekly and maintained in culture for up to 20 passages. Cell lines were grown at 37° C. in a humidified atmosphere with 5% CO2 in RPMI-1640 medium (25 mM HEPES, with L-glutamine, #FG1385, Biochrom, Berlin, Germany) or DMEM supplemented with 10% (v/v) fetal calf serum (Sigma, Taufkirchen, Germany), 100 units/ml penicillin and 100 μg/ml streptomycin.

Treatments: PEI-PEG-EGF/polyIC polyplexes or pIC alone were incubated in HBG (HEPES-Buffered Glucose). Reagents were used at concentrations between 0-1 μg/ml of pIC. Triconjugate PEI-PEG-EGF alone was prepared in a similar manner to PEI-PEG-EGF/polyIC polyplexes without pIC.

Quantification of cytokine production by ELISA: To detect IP-10, Gro-α and RANTES secretion, 40,000 cells per well were treated with PEI-PEG-EGF/polyIC polyplexes and pIC concentrations between 0-1 μg/ml, or the equivalent concentrations of PEI-PEG-EGF triconjugate or pIC, for 5 h. Supernatants were collected and cytokine secretion was quantified by IP-10, GROα and RANTES ELISA (Peprotech) using a Synergy H1 plate reader (Biotek).

Results: PEI-PEG-EGF/polyIC polyplex treatment of the high EGFR-expressing cells, MDA-MB-468 and A431, induced the secretion of, IP-10 GRO-α and CCL5, while in the low EGFR-expressing MCF7 cells no cytokine expression was observed (FIGS. 11 A, B and C respectively). Up to 337 and 268 pg/ml of IP10, 496 and 757 pg/ml of GROα and 607 and 100 pg/ml of RANTES were observed in A431 and MDA-MB-468 respectively. No cytokine secretion was observed in all cell lines following treatment with PEI-PEG-EGF triconjugate or pIC alone. These results demonstrate that the PEI-PEG-EGF/polyIC polyplex, but not its individual components, induces the secretion of the pro-inflammatory cytokines.

### Example 9—Activation of PBMCs by PEI-PEG-EGF/polyIC Polyplex, and pIC/PPHA

Materials and Methods: Tumor Cell Lines: MDA-MB-468, A431 and MCF7 cells were provided by the ATCC. Cell lines were routinely passaged once or twice weekly and maintained in culture for up to 20 passages. Cell lines were grown at 37° C. in a humidified atmosphere with 5% CO₂ in RPMI-1640 medium (25 mM HEPES, with L-glutamine, #FG1385, Biochrom, Berlin, Germany) or DMEM supplemented with 10% (v/v) fetal calf serum (Sigma, Taufkirchen, Germany) and 100 units/ml penicillin, 100 μg/ml streptomycin.

Reagents: PEI-PEG-EGF triconjugate was composed of a PEGylated linear polyethyleneimine conjugated via a MCC linker to hEGF (human epidermal growth factor), synthetized according to WO 2015/173824, Example 10. PEI-PEG-EGF/polyIC polyplexes and PEI-PEG-EGF/pLGA polyplex (PEI-PEG-EGF and poly-L-Glutamic-Acid (pLGA), Sigma, 50-100 kDa p4886) were prepared in HBG (HEPES-buffered Glucose) and naked pIC were utilized. Reagents were used at concentrations of 0 to 1 μg/ml of pIC or pLGA within the polyplexes.

PBMC isolation from healthy donors (buffy coats): Buffy coats from healthy donors were obtained from the University Hospital Basel Blood Bank. PBMCs were isolated from the buffy coats and were cultured in RPMI-1640 medium with 10% fetal calf serum, 100 units/ml penicillin, 100 μg/ml streptomycin.

Quantification of cytokine production by ELISA: To quantify IFN-γ and TNF-α secretion, 40,000 cells per well or medium alone were treated with the following compounds: PEI-PEG-EGF/polyIC polyplex at pIC concentrations between 0-1 μg/ml, pIC (0-1 μg/ml) alone, or PEI-PEG-EGF polyplexed with polyglutamic acid at 0-1 μg/ml (PEI-PEG-EGF/pLGA polyplexes). After 5 h of incubation supernatants and medium alone were collected and transferred to 200,000 PBMCs stimulated with a-CD3 (0.5 μg/ml) and incubated for 16 additional h. In parallel, supernatants from each cell line treated with each compound alone were incubated for 16 h in the absence of PBMCs. After incubation, the medium was collected and TNF-α, IFN-γ, and IL-2 were measured by ELISA assays (Peprotech and Invitrogen) and read using a Synergy H1 plate reader from Biotek.

Results: PBMC activation by cytokines secreted from PEI-PEG-EGF/polyIC polyplex-treated cancer cells: Pro-inflammatory cytokines such as IP-10 are known to induce the recruitment and activation of lymphocytes. To assess whether PEI-PEG-EGF/polyIC polyplex or its drug substance pIC induce immune cell activation, three cell lines, high EGFR expressing (MDA-MB-468 and A431) and low EGFR (MCF7) were treated with PEI-PEG-EGF/polyIC polyplex, pIC or PEI-PEG-EGF/pLGA polyplexes (PEI-PEG-EGF polyplexed with polyglutamic acid (pLGA)) at the indicated concentrations. In the PEI-PEG-EGF/pLGA polyplexes, the pIC of PEI-PEG-EGF/polyIC polyplex was replaced with polyglutamic acid to demonstrate that the effect of PEI-PEG-EGF/polyIC polyplex is pIC-mediated. Following 5 h of treatment, supernatant from the treated cancer cells were transferred to PBMCs from healthy donors (FIG. 12; “PBMCs+SN” from each cell line). The PBMCs were then incubated for 16 h and analyzed by ELISA for IFN-γ and TNFα cytokine secretion. In parallel, to demonstrate that PEI-PEG-EGF/polyIC polyplex did not induce the activation of PBMCs directly, PEI-PEG-EGF/polyIC polyplex, pIC or PEI-PEG-EGF/pLGA polyplexes were incubated in medium without cells, and this medium was transferred to PBMCs, which were incubated for 16 h and tested by ELISA (FIG. 12; “PBMCs alone”). As an additional control, supernatants from the treated cancer cells that were incubated for the same time but without PBMCs were also analyzed (FIG. 12; “SN alone”).

Increased secretion of IFN-γ was observed in PBMCs incubated with supernatant from PEI-PEG-EGF/polyIC polyplex-treated MDA-MB-468 and A431. Up to 3587 pg/ml and 2023 pg/ml of IFN-γ were detected in PBMCs from MDA-MB-468 and A431 respectively. PBMCs incubated with supernatant from PEI-PEG-EGF/polyIC polyplex-treated MCF7 cells did not induce any cytokine secretion. PBMCs treated with medium without cells containing PEI-PEG-EGF/polyIC polyplex, pIC or PEI-PEG-EGF/pLGA polyplexes (PBMCs alone) did not induce IFN-γ secretion in all cancer cell lines. No IFN-γ secretion was observed in supernatants from any of the cancer cell lines treated with either PEI-PEG-EGF/polyIC polyplex, pIC or PEI-PEG-EGF/pLGA polyplexes, without PBMCs.

A significant increase in TNFα secretion was observed in PBMCs incubated with media from PEI-PEG-EGF/polyIC polyplex-treated MDA-MB-468 and A431 cells. Up to 2190 pg/ml and 3438 pg/ml of TNFα were detected in PBMCs from MDA-MB-468 and A431 respectively. PBMCs incubated with medium from PEI-PEG-EGF/polyIC polyplex-treated MCF7 cells did not induce cytokine secretion. Medium from PBMCs treated with PEI-PEG-EGF/polyIC polyplex, pIC or PEI-PEG-EGF/pLGA polyplexes (PBMCs alone) did not induce a significant increase in TNFα secretion in all cancer cell lines as compared to UT cells. No secretion of IFN-γ and TNFα was observed in supernatants from cancer cell lines treated with PEI-PEG-EGF/polyIC polyplex, pIC or PEI-PEG-EGF/pLGA polyplexes, without PBMCs.

### Example 10—Efficacy and Selectivity of PEI-PEG-EGF/polyIC Polyplex Compared to Naked pIC, pIC Complexed with PEI or Lipid Based Transfection Reagent

The aim of the study was to measure the selectivity and potency of PEI-PEG-EGF/polyIC polyplexes versus single components and untargeted delivery systems and pIC analogues.

Materials and Methods: Cell lines: High EGFR-expressing cell lines (BT20, MDA-MB-468 and HCC70), medium EGFR-expressing cell line (U87MG), low EGFR-expressing cell lines (MCF7 and U138) and non-cancer cell lines (WI-38 and MCF10A) were used for the experiments. Cells were cultured as above.

Reagents: PEI-PEG-EGF triconjugate was composed of a PEGylated linear polyethyleneimine conjugated via a MCC linker to hEGF, synthetized according to WO 2015/173824, Example 10. PEI-PEG-EGF triconjugate and pIC were used to prepare PEI-PEG-EGF/polyIC polyplexes in HBG (HEPES-Buffered Glucose). JetPEI (Polyplus) and pIC were used to prepare jetPEI-pIC polyplexes according to manufacturer's instructions. Lipofectamin RNAiMax (Invitrogen, P/N 56531) and pIC were used to prepare RNAiMax-pIC polyplexes according to the manufacturer's instructions. Poly-L-glutamic acid (pLGA; Sigma 50-100 kDa) and PEI-PEG-EGF were used to prepare PEI-PEG-EGF/pLGA polyplexes with the same net charge as the PEI-PEG-EGF/polyIC polyplexes.

Treatment: 3000 cells/well were seeded in 96-well plates. 24 h later, cells were treated with polyplexes at the indicated concentrations (ranging from 2 μg/ml to 0.001 μg/ml). 72 h after treatment, cell survival was evaluated by CellTiter Glo (Promega) and luminescence was recorded using the synergy H1 cell plate reader (Biotek).

Results: The effect of PEI-PEG-EGF/polyIC polyplex on the viability of high and low EGFR-expressing cancer cell lines and non-cancer lines was compared to the effects of naked pIC, jetPEI-pIC, RNAiMax-pIC (lipid based transfection reagent) and PEI-PEG-EGF/pLGA polyplexes.

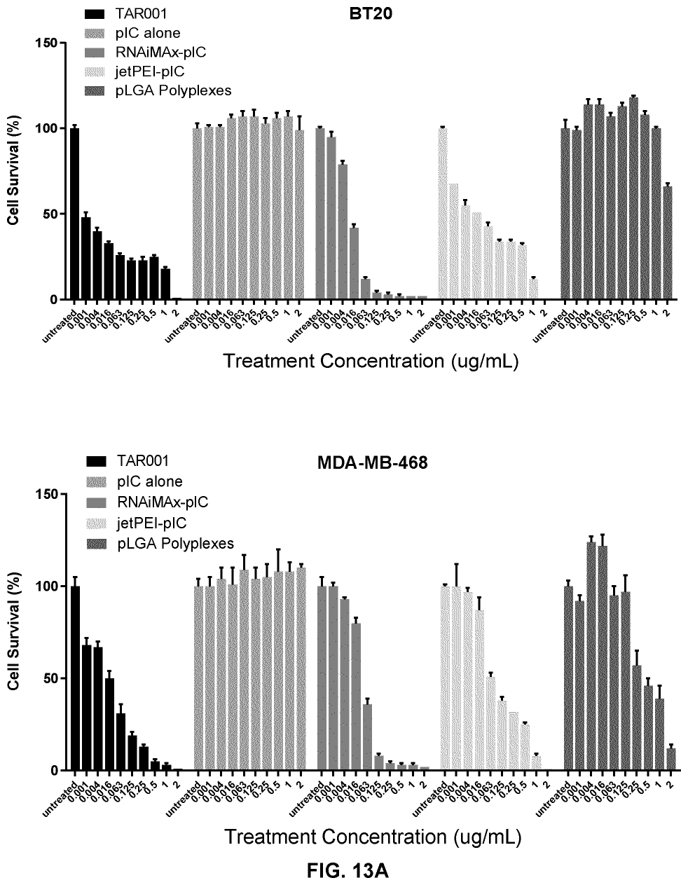

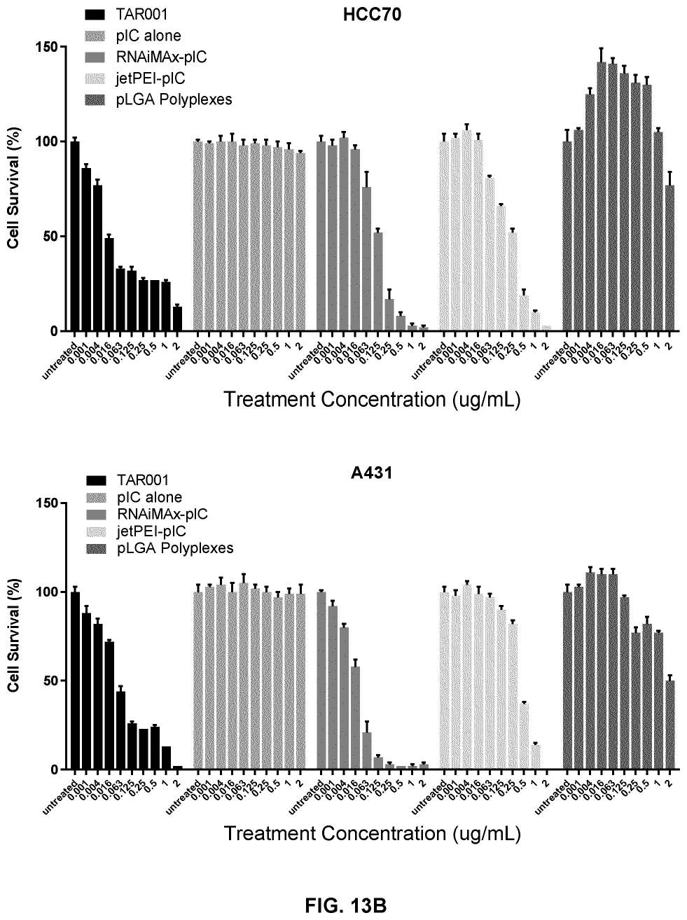

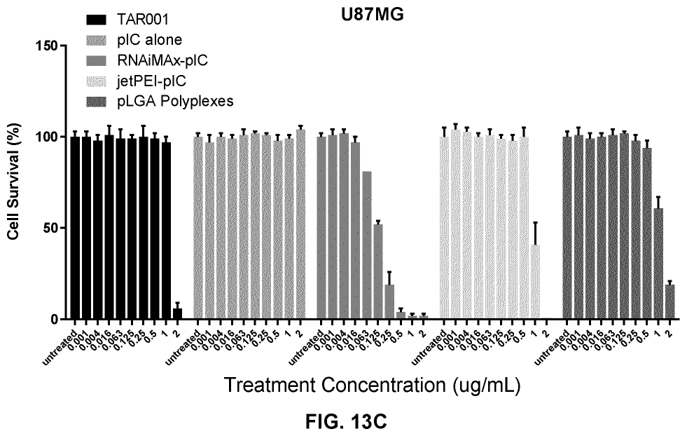

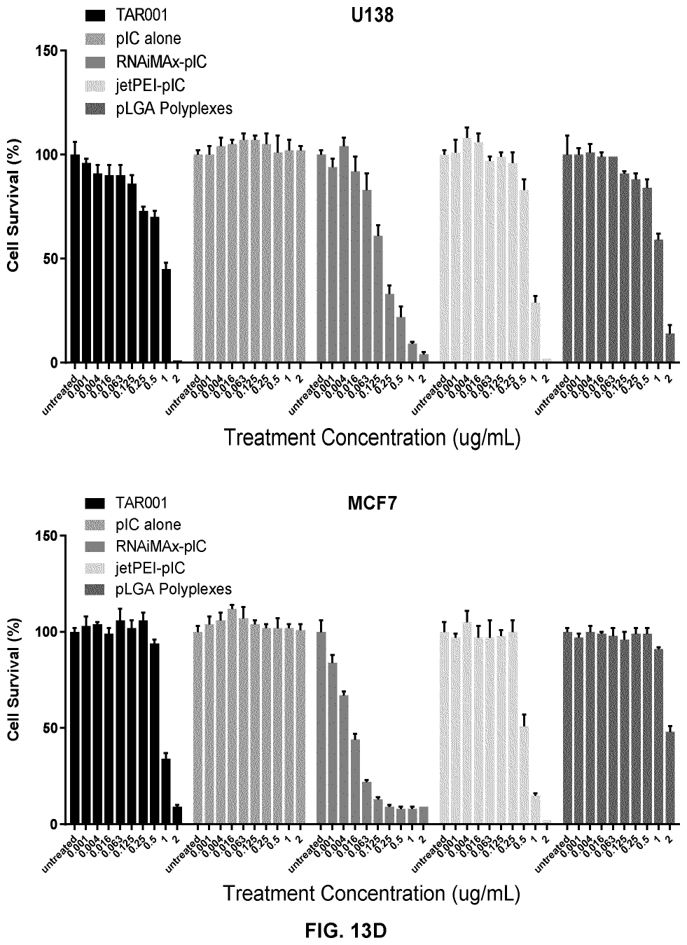

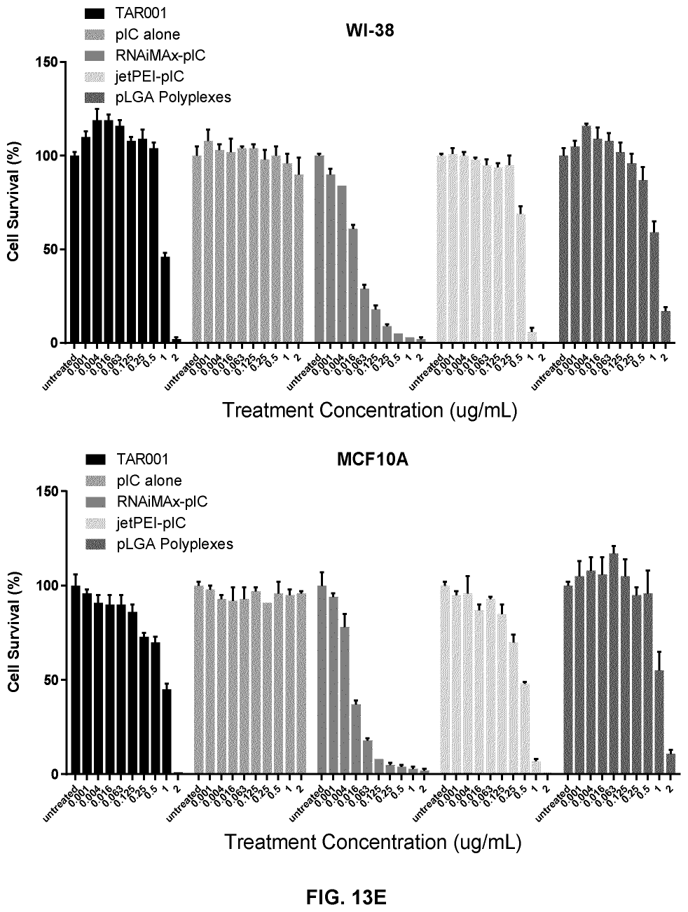

JetPEI-pIC polyplexes were significantly less effective than PEI-PEG-EGF/polyIC polyplex in all EGFR-overexpressing cells: the IC50 of PEI-PEG-EGF/polyIC polyplex was 10-20 fold lower than that of jetPEI-pIC. In cancer cell lines with low EGFR or in non-cancer cell lines jetPEI-pIC was up to 2 fold more toxic than PEI-PEG-EGF/polyIC polyplex (FIG. 13 A-E). These results demonstrate increased efficacy and selectivity of PEI-PEG-EGF/polyIC polyplex as compared to jetPEI-pIC.

Increased efficacy of PEI-PEG-EGF/polyIC polyplex was observed in 3 out of 4 EGFR-overexpressing cell lines as compared to treatment with RNAiMax-pIC. Only in A431 cells was the IC50 of RNAiMax-pIC lower (4-fold) than that of PEI-PEG-EGF/polyIC polyplex (FIG. 13). In BT20, MDA-MB-468 and HCC70 cells, the IC50 of PEI-PEG-EGF/polyIC polyplex was approximately 10-fold lower than that of RNAiMax-pIC. RNAiMax-pIC was found to be toxic in normal cells (WI-3 and MCF10A) as well as in cancer cells with low EGFR expression, while PEI-PEG-EGF/polyIC polyplex was not toxic to these cells.

At the concentrations tested, naked pIC was ineffective in all cell lines.

PEI-PEG-EGF/pLGA polyplexes were significantly less effective (more than 30-fold) than PEI-PEG-EGF/polyIC polyplex against EGFR-overexpressing cancer cells. PEI-PEG-EGF/pLGA polyplexes serve as control to PEI-PEG-EGF/polyIC polyplex. The results clearly demonstrate that the efficacy of PEI-PEG-EGF/polyIC polyplex is mediated via targeted pIC treatment.

PEG-EGF/polyIC polyplex showed high specificity and efficacy in EGFR overexpressing cells, while it did not induce toxicity in normal cell lines. The efficacy of PEI-PEG-EGF/polyIC polyplex was demonstrated to be mediated via pIC. In contrast to PEI-PEG-EGF/polyIC polyplex, RNAiMax/pIC and jetPEI/pIC were less effective and less selective than PEI-PEG-EGF/polyIC polyplex. RNAiMax/pIC was highly toxic in normal cells as well.

1. A kit-of-parts comprising
a. a polyplex comprising a double stranded RNA (dsRNA) and a polymeric conjugate,
wherein said polymeric conjugate comprises a polyethyleneimine (PEI), one or more polyethylene glycol (PEG) moieties and one or more targeting moieties,
wherein said PEI is covalently bound to one or more PEG moieties, and each of said one or more PEG moieties is linked to one of said one or more targeting moieties, and
wherein each of said one or more targeting moieties is capable of binding to a cancer antigen; and
b. at least one antibody, wherein said at least one antibody is capable of modulating an immune checkpoint protein.

2. A composition comprising
a. a polyplex comprising a double stranded RNA (dsRNA) and a polymeric conjugate,
wherein said polymeric conjugate comprises a polyethyleneimine (PEI), one or more polyethylene glycol (PEG) moieties and one or more targeting moieties,
wherein said PEI is covalently bound to one or more PEG moieties, and each of said one or more PEG moieties is linked to one of said one or more targeting moieties, and
wherein each of said one or more targeting moieties is capable of binding to a cancer antigen; and
b. at least one antibody, wherein said at least one antibody is capable of modulating an immune checkpoint protein.

3. The composition of claim 1 or the kit-of-parts of claim 2, wherein said at least one antibody capable of modulating an immune checkpoint protein is
(i) at least one antibody capable of agonizing a co-stimulatory immune checkpoint protein, wherein said co-stimulatory immune checkpoint protein is selected from the group consisting of 4-1BB, 4-1BB ligand (4-1BBL), CD40, CD40 ligand (CD40L), OX40, OX-40 ligand (OX-40L), GITR, GITR ligand (GITRL), ICOS and ICOS ligand (ICOSL); or
(ii) at least one antibody capable of antagonizing a co-inhibitory immune checkpoint protein, wherein said co-inhibitory immune checkpoint protein is selected from the group consisting of PD-1, PD-L1, PD-L2, CTLA-4, B7-H3, B7-H4, VISTA, LAG-3, Galectin-9, TIM-3, and TIGIT; or
(iii) a mixture of at least one antibody of (i) and at least one antibody of (ii).

4. The composition or the kit-of-parts according to any one of the preceding claims, wherein said immune checkpoint protein is selected from the group consisting of 4-1BB, 4-1BB ligand, PD-1, PD-L1, PD-L2 and CTLA-4, more preferably said immune checkpoint protein is selected from the group consisting of 4-1BB, 4-1BB ligand, PD-1, PD-L1, and PD-L2.

5. The composition or the kit-of-parts according to any one of the preceding claims, wherein said immune checkpoint protein is 4-1BB or PD-1.

6. The composition or the kit-of-parts of any one of the preceding claims, wherein said at least one antibody capable of modulating an immune checkpoint protein is a mixture of
(i) an antibody capable of modulating immune checkpoint protein CD27 and an antibody capable of modulating an immune checkpoint protein selected from the group consisting of PD-1, PD-L1, and PD-L2;
(ii) an antibody capable of modulating immune checkpoint protein CD40 and at least one antibody capable of modulating an immune checkpoint protein selected from the group consisting of PD-1, PD-L1, PD-L2, and CTLA-4;
(iii) an antibody capable of modulating immune checkpoint protein GITR and at least one antibody capable of modulating an immune checkpoint protein selected from the group consisting of PD-1, PD-L1, PD-L2, and CTLA-4;
(iv) an antibody capable of modulating immune checkpoint protein OX40 and at least one antibody capable of modulating an immune checkpoint protein selected from the group consisting of PD-1, PD-L1, PD-L2, 4-1BB, and CTLA-4;
(v) an antibody capable of modulating immune checkpoint protein 4-1BB and at least one antibody capable of modulating an immune checkpoint protein selected from the group consisting of PD-1, PD-L1, PD-L2, OX40, LAG-3 and CTLA-4; or
(vi) an antibody capable of modulating immune checkpoint protein ICOS and at least one antibody capable of modulating an immune checkpoint protein selected from the group consisting of PD-1, PD-L1, and PD-L2.

7. The composition or the kit-of-parts according to any one of the preceding claims, wherein said PEI is covalently bound to one, two or three PEG moieties, preferably to one or three PEG moieties.

8. The composition or the kit-of-parts according to any one of the preceding claims, wherein said polyethyleneimine (PEI) is linear polyethyleneimine (LPEI).

9. The composition or the kit-of-parts according to any one of the preceding claims, wherein said dsRNA is polyinosinic-polycytidylic acid double stranded RNA (polyIC).

10. The composition or the kit-of-parts according to any one of the preceding claims, wherein said cancer antigen is EGFR, HER2 or PSMA, preferably said cancer antigen is EGFR.

11. The composition or the kit-of-parts according to any one of the preceding claims, wherein said one or more targeting moieties are selected from the group consisting of EGF, a HER2 affibody, a HER2 antibody, and a DUPA moiety (HOOC(CH₂)₂—CH(COOH)—NH—CO—NH—CH(COOH)—(CH₂)₂—CO—).

12. The composition or the kit-of-parts according to any one of the preceding claims, wherein said one or more targeting moieties is EGF, preferably human EGF.

13. The composition or the kit-of-parts according to any one of the preceding claims for use in the treatment of cancer in a mammal, preferably a human.

14. The composition or the kit-of-parts for use according to claim 13, wherein said cancer is selected from the group consisting of melanoma, non-small-cell lung cancer, breast cancer, ovarian cancer, cervical cancer, endometrial cancer, vulva cancer, urothelial cancer, bladder cancer, renal cancer, esophageal cancer, gastric cancer, pancreatic cancer, colorectal cancer, glial tumor, head and neck cancer, prostate cancer, penile cancer, testicular-embryonal cancer, neuroendocrine tumor, and hepatocellular cancer.

15. The kit-of-parts for use according to claim 13 or claim 14, wherein the polyplex is administered separately from the at least one antibody capable of modulating an immune checkpoint protein to said mammal, preferably human.
